(19) United States
(12) Patent Application Publication
(10) Pub. No.: US 2026/0234177 A1
(43) Pub. Date: Aug. 13, 2026

(54) PURIFICATION OF A SACCHARIDE FROM A FERMENTATION BROTH

(71) Applicant: Inbiose N.V., Zwijnaarde (BE)

(72) Inventors: Jordy Bauwelinck, Zwijnaarde (BE); Gert Peters, Zwijnaarde (BE); Dries Van Herpe, Zwijnaarde (BE); Ward Vannevel, Zwijnaarde (BE)

(21) Appl. No.: 19/149,329

(22) PCT Filed: Jan. 19, 2024
(86) PCT No.: PCT/EP2024/051240

§ 371 Date: Jul. 18, 2025

(30) Foreign Application Priority Data
Jan. 19, 2023 (EP) 23152418.2
Jan. 19, 2023 (LU) LU503381

(51) Int. Cl. C07H 1/06; B01D 61/02; B01D 61/04; B01D 61/16; B01D 61/58; B01D 69/02; B01D 71/56

(52) U.S. Cl. CPC C07H 1/06; B01D 61/58; B01D 69/02; B01D 71/56; B01D 61/027; B01D 61/04; B01D 61/16; B01D 2315/16; B01D 2325/02832; B01D 2325/20

(57) ABSTRACT

The present invention relates to methods for the purification of a saccharide from a fermentation broth, a product of such processes, and the use of a product of such processes.

### FIELD OF THE INVENTION

The present invention relates to methods for the purification of a saccharide from a fermentation broth, a product of such processes, and the use of a product of such processes.

### BACKGROUND

Saccharides like e.g., disaccharides and oligosaccharides, are very diverse in chemical structure and are composed of miscellaneous monosaccharides, such as e.g., glucose, galactose, N-acetylglucosamine, xylose, rhamnose, fucose, mannose, N-acetylneuraminic acid, N-acetylgalactosamine, galactosamine, glucosamine, glucuronic acid, galacturonic acid. Saccharides are widely distributed in all living organisms and play important roles in a variety of physiological and pathological processes, such as cell metastasis, signal transduction, intercellular adhesion, inflammation, and immune response. Economical production of these saccharides is of utmost importance to fully benefit of their biological advantages. An important group of saccharides comprises mammalian milk oligosaccharides (MMOs) and human milk oligosaccharides (HMOs) found in mammalian and human milk, respectively. A wide variety of synthesis methods have been developed already, ranging from extraction over chemical synthesis to enzymatic synthesis. These methods are currently least applied, whereas biotechnological fermentative production is nowadays pursued and commercialized. Methods for the production of saccharides, in particular oligosaccharides, are reviewed by Lu et al (2021), Faijes et al (2019), Kruschitz et al (2020), Ghosh et al (2020), Vera et al (2021), Walsh et al (2020), Li et al (2020), Li and Ye (2020) and are well known for a person skilled in the art. After production, the saccharide needs to be purified.

### DESCRIPTION

### Summary of the Invention

It is an object of the present invention to provide for methods by means of which a saccharide can be purified from a fermentation broth, preferably in an efficient, time and cost-effective way and which yields a high quality, high purity and good yield of the desired saccharide.

According to the invention, this and other objects are achieved by providing a method for the purification of a saccharide from a fermentation broth comprising said saccharide and biomass. The method comprises filtration of said fermentation broth on a membrane and subsequent diafiltration on said membrane under conditions permissive to produce a retentate comprising said biomass and a permeate comprising said saccharide, wherein the membrane used in said filtration and diafiltration comprises a molecular weight cut-off ranging from 0.3 to 5 kDa, a pore size ranging from 0.001 to 0.01 μm, and/or a monovalent ion rejection ranging from 1 to 50%. This invention also provides a purified saccharide by the above-referenced method. Furthermore, this invention provides a purified saccharide mixture comprising a purified saccharide by the above-referenced method. Further benefits of the teachings of this invention will be apparent to one skilled in the art from reading this invention.

### Definitions

The words used in this specification to describe the invention and its various embodiments are to be understood not only in the sense of their commonly defined meanings, but to include by special definition in this specification structure, material or acts beyond the scope of the commonly defined meanings. Thus, if an element can be understood in the context of this specification as including more than one meaning, then its use in a claim must be understood as being generic to all possible meanings supported by the specification and by the word itself.

The various aspects and embodiments of the invention disclosed herein are to be understood not only in the order and context specifically described in this specification, but to include any order and any combination thereof. Each embodiment as identified herein may be combined together unless otherwise indicated. All publications, patents, and patent applications mentioned in this specification are herein incorporated by reference to the same extent as if each individual publication, patent, or patent application was specifically and individually indicated to be incorporated by reference in its entirety.

Unless specifically stated otherwise, all words used in the singular number shall be deemed to include the plural and vice versa. Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry and nucleic acid chemistry and hybridization described herein are those well-known and commonly employed in the art. Standard techniques are used for nucleic acid and peptide synthesis. Generally, enzymatic reactions and purification steps are performed according to the manufacturer's specifications.

In the specification, there have been disclosed embodiments of the invention, and although specific terms are employed, the terms are used in a descriptive sense only and not for purposes of limitation, the scope of the invention being set forth in the following claims. It must be understood that the illustrated embodiments have been set forth only for the purposes of example and that it should not be taken as limiting the invention. It will be apparent to those skilled in the art that alterations, other embodiments, improvements, details and uses can be made consistent with the letter and spirit of the disclosure herein and within the scope of this disclosure, which is limited only by the claims, construed in accordance with the patent law, including the doctrine of equivalents. In the claims that follow, reference characters used to designate claim steps are provided for convenience of description only, and are not intended to imply any particular order for performing the steps, unless specifically stated otherwise.

Throughout the application, unless explicitly stated otherwise, the features “synthesize”, “synthesized” and “synthesis” are interchangeably used with the features “produce”, “produced” and “production”, respectively. Throughout the application, unless explicitly stated otherwise, the expressions “capable of . . . <verb>” and “capable to . . . <verb>” are preferably replaced with the active voice of said verb and vice versa. For example, the expression “capable of expressing” is preferably replaced with “expresses” and vice versa, i.e., “expresses” is preferably replaced with “capable of expressing”. Throughout this document and in its claims, the verbs “to comprise”, “to have” and “to contain” and their conjugations are used in their non-limiting sense to mean that items following the word are included, but items not specifically mentioned are not excluded. The verb “to consist essentially of” means that a solution or a composition as defined herein may comprise additional component(s) than the ones specifically identified, said additional component(s) not altering the unique characteristic of the invention. Said additional compound(s) might be inevitable by-product(s), for example, generated during production of the saccharide or the saccharide mixture of present invention as well as compound(s) that were introduced into a process stream from which the saccharide or the saccharide mixture is recovered but which could not have been removed therefrom. The term “consisting essentially of” with respect to spray-dried powders includes spray-dried powders containing with respect to the dry matter of the spray-dried powder at least 80%-wt., at least 85%-wt., at least 90%-wt., at least 93%-wt., at least 95%-wt. or at least 98%-wt. of the saccharide or the saccharide mixture. The term “consisting essentially of” is used likewise with respect to spray-dried powders, process streams and solutions containing the saccharide or the saccharide mixture. Throughout this document and in its claims, unless specifically stated otherwise, the verbs “to comprise”, “to have” and “to contain”, and their conjugations, may be preferably replaced by “to consist of” (and its conjugations) or “to consist essentially of” (and its conjugations) and vice versa. In addition, reference to an element by the indefinite article “a” or “an” does not exclude the possibility that more than one of the elements is present, unless the context clearly requires that there be one and only one of the elements. The indefinite article “a” or “an” thus usually means “at least one”. Throughout the document and in the claims, unless explicitly stated otherwise, the articles “a” and “an” are preferably replaced by “at least one”, more preferably “at least two”, even more preferably by “at least three”, even more preferably by “at least four”, even more preferably by “at least five”, even more preferably by “at least six”, most preferably by “at least two”. The word “about” or “approximately” when used in association with a numerical value (e.g., “about 10”) or with a range (e.g., “about x to approximately y”) preferably means that the value or range is interpreted as being as accurate as the method used to measure it. If no error margins are specified, the expression “about” or “approximately” when used in association with a numerical value is interpreted as having the same round-off as the given value. Throughout this document and its claims, unless otherwise stated, the expression “from x to y”, wherein x and y represent numerical values, refers to a range of numerical values wherein x is the lower value of the range and y is the upper value of the range. Herein, x and y are also included in the range.

The term “saccharide” as used herein refers to a sugar chosen from the list comprising monosaccharide, disaccharide, oligosaccharide and polysaccharide.

The term “monosaccharide” as used herein refers to a sugar that is not decomposable into simpler sugars by hydrolysis, is classed as an aldose, a ketose, a deoxysugar, a deoxy-aminosugar, a uronic acid, an aldonic acid, a ketoaldonic acid, an aldaric acid or a sugar alcohol, and contains one or more hydroxyl groups per molecule. Monosaccharides are saccharides containing only one simple sugar.

The term “phosphorylated monosaccharide” as used herein refers to a monosaccharide which is phosphorylated. Examples of phosphorylated monosaccharides include but are not limited to glucose-1-phosphate, glucose-6-phosphate, glucose-1,6-bisphosphate, galactose-1-phosphate, fructose-6-phosphate, fructose-1,6-bisphosphate, fructose-1-phosphate, glucosamine-1-phosphate, glucosamine-6-phosphate, N-acetylglucosamine-1-phosphate, mannose-1-phosphate, mannose-6-phosphate or fucose-1-phosphate.

The terms “activated monosaccharide”, “nucleotide-activated sugar”, “nucleotide-sugar”, “activated sugar”, “nucleoside” or “nucleotide donor” are used herein interchangeably and refer to activated forms of monosaccharides. Examples of activated monosaccharides include but are not limited to UDP-N-acetylglucosamine (UDP-GlcNAc), UDP-N-acetylgalactosamine (UDP-GalNAc), UDP-N-acetylmannosamine (UDP-ManNAc), UDP-glucose (UDP-Glc), UDP-galactose (UDP-Gal), GDP-mannose (GDP-Man), UDP-glucuronate, UDP-galacturonate, UDP-2-acetamido-2,6-dideoxy-L-arabino-4-hexulose, UDP-2-acetamido-2,6-dideoxy-L-lyxo-4-hexulose, UDP-N-acetyl-L-rhamnosamine (UDP-L-RhaNAc or UDP-2-acetamido-2,6-dideoxy-L-mannose), dTDP-N-acetylfucosamine, UDP-N-acetylfucosamine (UDP-L-FucNAc or UDP-2-acetamido-2,6-dideoxy-L-galactose), UDP-N-acetyl-L-pneumosamine (UDP-L-PneNAC or UDP-2-acetamido-2,6-dideoxy-L-talose), UDP-N-acetylmuramic acid, UDP-N-acetyl-L-quinovosamine (UDP-L-QuiNAc or UDP-2-acetamido-2,6-dideoxy-L-glucose), GDP-L-quinovose, CMP-sialic acid (CMP-Neu5Ac or CMP-N-acetylneuraminic acid), GDP-fucose (GDP-Fuc), GDP-rhamnose and UDP-xylose. Nucleotide-sugars act as glycosyl donors in glycosylation reactions. Glycosylation reactions are reactions that are catalysed by glycosyltransferases.

The term “glycosyltransferase” as used herein refers to an enzyme capable to catalyse the transfer of a sugar moiety of a donor to a specific acceptor, forming glycosidic bonds. Said donor can be a precursor as defined herein. A classification of glycosyltransferases using nucleotide diphospho-sugar, nucleotide monophospho-sugar and sugar phosphates and related proteins into distinct sequence-based families has been described (Campbell et al., Biochem. J. 326, 929-939 (1997)) and is available on the CAZy (CArbohydrate-Active EnZymes) website (www.cazy.org). As used herein the glycosyltransferase can be selected from the list comprising but not limited to: fucosyltransferases, sialyltransferases, galactosyltransferases, glucosyltransferases, mannosyltransferases, N-acetylglucosaminyltransferases, N-acetylgalactosaminyltransferases, N-acetylmannosaminyltransferases, xylosyltransferases, glucuronyltransferases, galacturonyltransferases, glucosaminyltransferases, N-glycolylneuraminyltransferases, rhamnosyltransferases, N-acetylrhamnosyltransferases, UDP-4-amino-4,6-dideoxy-N-acetyl-beta-L-altrosamine transaminases, UDP-N-acetylglucosamine enolpyruvyl transferases and fucosaminyltransferases.

The term “disaccharide” as used herein refers to a saccharide polymer containing two simple sugars, i.e., monosaccharides. Examples of disaccharides comprise lactose (Gal-β1,4-Glc), lacto-N-biose (Gal-β1,3-GlcNAc), N-acetyllactosamine (Gal-β1,4-GlcNAc), LacDiNAc (GalNAc-β1,4-GlcNAc), N-acetylgalactosaminylglucose (GalNAc-β1,4-Glc), Neu5Ac-α2,3-Gal, Neu5Ac-α2,6-Gal, fucopyranosyl-(1-4)-N-glycolylneuraminic acid (Fuc-(1-4)-Neu5Gc), sucrose (Glc-α1,2-Fru), maltose (Glc-α1,4-Glc) and melibiose (Gal-α1,6-Glc).

“Oligosaccharide” as the term is used herein and as generally understood in the state of the art, refers to a saccharide polymer containing a small number, typically three to twenty, preferably three to ten, of simple sugars, i.e., monosaccharides. The oligosaccharide as used in the present invention can be a linear structure or can include branches. The linkage (e.g., glycosidic linkage, galactosidic linkage, glucosidic linkage, etc.) between two sugar units can be expressed, for example, as 1,4, 1->4, or (1-4), used interchangeably herein. For example, the terms “Gal-b1,4-Glc”, “Gal-β1,4-Glc”, “b-Gal-(1->4)-Glc”, “β-Gal-(1->4)-Glc”, “Galbeta1-4-Glc”, “Gal-b(1-4)-Glc” and “Gal-β(1-4)-Glc” have the same meaning, i.e. a beta-glycosidic bond links carbon-1 of galactose (Gal) with the carbon-4 of glucose (Glc). Each monosaccharide can be in the cyclic form (e.g., pyranose or furanose form). Linkages between the individual monosaccharide units may include alpha 1->2, alpha 1->3, alpha 1->4, alpha 1->6, alpha 2->1, alpha 2->3, alpha 2->4, alpha 2->6, beta 1->2, beta 1->3, beta 1->4, beta 1->6, beta 2->1, beta 2->3, beta 2->4, and beta 2->6. An oligosaccharide can contain both alpha- and beta-glycosidic bonds or can contain only alpha-glycosidic or only beta-glycosidic bonds. The term “polysaccharide” refers to a compound consisting of a large number, typically more than twenty, of monosaccharides linked glycosidically. Examples of oligosaccharides include but are not limited to Lewis-type antigen oligosaccharides, mammalian (including human) milk oligosaccharides, 0-antigen, enterobacterial common antigen (ECA), the glycan chain present in lipopolysaccharides (LPS), the oligosaccharide repeats present in capsular polysaccharides, peptidoglycan (PG), amino-sugars, antigens of the human ABO blood group system, animal oligosaccharides, preferably selected from the list consisting of N-glycans and O-glycans, plant oligosaccharides, preferably selected from the list consisting of N-glycans and O-glycans, sialylated oligosaccharides, neutral (non-charged) oligosaccharides, negatively charged oligosaccharides, fucosylated oligosaccharides, N-acetylglucosamine containing oligosaccharides, lacto-N-biose containing oligosaccharides, N-acetyllactosamine-containing oligosaccharides, N-acetylglucosamine containing sialylated oligosaccharides, N-acetylglucosamine containing neutral (non-charged) oligosaccharides, N-acetylglucosamine containing negatively charged oligosaccharides, N-acetylglucosamine containing fucosylated oligosaccharides, N-acetylglucosamine containing non-fucosylated oligosaccharides, lacto-N-biose containing sialylated oligosaccharides, lacto-N-biose containing neutral (non-charged) oligosaccharides, lacto-N-biose containing negatively charged oligosaccharides, lacto-N-biose containing fucosylated oligosaccharides, lacto-N-biose containing non-fucosylated oligosaccharides, N-acetyllactosamine containing sialylated oligosaccharides, N-acetyllactosamine containing neutral (non-charged) oligosaccharides, N-acetyllactosamine containing negatively charged oligosaccharides, N-acetyllactosamine containing fucosylated oligosaccharides, N-acetyllactosamine containing non-fucosylated oligosaccharides, chitosan, chitosan comprising oligosaccharide, heparosan, chondroitin sulphate, glycosaminoglycan oligosaccharide, heparin, heparan sulphate, dermatan sulphate, hyaluronan, hyaluronic acid and keratan sulphate.

The terms “negatively charged oligosaccharide” or “acidic oligosaccharide” are used interchangeably and refer to an oligosaccharide with a negative charge. In a preferred embodiment, the negatively charged oligosaccharide is a sialylated oligosaccharide. As used herein, a ‘sialylated oligosaccharide’ is to be understood as a negatively charged sialic acid containing oligosaccharide, i.e., an oligosaccharide having one or more sialic acid residue(s). It has an acidic nature. Some examples are 3′SL (3′-sialyllactose, Neu5Ac-α2,3-Gal-β1,4-Glc), 3′-sialyllactosamine, 6′SL (6′sialyllactose, Neu5Ac-α2,6-Gal-β1,4-Glc), 8′SL (8′sialyllactose, Neu5Ac-α2,8-Gal-β1,4-Glc), 3,6-disialyllactose (Neu5Ac-α2,3-(Neu5Ac-α2,6)-Gal-β1,4-Glc), 6,6′-disialyllactose (Neu5Ac-α2,6-Gal-β1,4-(Neu5Ac-α2,6)-Glc), 8,3-disialyllactose (Neu5Ac-α2,8-Neu5Ac-α2,3-Gal-β1,4-Glc), 6′-sialyllactosamine, oligosaccharides comprising 6′sialyllactose, SGG hexasaccharide (Neu5Aca-2,3Galβ-1,3GalNacβ-1,3Gala-1,4Galp-1,4Gal), sialylated tetrasaccharide, sialylated pentasaccharide, sialylated lacto-N-triose, sialylated lacto-N-tetraose, sialyllacto-N-neotetraose, LSTc (Neu5Ac-α2,6-Gal-β1,4-GlcNAc-β1,3-Gal-β1,4-Glc), LSTd (Neu5Ac-α2,3-Gal-β1,4-GlcNAc-β1,3-Gal-β1,4-Glc), monosialyllacto-N-hexaose, disialyllacto-N-hexaose I, monosialyllacto-N-neohexaose I, monosialyllacto-N-neohexaose II, disialyllacto-N-neohexaose, disialyllacto-N-tetraose, disialyllacto-N-hexaose II, sialyllacto-N-tetraose a (LSTa, Neu5Ac-α2,3-Gal-β1,3-GlcNAc-β1,3-Gal-β1,4-Glc), disialyllacto-N-hexaose I, sialyllacto-N-tetraose b (LSTb, Gal-β1,3-(Neu5Ac-α2,6)-GlcNAc-β1,3-Gal-(β1,4-Glc), 3′-sialyl-3-fucosyllactose, fucodisialyllacto-N-hexaose, disialomonofucosyllacto-N-neohexaose, monofucosylmonosialyllacto-N-octaose (sialyl Lea), sialyllacto-N-fucohexaose II, disialyllacto-N-fucopentaose II, monofucosyldisialyllacto-N-tetraose and oligosaccharides bearing one or several sialic acid residue(s).

“Charged oligosaccharides” are oligosaccharide structures that contain one or more negatively charged monosaccharide subunits including N-acetylneuraminic acid (Neu5Ac), commonly known as sialic acid, N-glycolylneuraminic acid (Neu5Gc), glucuronate and galacturonate. Charged oligosaccharides are also referred to as acidic oligosaccharides. Sialic acid belongs to the family of derivatives of neuraminic acid (5-amino-3,5-dideoxy-D-glycero-D-galacto-non-2-ulosonic acid). Neu5Gc is a derivative of sialic acid, which is formed by hydroxylation of the N-acetyl group at C5 of Neu5Ac. In contrast, neutral (non-charged) oligosaccharides are non-sialylated oligosaccharides, and thus do not contain an acidic monosaccharide subunit. Neutral oligosaccharides comprise non-charged fucosylated oligosaccharides that contain one or more fucose subunits in their glycan structure as well as non-charged non-fucosylated oligosaccharides that lack any fucose subunit. Other examples of charged oligosaccharides are sulphated chitosans and deacetylated chitosans.

The terms ‘neutral oligosaccharide’ and ‘non-charged’ oligosaccharide as used herein are used interchangeably and refer, as generally understood in the state of the art, to an oligosaccharide that has no negative charge originating from a carboxylic acid group. Examples of such neutral oligosaccharide are 2′-fucosyllactose (2′FL), 3-fucosyllactose (3FL), 4-fucosyllactose (4FL), 6-fucosyllactose (6FL), 2′,3-difucosyllactose (diFL), lacto-N-triose II (LN3), lacto-N-tetraose (LNT), lacto-N-neotetraose (LNnT), lacto-N-fucopentaose 1, lacto-N-neofucopentaose 1, lacto-N-fucopentaose 11, lacto-N-fucopentaose III, lacto-N-fucopentaose V, lacto-N-fucopentaose VI, lacto-N-neofucopentaose V, lacto-N-difucohexaose 1, lacto-N-difucohexaose II, 6′-galactosyllactose, 3′-galactosyllactose, lacto-N-hexaose, lacto-N-neohexaose, para-lacto-N-hexaose, para-lacto-N-neohexaose, difucosyl-lacto-N-hexaose and difucosyl-lacto-N-neohexaose.

A ‘fucosylated oligosaccharide’ as used herein and as generally understood in the state of the art is an oligosaccharide that is carrying a fucose-residue. Such fucosylated oligosaccharide is a saccharide structure comprising at least three monosaccharide subunits linked to each other via glycosidic bonds, wherein at least one of said monosaccharide subunit is a fucose. A fucosylated oligosaccharide can contain more than one fucose residue, e.g., two, three or more. A fucosylated oligosaccharide can be a neutral oligosaccharide or a charged oligosaccharide e.g., also comprising sialic acid structures. Fucose can be linked to other monosaccharide subunits comprising glucose, galactose, GlcNAc via alpha-glycosidic bonds comprising alpha-1,2 alpha-1,3, alpha-1,4, alpha-1,6 linkages. Examples comprise 2′-fucosyllactose (2′FL), 3-fucosyllactose (3FL), 4-fucosyllactose (4FL), 6-fucosyllactose (6FL), difucosyllactose (diFL), Lacto-N-fucopentaose I (LNFP 1), Lacto-N-fucopentaose II (LNFP 11), Lacto-N-fucopentaose 11 (LNFP III), lacto-N-fucopentaose V (LNFP V), lacto-N-fucopentaose VI (LNFP VI), lacto-N-neofucopentaose 1, lacto-N-difucohexaose I (LDFH 1), lacto-N-difucohexaose II (LDFH 11), Monofucosyllacto-N-hexaose 11 (MFLNH 111), Difucosyllacto-N-hexaose (DFLNHa), difucosyl-lacto-N-neohexaose, 3′-sialyl-3-fucosyllactose, disialomonofucosyllacto-N-neohexaose, monofucosylmonosialyllacto-N-octaose (sialyl Lea), sialyllacto-N-fucohexaose 11, disialyllacto-N-fucopentaose 11, monofucosyldisialyllacto-N-tetraose.

Mammalian milk oligosaccharides or MMOs comprise oligosaccharides present in milk found in any phase during lactation including colostrum milk from humans (i.e. human milk oligosaccharides or HMOs) and mammals including but not limited to cows (Bos taurus), sheep (Ovis aries), goats (Capra aegagrus hircus), bactrian camels (Camelus bactrianus), horses (Equusferus caballus), pigs (Sus scropha), dogs (Canis lupus familiaris), ezo brown bears (Ursus arctos yesoensis), polar bear (Ursus maritimus), Japanese black bears (Ursus thibetanus japonicus), striped skunks (Mephitis mephitis), hooded seals (Cystophora cristata), Asian elephants (Elephas maximus), African elephant (Loxodonta africana), giant anteater (Myrmecophaga tridactyla), common bottlenose dolphins (Tursiops truncates), northern minke whales (Balaenoptera acutorostrata), tammar wallabies (Macropus eugenii), red kangaroos (Macropus rufus), common brushtail possum (Trichosurus vulpecula), koalas (Phascolarctos cinereus), eastern quolls (Dasyurus viverrinus), platypus (Ornithorhynchus anatinus). As used herein, “mammalian milk oligosaccharide” or “MMO” refers to oligosaccharides such as but not limited to 3-fucosyllactose, 2′-fucosyllactose, 6-fucosyllactose, 2′,3-difucosyllactose, 2′,2-difucosyllactose, 3,4-difucosyllactose, 6′-sialyllactose, 3′-sialyllactose, 3,6-disialyllactose, 6,6′-disialyllactose, 8,3-disialyllactose, 3,6-disialyllacto-N-tetraose, lacto-N-tetraose, lacto-N-neotetraose, lacto-N-fucopentaose II, lacto-N-fucopentaose I, lacto-N-fucopentaose III, lacto-N-fucopentaose V, lacto-N-fucopentaose VI, sialyllacto-N-tetraose c, sialyllacto-N-tetraose b, sialyllacto-N-tetraose a, lacto-N-difucohexaose I, lacto-N-difucohexaose II, lacto-N-hexaose, lacto-N-neohexaose, para-lacto-N-hexaose, monofucosylmonosialyllacto-N-tetraose c, monofucosyl para-lacto-N-hexaose, monofucosyllacto-N-hexaose III, isomeric fucosylated lacto-N-hexaose III, isomeric fucosylated lacto-N-hexaose I, sialyllacto-N-hexaose, sialyllacto-N-neohexaose II, difucosyl-para-lacto-N-hexaose, difucosyllacto-N-hexaose, difucosyllacto-N-hexaose a, difucosyllacto-N-hexaose c, galactosylated chitosan, fucosylated oligosaccharides, neutral oligosaccharides and/or sialylated oligosaccharides.

The terms “human milk oligosaccharide” or “HMO” refer to oligosaccharides found in human breast milk, including preterm human milk, colostrum and term human milk. HMOs comprise fucosylated oligosaccharides, non-fucosylated neutral oligosaccharides and sialylated oligosaccharides (see e.g., Chen X., Chapter Four: Human Milk Oligosaccharides (HMOS): Structure, Function, and Enzyme-Catalyzed Synthesis in Adv. Carbohydr. Chem. Biochem. 72, 113 (2015)). Examples of HMOs comprise 3-fucosyllactose, 2′-fucosyllactose, 2′,3-difucosyllactose, 6′-sialyllactose, 3′-sialyllactose, LN3, lacto-N-tetraose, lacto-N-neotetraose, lacto-N-fucopentaose II, lacto-N-fucopentaose I, lacto-N-fucopentaose III, lacto-N-fucopentaose V, lacto-N-fucopentaose VI, sialyllacto-N-tetraose c, sialyllacto-N-tetraose b, sialyllacto-N-tetraose a, difucosyllacto-N-tetraose, lacto-N-hexaose, lacto-N-difucohexaose I, lacto-N-difucohexaose II, disialyllacto-N-tetraose, fucosyllacto-N-hexaose, difucosyllacto-N-hexaose, fucodisialyllacto-N-hexaose, disialyllacto-N-hexaose.

The terms “sialic acid”, “N-acetylneuraminate”, “N-acylneuraminate”, “N-acetylneuraminic acid” and “Neu(n)Ac molecule” are used interchangeably and refer to an acidic sugar with a nine-carbon backbone comprising but not limited to Neu4Ac; Neu5Ac; Neu4,5Ac2; Neu5,7Ac2; Neu5,8Ac2; Neu5,9Ac2; Neu4,5,9Ac3; Neu5,7,9Ac3; Neu5,8,9Ac3; Neu4,5,7,9Ac4; Neu5,7,8,9Ac4 and Neu4,5,7,8,9Ac5 and Neu5Gc.

“Recombinant” means genetically engineered DNA prepared by transplanting or splicing genes from one species into the cells of a host organism of a different species. Such DNA becomes part of the host's genetic makeup and is replicated. The terms “recombinant” or “transgenic” or “metabolically engineered” or “genetically engineered” as used herein with reference to a cell or host cell are used interchangeably and indicates that the cell replicates a heterologous nucleic acid, or expresses a peptide or protein encoded by a heterologous nucleic acid (i.e., a sequence “foreign to said cell” or a sequence “foreign to said location or environment in said cell”). Such cells are described to be transformed with at least one heterologous or exogenous gene or are described to be transformed by the introduction of at least one heterologous or exogenous gene. Recombinant or metabolically engineered cells can contain genes that are not found within the native (non-recombinant) form of the cell. Recombinant cells can also contain genes found in the native form of the cell wherein the genes are modified and re-introduced into the cell by artificial means. The terms also encompass cells that contain a nucleic acid endogenous to the cell that has been modified or its expression or activity has been modified without removing the nucleic acid from the cell; such modifications include those obtained by gene replacement, replacement of a promoter; site-specific mutation; and related techniques. Accordingly, a “recombinant polypeptide” is one which has been produced by a recombinant cell. The terms also encompass cells that have been modified by removing a nucleic acid endogenous to the cell by means of common well-known technologies for a skilled person (like e.g., knocking-out genes).

Protein or polypeptide sequence information and functional information can be provided by a comprehensive resource for protein sequence and annotation data like e.g., the Universal Protein Resource (UniProt) (www.uniprot.org) (Nucleic Acids Res. 2021, 49(D1), D480-D489). UniProt comprises the expertly and richly curated protein database called the UniProt Knowledgebase (UniProtKB), together with the UniProt Reference Clusters (UniRef) and the UniProt Archive (UniParc). The UniProt identifiers (UniProt ID) are unique for each protein present in the database. Throughout the application, the sequence of a polypeptide is represented by an UniProt ID. Unless stated otherwise, the UniProt IDs of the proteins described correspond to their sequence version 01 as present in the UniProt Database (www.uniprot.org) version release 2021_03 and consulted on 9 Jun. 2021. It should be understood for those skilled in the art that for the databases used herein, comprising UniProt, the content of each database is fixed at each release and is not to be changed. When the content of a specific database is changed, this specific database receives a new release version with a new release date. All release versions for each database with their corresponding release dates and specific content as annotated at these specific release dates are available and known to those skilled in the art.

As used herein, the term “mammary cell(s)” generally refers to mammalian mammary epithelial cell(s), mammalian mammary-epithelial luminal cell(s), or mammalian epithelial alveolar cell(s), or any combination thereof. As used herein, the term “mammary-like cell(s)” generally refers to mammalian cell(s) having a phenotype/genotype similar (or substantially similar) to natural mammalian mammary cell(s) but is/are derived from mammalian non-mammary cell source(s). Such mammalian mammary-like cell(s) may be engineered to remove at least one undesired genetic component and/or to include at least one predetermined genetic construct that is typical of a mammalian mammary cell. Non-limiting examples of mammalian mammary-like cell(s) may include mammalian mammary epithelial-like cell(s), mammalian mammary epithelial luminal-like cell(s), mammalian non-mammary cell(s) that exhibits one or more characteristics of a cell of a mammalian mammary cell lineage, or any combination thereof. Further non-limiting examples of mammalian mammary-like cell(s) may include mammalian cell(s) having a phenotype similar (or substantially similar) to natural mammalian mammary cell(s), or more particularly a phenotype similar (or substantially similar) to natural mammalian mammary epithelial cell(s). A mammalian cell with a phenotype or that exhibits at least one characteristic similar to (or substantially similar to) a natural mammalian mammary cell or a mammalian mammary epithelial cell may comprise a mammalian cell (e.g., derived from a mammary cell lineage or a non-mammary cell lineage) that exhibits either naturally, or has been engineered to, be capable of expressing at least one milk component. As used herein, the term “non-mammary cell(s)” may generally include any mammalian cell of non-mammary lineage. In the context of the invention, a non-mammary cell can be any mammalian cell capable of being engineered to express at least one milk component. Non-limiting examples of such non-mammary cell(s) include hepatocyte(s), blood cell(s), kidney cell(s), cord blood cell(s), epithelial cell(s), epidermal cell(s), myocyte(s), fibroblast(s), mesenchymal cell(s), or any combination thereof. In some instances, molecular biology and genome editing techniques can be engineered to eliminate, silence, or attenuate myriad genes simultaneously.

The term “precursor” as used herein refers to substances which are taken up or synthetized by the cell for the specific production of a saccharide according to the present invention. In this sense a precursor can be an acceptor as defined herein, but can also be another substance, metabolite, which is first modified within the cell as part of the biochemical synthesis route of a saccharide. The term “precursor” as used herein is also to be understood as a donor that is used by a glycosyltransferase to modify an acceptor as defined herein with a sugar moiety in a glycosidic bond, as part in the metabolic pathway of a saccharide. Examples of such precursors comprise the acceptors as defined herein, and/or dihydroxyacetone, glucosamine, N-acetylglucosamine, N-acetylmannosamine, galactosamine, N-acetylgalactosamine, galactosyllactose, phosphorylated sugars or sugar phosphates like e.g. but not limited to glucose-1-phosphate, galactose-1-phosphate, glucose-6-phosphate, fructose-6-phosphate, fructose-1,6-bisphosphate, mannose-6-phosphate, mannose-1-phosphate, glycerol-3-phosphate, glyceraldehyde-3-phosphate, dihydroxyacetone-phosphate, glucosamine-6-phosphate, N-acetylglucosamine-6-phosphate, N-acetylmannosamine-6-phosphate, N-acetylglucosamine-1-phosphate, N-acetylneuraminic acid-9-phosphate and nucleotide-activated sugars like nucleotide diphospho-sugars and nucleotide monophospho-sugars as defined herein like e.g. UDP-glucose, UDP-galactose, UDP-N-acetylglucosamine, CMP-sialic acid, GDP-mannose, GDP-4-dehydro-6-deoxy-α-D-mannose, GDP-fucose.

Optionally, the cell used to produce the saccharide is transformed to comprise and to express at least one nucleic acid sequence encoding a protein selected from the group consisting of lactose transporter, N-acetylneuraminic acid transporter, fucose transporter, glucose transporter, galactose transporter, transporter for a nucleotide-activated sugar wherein said transporter internalizes a to the medium added precursor for the synthesis of the saccharide of present invention.

The term “acceptor” as used herein refers to a mono-, di- or oligosaccharide, which can be modified by a glycosyltransferase. Examples of such acceptors comprise glucose, galactose, fructose, glycerol, sialic acid, fucose, mannose, maltose, sucrose, lactose, lacto-N-biose (LNB), N-acetyllactosamine (LacNAc), lacto-N-triose, lacto-N-tetraose (LNT), lacto-N-neotetraose (LNnT), lacto-N-pentaose (LNP), lacto-N-neopentaose, para lacto-N-pentaose, para lacto-N-neopentaose, lacto-N-novopentaose I, lacto-N-hexaose (LNH), lacto-N-neohexaose (LNnH), para lacto-N-neohexaose (pLNnH), para lacto-N-hexaose (pLNH), lacto-N-heptaose, lacto-N-neoheptaose, para lacto-N-neoheptaose, para lacto-N-heptaose, lacto-N-octaose (LNO), lacto-N-neooctaose, iso lacto-N-octaose, para lacto-N-octaose, iso lacto-N-neooctaose, novo lacto-N-neooctaose, para lacto-N-neooctaose, iso lacto-N-nonaose, novo lacto-N-nonaose, lacto-N-nonaose, lacto-N-decaose, iso lacto-N-decaose, novo lacto-N-decaose, lacto-N-neodecaose, and oligosaccharide containing 1 or more N-acetyllactosamine units and/or 1 or more lacto-N-biose units or an intermediate into oligosaccharide, fucosylated and sialylated versions thereof, ceramide, N-acylated sphingoid, glucosylceramide, lactosylceramide, sphingosine, phytosphingosine, sphingosine synthons, peptide backbones with beta-GlcNAc-Asn residues, glycoproteins with terminal GlcNAc and Gal residues, immunoglobulins.

The term “fermentation broth” comprises the culture medium wherein the cell is cultivated, or fermented, medium components, the cell itself, biomass and a saccharide that is produced by the cell in whole broth, i.e., inside (intracellularly) as well as outside (extracellularly) of the cell.

The term “biomass” as used herein refers to the suspended, precipitated or insoluble materials originating from fermentation cells, like intact cells, disrupted cells, cell fragments, cell walls, phospholipids, cell membranes, proteins, protein fragments, polysaccharides, polynucleotides and other large organic compounds produced by the cell. The biomass may be in suspension and/or in solution. Biomass as used herein is also to be understood to be non-complex biomass, typically of low or no organisation of cells into peculiar or complex structures. Non-limiting examples are individual cells, cell pairs, cell lumps, oligocellular or multicellular structures, cell layers, biofilms. The non-complex biomass may be of a three-dimensional structure, like e.g., cells forming a layer or a biofilm or attached to a surface of a reactor or incubator. The terms “reactor” and “incubator” refer to the recipient filled with the fermentation broth. Examples of reactors and incubators comprise but are not limited to microfluidic devices, well plates, tubes, shake flasks, fermenters, bioreactors and process vessels. Said reactor and incubator can each vary from lab-scale dimensions to large-scale industrial dimensions.

In contrast to non-complex biomass, complex biomass is to be understood to be of a complex structure by nature, often a complex three-dimensional structure and comprise many hundreds, thousands, ten-thousands, but more typically hundreds of thousands or millions or more of cellular structures in a complex organisation, often of various cell types with different specialisations. Non-limiting examples are higher plants or animals with a body visible with the naked eye, organs and tissues, including bone and meat or plant parts like fruit, vegetables, straw, sugarcane bagasse, hay, wood, timber. Complex biomass may be the source of non-complex biomass, for example cell lines are typically derived from a tissue or organ but do not maintain the complex structure in cultivation.

The terms “cell dry weight” or “CDW” as used herein refer to the grams of dry weight of biomass per liter of sample after removal of moisture. The procedure for measuring the CDW is disclosed in the working examples below.

As used herein, the term “cell productivity index (CPI)” refers to the mass of the saccharide produced by the cells divided by the mass of the cells produced in the culture.

The term “any process stream” is to be understood as any solution that occurs or that is used or that is created at any step throughout the purification method of present invention. Examples of said process streams comprise but are not limited to an inlet solution, outlet solution, influent, effluent, eluent, eluate, retentate, permeate, flow, waste solution, buffer, solvent, alcohol, acid, base, lysate, filtrate, extract.

The term “pure water flux” is defined as the volume of purified water like e.g., distilled water, RO water, that passes through a membrane per unit time and per unit area under specified conditions, like e.g., at 43-45° C., 5 bar and a constant crossflow of 1000 L/h, for at least 10 minutes.

The “rejection factor” of a membrane for an ion (in %) is calculated as (1−κ_{P}/κ_{F})·100, wherein κ_{P} is the conductivity of the ion in the permeate and κ_{F} is the conductivity of the ion in the retentate.

The terms “permeate” and “filtrate” are used interchangeably and refer to the fraction that pass through a membrane used in a filtration and/or diafiltration step. The terms “retentate” and “concentrate” are used interchangeably and refer to the fraction that does not pass through a membrane used in a filtration and/or diafiltration step.

The term “diafiltration” as used herein refers to a filtration process wherein water is added to the retentate while the volume of said retentate remains constant. Preferably, the flow rate of the diafiltration water is equal to the filtrate flow.

The term “fouling cake” as used herein refers to the deposition and accumulation of feed components like e.g., biomass, cell debris, antifoam, peptides, lipides, particles, solutes, macromolecules, on the surface and/or in the pores of a membrane during filtration and/or diafiltration.

The term “purified” refers to material that is substantially or essentially free from components that interfere with the activity of the biological molecule. For cells, saccharides, nucleic acids, and polypeptides, the term “purified” refers to material that is substantially or essentially free from components that normally accompany the material as found in its native state. Typically, purified saccharides, oligosaccharides, proteins or nucleic acids of the invention are at least about 50%, 55%, 60%, 65%, 70%, 75%, 80% or 85% pure, usually at least about 90%, 91%, 92%, 93%, 94%, 95%, 96%, 97%, 98%, or 99.0% pure as measured by band intensity on a silver-stained gel or other method for determining purity. Purity or homogeneity can be indicated by a number of means well known in the art, such as polyacrylamide gel electrophoresis of a protein or nucleic acid sample, followed by visualization upon staining. For certain purposes high resolution will be needed and HPLC or a similar means for purification utilized. For di- and oligosaccharides, purity can be determined using methods such as but not limited to thin layer chromatography, gas chromatography, NMR, HPLC, capillary electrophoresis or mass spectroscopy. Further herein, the terms “contaminants” and “impurities” preferably mean particulates, cells, cell components, metabolites, cell debris, proteins, peptides, amino acids, nucleic acids, glycolipids and/or endotoxins which can be present in an aqueous medium like e.g., a fermentation broth.

The term “clarifying” as used herein refers to the act of treating an aqueous medium like e.g., a fermentation broth to remove suspended particulates and contaminants from the production process, like e.g. cells, cell components, insoluble metabolites and debris, that could interfere with the eventual purification of the saccharide or the saccharide mixture. Such treatment can be carried out in a conventional manner by centrifugation, flocculation, flocculation with optional ultrasonic treatment, gravity filtration, microfiltration, foam separation or vacuum filtration (e.g., through a ceramic filter which can include a Celite™ filter aid).

The terms “protein-free saccharide solution” as used herein means a saccharide solution from a fermentation broth, which has been treated to remove substantially all the proteins, as well as any related impurities, such as amino acids, peptides, peptide fragments, endotoxins, glycolipids, RNA and DNA, from the process that could interfere with the eventual purification of the saccharide solution from the process. Such removal of proteins, preferably substantially all proteins, can be accomplished, e.g., in a conventional manner by ion exchange chromatography, affinity chromatography, ultrafiltration, and size exclusion chromatography. The terms “purification of a saccharide solution from a fermentation broth” according to the present invention mean harvesting, collecting or retrieving the saccharide solution from the cells and/or the medium of its growth.

A “purified saccharide solution” comprises one saccharide or a mixture of saccharides dissolved in an aqueous medium. An aqueous medium is a solvent comprising water. In some embodiments, the aqueous medium is pure water. In other embodiments, the medium comprises water with a trace amount of one or more organic solvents. In some such embodiments, the medium comprises less than 1%-wt. (percent by weight) organic solvent. In some embodiments, the medium comprises less than 0.1%-wt. organic solvent. In some embodiments, the medium comprises less than 0.01%-wt. organic solvent. In some embodiments, the medium comprises less than 0.001%-wt. organic solvent. In some embodiments, the medium comprises less than 0.0001%-wt. organic solvent.

In some embodiments, the saccharide solution comprises a trace amount of one or more organic solvents. In some such embodiments, the purified saccharide solution comprises less than 1%-wt. organic solvent. In some embodiments, the purified saccharide solution comprises less than 0.1%-wt. organic solvent. In some embodiments, the purified saccharide solution comprises less than 0.01%-wt. organic solvent. In some embodiments, the purified saccharide solution comprises less than 0.001%-wt. organic solvent. In some embodiments, the purified saccharide solution comprises less than 0.0001%-wt. organic solvent. As used herein a “Brix value” indicates the sugar content of an aqueous solution. A Brix value can be expressed as a percentage (percent Brix) or as “degrees Brix”. Strictly, a Brix value is the percentage by weight of sucrose in a pure water solution, and so does not apply to solutions comprising other solutes and/or solvents. However, a Brix value is simple to measure, and, therefore, is commonly used in the art as an approximation of the total saccharide content of sugar solutions other than pure sucrose solutions. As used herein, the “Brix value” indicates the combined sugar content of the aqueous solution, when the purified saccharide solution comprises two or more different saccharides. Techniques for measuring a Brix value are well known in the art. Dissolution of sugar in an aqueous solution changes the refractive index of the solution. Accordingly, an appropriately calibrated refractometer can be used to measure a Brix value of a solution. Alternatively, the density of a solution may be measured and converted to a Brix value. A digital density meter can perform this measurement and conversion automatically, or a hydrometer or pycnometer may be used.

The terms “dry solid” and “dry matter” as used herein are used interchangeably and are further described in Example 1.

The ash content is a measure of the total amount of minerals present within a food or ingredients such as saccharides, whereas the mineral content is a measure of the amount of specific inorganic components present within a food, such as Ca²⁺, Na⁺, K⁺, Mg²⁺, phosphate, sulphate and Cl⁻. Ash is the inorganic residue remaining after the water and organic matter have been removed by heating in the presence of oxidizing agents, which provides a measure of the total amount of minerals within a food. Analytical techniques for providing information about the total mineral content are based on the fact that the minerals (the analyte) can be distinguished from all the other components (the matrix) within a food or ingredient in some measurable way. The most widely used methods are based on the fact that minerals are not destroyed by heating, and that they have a low volatility compared to other food components. The three main types of analytical procedure used to determine the ash content of foods are based on this principle: dry ashing, wet ashing and low temperature plasma dry ashing. The method chosen for a particular analysis depends on the reason for carrying out the analysis, the type of food or ingredient analyzed and the equipment available. Ashing may also be used as the first step in preparing samples for analysis of specific minerals, by atomic spectroscopy or the various traditional methods described below. For the sample preparation a sample whose composition represents that of the ingredient is selected to ensure that its composition does not change significantly prior to analysis. For instance, a dry saccharide sample is generally hygroscopic, and the selected sample should be kept under dry conditions avoiding the absorption of water. Typically, samples of 1-10 gram are used in the analysis of ash content. Solid ingredients are finely ground and then carefully mixed to facilitate the choice of a representative sample. Before carrying out an ash analysis, samples that are high in moisture or in solution are generally dried to prevent spattering during ashing. Other possible problems include contamination of samples by minerals in grinders, glassware or crucibles which come into contact with the sample during the analysis. For the same reason, deionized water is used when preparing samples and the same is used in the blank sample. Dry ashing procedures use a high temperature muffle furnace capable of maintaining temperatures of between 50° and 600° C. Water and other volatile materials are vaporized and organic substances are burned in the presence of the oxygen in air to CO₂, H₂O and N₂. Most minerals are converted to oxides, sulphates, phosphates, chlorides or silicates. Although most minerals have fairly low volatility at these high temperatures, some are volatile and may be partially lost, e.g., iron, lead and mercury, for these minerals ICP-MS analysis of the product is more appropriate for quantification.

### DETAILED DESCRIPTION OF THE INVENTION

According to a first aspect, the present invention provides a method for the purification of saccharide from a fermentation broth comprising said saccharide and biomass. The method comprises filtration of said fermentation broth on a membrane and subsequent diafiltration on said membrane under conditions permissive to produce a retentate comprising said biomass and a permeate comprising said saccharide, wherein said membrane comprises a molecular weight cut-off ranging from 0.3 to 5 kDa, a pore size ranging from 0.001 to 0.01 μm and/or a monovalent ion rejection ranging from 1 to 50%. In a preferred embodiment, the membrane comprises a molecular weight cut-off ranging from 0.5 to 4 kDa. In a more preferred embodiment, the membrane comprises a molecular weight cut-off ranging from 1 to 3.5 kDa. In an even more preferred embodiment, the membrane comprises a molecular weight cut-off ranging from 1.5 to 3 kDa. In another and/or additional preferred embodiment, the membrane comprises a monovalent ion rejection ranging from 5 to 40%. In a more preferred embodiment, the membrane comprises a monovalent ion rejection ranging from 10 to 20%.

More specifically, the present invention provides a method for the purification of saccharide from a fermentation broth comprising said saccharide and biomass, wherein said fermentation broth originates from a fermentation of a cell producing said saccharide and wherein said biomass consists essentially of or consists of intact cells, disrupted cells, cell fragments, cell walls, phospholipids, cell membranes, proteins, protein fragments, polysaccharides, polynucleotides and large organic compounds produced by the cell of said fermentation. The method comprises filtration of said fermentation broth on a membrane and subsequent diafiltration on said membrane under conditions permissive to collect (1) a retentate comprising, consisting of or consisting essentially of said biomass and (2) a permeate comprising essentially all or all of said saccharide. In other words, said filtration and subsequent diafiltration on the same membrane of said fermentation broth comprising said saccharide and biomass results in the separation of said biomass in the retentate from essentially all or all of said saccharide in the permeate. Herein, the permeate is an aqueous solution comprising essentially all or all of said saccharide. Furthermore, said membrane comprises a molecular weight cut-off ranging from 0.3 to 5 kDa, a pore size ranging from 0.001 to 0.01 μm and/or a monovalent ion rejection ranging from 1 to 50%. In a preferred embodiment, the membrane comprises a molecular weight cut-off ranging from 0.5 to 4 kDa. In a more preferred embodiment, the membrane comprises a molecular weight cut-off ranging from 1 to 3.5 kDa. In an even more preferred embodiment, the membrane comprises a molecular weight cut-off ranging from 1.5 to 3 kDa. In another and/or additional preferred embodiment, the membrane comprises a monovalent ion rejection ranging from 5 to 40%. In a more preferred embodiment, the membrane comprises a monovalent ion rejection ranging from 10 to 20%.

The present invention concerns a process for the purification of a saccharide that is provided in a fermentation broth comprising said saccharide and biomass.

In a preferred embodiment, the saccharide is chosen from the list comprising monosaccharide; disaccharide; oligosaccharide; polysaccharide; neutral (non-charged) saccharide; negatively charged, preferably sialylated, saccharide; milk oligosaccharide; lactose; sucrose; glucose; glycerol; galactose; fucose; mannose; N-acetylglucosamine; N-acetylgalactosamine; lactosamine; lacto-N-biose; O-antigen; enterobacterial common antigen (ECA); the oligosaccharide repeats present in capsular polysaccharides; peptidoglycan; an amino-sugar; Lewis-type antigen oligosaccharide; an antigen of the human ABO blood group system; an animal oligosaccharide; a plant oligosaccharide; fucosylated oligosaccharide; sialylated oligosaccharide; N-acetylglucosamine containing neutral (non-charged) oligosaccharide; N-acetyllactosamine containing oligosaccharide; lacto-N-biose containing oligosaccharide; non-fucosylated neutral (non-charged) oligosaccharide; chitosan; chitosan comprising oligosaccharide; heparosan; chondroitin sulphate; glycosaminoglycan oligosaccharide; heparin; heparan sulphate; dermatan sulphate; hyaluronan; hyaluronic acid; and keratan sulphate.

In a more preferred embodiment, the saccharide is a monosaccharide as described herein. In another more preferred embodiment, the saccharide is a disaccharide as described herein. In another more preferred embodiment, the saccharide is an oligosaccharide as described herein. In another more preferred embodiment, the saccharide is a mammalian milk oligosaccharide (MMO) as described herein. In another more preferred embodiment, the saccharide is a human milk oligosaccharide (HMO) as described herein. In another more preferred embodiment, the saccharide is an animal oligosaccharide selected from the group consisting of N-glycans and O-glycans. In another more preferred embodiment, the saccharide is a plant oligosaccharide selected from the group consisting of N-glycans and O-glycans. In the context of present invention, N-glycans and O-glycans refer to the oligosaccharide structures as known by the person skilled in the art wherein said structures are not attached to a protein or a peptide. In another more preferred embodiment, the saccharide is a fucosylated oligosaccharide selected from the group comprising 2′-fucosyllactose (2′FL), 3-fucosyllactose (3FL), 4-fucosyllactose (4FL), 6-fucosyllactose (6FL), 2′,3-difucosyllactose (diFL), lacto-N-fucopentaose I, lacto-N-neofucopentaose I, lacto-N-fucopentaose II, lacto-N-fucopentaose III, lacto-N-fucopentaose V, lacto-N-fucopentaose VI, lacto-N-neofucopentaose V, lacto-N-difucohexaose I, lacto-N-difucohexaose II, difucosyl-lacto-N-hexaose and difucosyl-lacto-N-neohexaose. In another more preferred embodiment, the saccharide is a sialylated oligosaccharide selected from the group comprising 3′sialyllactose (3′SL), 6′sialyllactose (6′SL), sialyllacto-N-tetraose a (LSTa), sialyllacto-N-tetraose b (LSTb), sialyllacto-N-tetraose c (LSTc), sialyllacto-N-tetraose d (LSTd), disialyllacto-N-tetraose, disialyllacto-N-neotetraose, monosialyllacto-N-hexaose, disialyllacto-N-hexaose I, disialyllacto-N-hexaose II, monosialyllacto-N-neohexaose I, monosialyllacto-N-neohexaose II, disialyllacto-N-neohexaose, 3′-sialyl-3-fucosyllactose, fucodisialyllacto-N-hexaose, disialomonofucosyllacto-N-neohexaose, sialyllacto-N-fucohexaose II, disialyllacto-N-fucopentaose II and monofucosyldisialyllacto-N-tetraose. In another more preferred embodiment, the saccharide is an N-acetylglucosamine containing neutral (non-charged) oligosaccharide selected from the group comprising lacto-N-triose II (LN3), lacto-N-tetraose (LNT), lacto-N-neotetraose (LNnT), 6′-galactosyllactose, 3′-galactosyllactose, lacto-N-hexaose, lacto-N-neohexaose, para-lacto-N-hexaose and para-lacto-N-neohexaose.

In an even more preferred embodiment, the saccharide is chosen from the list comprising glucose; glycerol; galactose; fucose; mannose; N-acetylglucosamine; N-acetylgalactosamine; N-acetylmannosamine; xylose; rhamnose; glucuronate; galacturonate; lactose; sucrose; lactosamine; lacto-N-biose; maltose; raffinose; Fucα1-2Galβ1-3GlcNAc; Gal(31-3[Fucα1-4]GlcNAc; Fucα1-2Galβ1-3[Fucα1-4]GlcNAc; Neu5Aca2-3Galβ1-3[Fucα1-4]GlcNAc; Fucα1-2Galβ1-4GlcNAc; Gal(31-4[Fucα1-3]GlcNAc; Fucα1-2Galβ1-4[Fucα1-3]GlcNAc; Neu5Aca2-3Galβ1-4[Fucα1-3]GlcNAc; 2′-fucosyllactose (2′FL); 3-fucosyllactose (3FL); 4-fucosyllactose (4FL); 6-fucosyllactose (6FL); 2′,3-difucosyllactose (diFL); lacto-N-fucopentaose I; lacto-N-neofucopentaose I; lacto-N-fucopentaose II; lacto-N-fucopentaose III; lacto-N-fucopentaose V; lacto-N-fucopentaose VI; lacto-N-neofucopentaose V; lacto-N-difucohexaose I; lacto-N-difucohexaose II; difucosyl-lacto-N-hexaose; difucosyl-lacto-N-neohexaose; lacto-N-triose II (LN3); lacto-N-tetraose (LNT); lacto-N-neotetraose (LNnT); 6′-galactosyllactose; 3′-galactosyllactose; lacto-N-hexaose; lacto-N-neohexaose; para-lacto-N-hexaose; para-lacto-N-neohexaose; 3′sialyllactose (3′SL); 6′sialyllactose (6′SL); sialyllacto-N-tetraose a (LSTa); sialyllacto-N-tetraose b (LSTb); sialyllacto-N-tetraose c (LSTc); sialyllacto-N-tetraose d (LSTd); disialyllacto-N-tetraose; disialyllacto-N-neotetraose; monosialyllacto-N-hexaose; disialyllacto-N-hexaose I; disialyllacto-N-hexaose II; monosialyllacto-N-neohexaose I; monosialyllacto-N-neohexaose II; disialyllacto-N-neohexaose; 3′-sialyl-3-fucosyllactose; fucodisialyllacto-N-hexaose; disialomonofucosyllacto-N-neohexaose; sialyllacto-N-fucohexaose II; disialyllacto-N-fucopentaose II and monofucosyldisialyllacto-N-tetraose.

The saccharide in the context of the present invention is preferably in free form, i.e., the saccharide does not contain any protective group.

In an embodiment of present invention, the saccharide to be purified is present in a fermentation broth. According to a preferred embodiment, the fermentation broth originates from a fermentation of a cell producing the saccharide to be purified. Within the context of the present invention, said fermentation broth is produced by incubation in a reactor or incubator as defined herein. Said reactor or incubator can vary from small-scale dimensions (lab-scale) to large-scale dimensions (industrial set-up).

In another embodiment, the fermentation broth comprising a saccharide to be purified by a method of present invention also comprises biomass. In a preferred embodiment, the fermentation broth comprises a saccharide to be purified by a method of present invention, biomass and medium components. In another and/or additional preferred embodiment, the fermentation broth further comprises antifoam and/or proteins. In another and/or additional preferred embodiment, the fermentation broth further comprises at least 0.01% antifoam (v/v). At least 0.01% antifoam (v/v) is to be understood as 0.01% antifoam (v/v) or more than 0.01% antifoam (v/v), comprising 0.02% antifoam (v/v), 0.03% antifoam (v/v), 0.04% antifoam (v/v), 0.05% antifoam (v/v), 0.06% antifoam (v/v), 0.07% antifoam (v/v), 0.08% antifoam (v/v), 0.09% antifoam (v/v), 0.1% antifoam (v/v) or more than 0.1% antifoam (v/v). In a more preferred embodiment, the fermentation broth further comprises at least 0.1% antifoam (v/v). At least 0.1% antifoam (v/v) is to be understood as 0.1% antifoam (v/v) or more than 0.1% antifoam (v/v), comprising 0.2% antifoam (v/v), 0.3% antifoam (v/v), 0.4% antifoam (v/v), 0.5% antifoam (v/v), 0.6% antifoam (v/v), 0.7% antifoam (v/v), 0.8% antifoam (v/v), 0.9% antifoam (v/v), 1% antifoam (v/v) or more than 1% antifoam (v/v). In an even more preferred embodiment, the fermentation broth further comprises at least 1% antifoam (v/v). At least 1% antifoam (v/v) is to be understood as 1% antifoam (v/v) or more than 1% antifoam (v/v), comprising 1.1% antifoam (v/v), 1.2% antifoam (v/v), 1.3% antifoam (v/v), 1.4% antifoam (v/v), 1.5% antifoam (v/v), 1.6% antifoam (v/v), 1.7% antifoam (v/v), 1.8% antifoam (v/v), 1.9% antifoam (v/v), 2% antifoam (v/v) or more than 2% antifoam (v/v).

In another and/or additional preferred embodiment, the purity of said saccharide in said fermentation broth is <70%, <60%, <50%, <40%, <30%, <20%, <10% on total dry solid before purification by said method.

In another and/or additional preferred embodiment, the saccharide is accompanied in said fermentation broth by sialic acid as defined herein; ashes, preferably, said ashes comprise sulphates and phosphates; one or more monosaccharide(s) like e.g., fucose (Fuc), galactose (Gal), glucose (Glc), N-acetylglucosamine (GlcNAc), N-acetylgalactosamine (GalNAc), mannose (Man), N-acetylmannosamine (ManNAc); one or more activated monosaccharide(s) like e.g., UDP-N-acetylglucosamine (UDP-GlcNAc), UDP-N-acetylgalactosamine (UDP-GalNAc), UDP-N-acetylmannosamine (UDP-ManNAc), UDP-glucose (UDP-Glc), UDP-galactose (UDP-Gal), GDP-mannose (GDP-Man), GDP-fucose, (GDP-Fuc), UDP-glucuronate, UDP-galacturonate, CMP-sialic acid (CMP-Neu5Ac), CMP-Neu4Ac, CMP-Neu5Ac9N₃, CMP-Neu4,5Ac₂, CMP-Neu5,7Ac₂, CMP-Neu5,9Ac₂, CMP-Neu5,7(8,9)Ac₂, CMP-N-glycolylneuraminic acid (CMP-Neu5Gc); one or more phosphorylated monosaccharide(s) like e.g., fructose-6-phosphate, glucose-6-phosphate, glucose-1-phosphate, glucosamine-6-phosphate, N-acetylglucosamine-6-phosphate, N-acetylglucosamine-1-phosphate, galactose-1-phosphate, N-acetylmannosamine-6-phosphate, mannose-6-phosphate, mannose-1-phosphate, fructose-1-phosphate, fructose-1,6-bisphosphate, glycerol-3-phosphate, glyceraldehyde-3-phosphate, dihydroxyacetone-phosphate; and one or more other saccharide(s) like e.g., a disaccharide, oligosaccharide, polysaccharide, neutral (non-charged) saccharide, negatively charged, preferably sialylated, saccharide, sucrose, lactose (Gal-β1,4-Glc), lacto-N-biose (Gal-β1,3-GlcNAc), N-acetyllactosamine (Gal-β1,4-GlcNAc), a neutral (non-charged) oligosaccharide, a negatively charged oligosaccharide, a milk oligosaccharide, preferably a mammalian milk oligosaccharide (MMO), more preferably a human milk oligosaccharide (HMO); fucosylated oligosaccharide; sialylated oligosaccharide; neutral (non-charged) non-fucosylated oligosaccharide; O-antigen; enterobacterial common antigen (ECA); the oligosaccharide repeats present in capsular polysaccharides; peptidoglycan; an amino-sugar; Lewis-type antigen oligosaccharide; an antigen of the human ABO blood group system; an animal oligosaccharide, preferably selected from the group consisting of N-glycans and O-glycans; a plant oligosaccharide, preferably selected from the group consisting of N-glycans and O-glycans; 2′-fucosyllactose (2′FL), 3-fucosyllactose (3FL), 4-fucosyllactose (4FL), 6-fucosyllactose (6FL), 2′,3-difucosyllactose (diFL), lacto-N-fucopentaose I, lacto-N-neofucopentaose I, lacto-N-fucopentaose II, lacto-N-fucopentaose III, lacto-N-fucopentaose V, lacto-N-fucopentaose VI, lacto-N-neofucopentaose V, lacto-N-difucohexaose I, lacto-N-difucohexaose II, difucosyl-lacto-N-hexaose, difucosyl-lacto-N-neohexaose, 3′sialyllactose (3′SL), 6′sialyllactose (6′SL), sialyllacto-N-tetraose a (LSTa), sialyllacto-N-tetraose b (LSTb), sialyllacto-N-tetraose c (LSTc), sialyllacto-N-tetraose d (LSTd), disialyllacto-N-tetraose, disialyllacto-N-neotetraose, monosialyllacto-N-hexaose, disialyllacto-N-hexaose I, disialyllacto-N-hexaose II, monosialyllacto-N-neohexaose I, monosialyllacto-N-neohexaose II, disialyllacto-N-neohexaose, 3′-sialyl-3-fucosyllactose, fucodisialyllacto-N-hexaose, disialomonofucosyllacto-N-neohexaose, sialyllacto-N-fucohexaose II, disialyllacto-N-fucopentaose II, monofucosyldisialyllacto-N-tetraose, lacto-N-triose II (LN3), lacto-N-tetraose (LNT), lacto-N-neotetraose (LNnT), 6′-galactosyllactose, 3′-galactosyllactose, lacto-N-hexaose, lacto-N-neohexaose, para-lacto-N-hexaose, para-lacto-N-neohexaose; N-acetylglucosamine containing oligosaccharide, N-acetylglucosamine containing neutral (non-charged) oligosaccharide, N-acetyllactosamine containing oligosaccharide; lacto-N-biose containing oligosaccharide; non-fucosylated neutral (non-charged) oligosaccharide; chitosan; chitosan comprising oligosaccharide; heparosan; glycosaminoglycan oligosaccharide; heparin; heparan sulphate; chondroitin sulphate; dermatan sulphate; hyaluronan; hyaluronic acid; and keratan sulphate.

In a preferred embodiment, the saccharide to be purified by a method of present invention is produced by a cell that is fermented. In another preferred embodiment of present invention, the cell is a prokaryotic cell. In a more preferred embodiment, the cell is selected from the group consisting of yeast cells, bacterial cells, archaebacterial cells and fungal cells. In another preferred embodiment, the cell is a bacterium, fungus, yeast or a protozoan cell. The latter bacterium preferably belongs to the phylum of the Proteobacteria or the phylum of the Firmicutes or the phylum of the Cyanobacteria or the phylum Deinococcus-Thermus or the phylum of Actinobacteria. The latter bacterium belonging to the phylum Proteobacteria belongs preferably to the family Enterobacteriaceae, preferably to the species Escherichia coli. The latter bacterium preferably relates to any strain belonging to the species Escherichia coli such as but not limited to Escherichia coli B, Escherichia coli C, Escherichia coli W, Escherichia coli K12, Escherichia coli Nissle. More specifically, the latter term relates to cultivated Escherichia coli strains—designated as E. coli K12 strains—which are well-adapted to the laboratory environment, and, unlike wild type strains, have lost their ability to thrive in the intestine. Well-known examples of the E. coli K12 strains are K12 Wild type, W3110, MG1655, M182, MC1000, MC1060, MC1061, MC4100, JM101, NZN111 and AA200. Hence, the present invention specifically relates to a mutated and/or transformed Escherichia coli cell or strain as indicated above wherein said E. coli strain is a K12 strain. More preferably, the Escherichia coli K12 strain is E. coli MG1655. The latter bacterium belonging to the phylum Firmicutes belongs preferably to the Bacilli, preferably Lactobacilliales, with members such as Lactobacillus lactis, Leuconostoc mesenteroides, or Bacillales with members such as from the genus Bacillus, such as Bacillus subtilis or, B. amyloliquefaciens. The latter Bacterium belonging to the phylum Actinobacteria, preferably belonging to the family of the Corynebacteriaceae, with members Corynebacterium glutamicum or C. afermentans, or belonging to the family of the Streptomycetaceae with members Streptomyces griseus or S. fradiae. The latter bacterium belonging to the phylum Proteobacteria, preferably belonging to the family of the Vibrionaceae, with member Vibrio natriegens. The latter yeast preferably belongs to the phylum of the Ascomycota or the phylum of the Basidiomycota or the phylum of the Deuteromycota or the phylum of the Zygomycetes. The latter yeast belongs preferably to the genus Saccharomyces (with members like e.g. Saccharomyces cerevisiae, S. bayanus, S. boulardii), Zygosaccharomyces, Pichia (with members like e.g. Pichia pastoris, P. anomala, P. kluyveri), Komagataella, Hansenula, Kluyveromyces (with members like e.g. Kluyveromyces lactis, K. marxianus, K. thermotolerans), Debaromyces, Candida, Schizosaccharomyces, Schwanniomyces, Torulaspora, Yarrowia (like e.g. Yarrowia lipolytica) or Starmerella (like e.g. Starmerella bombicola). The latter yeast is preferably selected from Pichia pastoris, Yarrowia lipolytica, Saccharomyces cerevisiae, Kluyveromyces lactis, Hansenula polymorpha, Kluyveromyces marxianus, Pichia methanolica, Pichia stipites, Candida boidinii, Schizosaccharomyces pombe, Schwanniomyces occidentalis, Torulaspora delbrueckii, Zygosaccharomyces rouxii, and Zygosaccharomyces bailii. The latter fungus belongs preferably to the genus Rhizopus, Dictyostelium, Penicillium, Mucor or Aspergillus. The latter protozoan cell preferably is a Leishmania tarentolae cell.

In another and/or additional preferred embodiment, the cell is an E. coli or yeast with a lactose permease positive phenotype, preferably wherein said lactose permease is coded by the gene LacY or LAC12, respectively.

In another and/or additional more preferred embodiment, the cell is a metabolically engineered cell. In another and/or additional more preferred embodiment, the cell has been metabolically engineered to produce any one or more compound(s) that is/are not (a) saccharide(s). In another and/or additional more preferred embodiment, the cell has been metabolically engineered to produce a saccharide. In an even more preferred embodiment, the cell has been metabolically engineered to produce two or more saccharides.

In another and/or additional preferred embodiment, the cell produces a saccharide and any one or more of sialic acid as defined herein; one or more monosaccharide(s); one or more activated monosaccharide(s); one or more phosphorylated monosaccharide(s) and/or one or more other saccharide(s), as described herein. In another and/or additional preferred embodiment, the cell has been metabolically engineered to produce a saccharide and any one or more of sialic acid as defined herein; one or more monosaccharide(s), one or more activated monosaccharide(s), one or more phosphorylated monosaccharide(s) and/or one or more other saccharide(s) as described herein.

In another and/or additional preferred embodiment, the fermentation broth is a cell cultivation using at least one cell that has been metabolically engineered to produce said saccharide and one or more of i) sialic acid, ii) one or more monosaccharide(s), iii) one or more activated monosaccharide(s), iv) one or more phosphorylated monosaccharide(s) and/or v) one or more other saccharides.

In a more preferred embodiment, the cell comprises a sialyation pathway. A sialylation pathway is a biochemical pathway consisting of at least one of the enzymes and their respective genes chosen from the list comprising an L-glutamine-D-fructose-6-phosphate aminotransferase, a phosphoglucosamine mutase, an N-acetylglucosamine-6-P deacetylase, an N-acylglucosamine 2-epimerase, a UDP-N-acetylglucosamine 2-epimerase, an N-acetylmannosamine-6-phosphate 2-epimerase, a UDP-GlcNAc 2-epimerase/kinase, a glucosamine 6-phosphate N-acetyltransferase, an N-acetylglucosamine-6-phosphate phosphatase, a phosphoacetylglucosamine mutase, an N-acetylglucosamine 1-phosphate uridylyltransferase, a glucosamine-1-phosphate acetyltransferase, an Neu5Ac synthase, an N-acetylneuraminate lyase, an N-acylneuraminate-9-phosphate synthase, an N-acylneuraminate-9-phosphatase, a sialic acid transporter, a cytidine monophosphate (CMP) kinase and a CMP-sialic acid synthase, combined with a sialyltransferase leading to any one or more of a 2,3; a 2,6 and/or a 2,8 sialylated oligosaccharides.

In an even more preferred embodiment, the cell is metabolically engineered to comprise a sialylation pathway. In another even more preferred embodiment, the cell has been metabolically engineered to comprise a sialylation pathway wherein any one or more of the genes chosen from the list comprising L-glutamine-D-fructose-6-phosphate aminotransferase, phosphoglucosamine mutase, N-acetylglucosamine-6-P deacetylase, N-acylglucosamine 2-epimerase, UDP-N-acetylglucosamine 2-epimerase, N-acetylmannosamine-6-phosphate 2-epimerase, UDP-GlcNAc 2-epimerase/kinase, glucosamine 6-phosphate N-acetyltransferase, N-acetylglucosamine-6-phosphate phosphatase, phosphoacetylglucosamine mutase, N-acetylglucosamine 1-phosphate uridylyltransferase, glucosamine-1-phosphate acetyltransferase, Neu5Ac synthase, N-acetylneuraminate lyase, N-acylneuraminate-9-phosphate synthase, N-acylneuraminate-9-phosphatase, sialic acid transporter, CMP kinase, CMP-sialic acid synthase and sialyltransferase has/have a modified and/or enhanced expression.

In another and/or additional preferred embodiment, the cell comprises a fucosylation pathway. A fucosylation pathway is a biochemical pathway consisting of at least one of the enzymes and their respective genes chosen from the list comprising mannose-6-phosphate isomerase, phosphomannomutase, mannose-1-phosphate guanylyltransferase, GDP-mannose 4,6-dehydratase, GDP-L-fucose synthase, fucose permease, fucose kinase, fucose-1-phosphate guanylyltransferase combined with a fucosyltransferase leading to a 1,2; a 1,3; a 1,4 and/or a 1,6 fucosylated oligosaccharides.

In a more preferred additional and/or alternative embodiment, the cell is metabolically engineered to comprise a fucosylation pathway. In another even more preferred additional and/or alternative embodiment, the cell has been metabolically engineered to comprise a fucosylation pathway wherein any one or more of the genes chosen from the list comprising mannose-6-phosphate isomerase, phosphomannomutase, mannose-1-phosphate guanylyltransferase, GDP-mannose 4,6-dehydratase, GDP-L-fucose synthase, fucose permease, fucose kinase, fucose-1-phosphate guanylyltransferase and fucosyltransferase has/have a modified and/or enhanced expression.

In another and/or additional preferred embodiment, the cell comprises a galactosylation pathway. A galactosylation pathway is a biochemical pathway consisting of at least one of the enzymes and their respective genes chosen from the list comprising galactose-1-epimerase, galactokinase, glucokinase, galactose-1-phosphate uridylyltransferase, UDP-glucose 4-epimerase, glucose-1-phosphate uridylyltransferase, phosphoglucomutase combined with a galactosyltransferase leading to a galactosylated compound comprising a mono-, di-, or oligosaccharide having an alpha or beta bound galactose on any one or more of the 2, 3, 4 and 6 hydroxyl group of said mono-, di-, or oligosaccharide.

In a more preferred additional and/or alternative embodiment, the cell is metabolically engineered to comprise a galactosylation pathway. In another even more preferred additional and/or alternative embodiment, the cell has been metabolically engineered to comprise a galactosylation pathway wherein any one or more of the genes chosen from the list comprising galactose-1-epimerase, galactokinase, glucokinase, galactose-1-phosphate uridylyltransferase, UDP-glucose 4-epimerase, glucose-1-phosphate uridylyltransferase, phosphoglucomutase and galactosyltransferase has/have a modified and/or enhanced expression.

In another and/or additional preferred embodiment, the cell comprises an ‘N-acetylglucosaminylation’ pathway. An N-acetylglucosaminylation pathway is a biochemical pathway consisting of at least one of the enzymes and their respective genes chosen from the list comprising L-glutamine-D-fructose-6-phosphate aminotransferase, N-acetylglucosamine-6-phosphate deacetylase, phosphoglucosamine mutase, N-acetylglucosamine-1-phosphate uridylyltransferase, glucosamine-1-phosphate acetyltransferase combined with a glycosyltransferase leading to a GlcNAc-modified compound comprising a mono-, di-, or oligosaccharide having an alpha or beta bound N-acetylglucosamine (GlcNAc) on any one or more of the 3, 4 and 6 hydroxyl group of said mono-, di- or oligosaccharide.

In a more preferred additional and/or alternative embodiment, the cell is metabolically engineered to comprise an N-acetylglucosaminylation pathway. In another even more preferred additional and/or alternative embodiment, the cell has been metabolically engineered to comprise an N-acetylglucosaminylation pathway wherein any one or more of the genes chosen from the list comprising L-glutamine-D-fructose-6-phosphate aminotransferase, N-acetylglucosamine-6-phosphate deacetylase, phosphoglucosamine mutase, N-acetylglucosamine-1-phosphate uridylyltransferase, glucosamine-1-phosphate acetyltransferase and a glycosyltransferase transferring GlcNAc has/have a modified and/or enhanced expression.

In another and/or additional preferred embodiment, the cell comprises an ‘N-acetylgalactosaminylation’ pathway. An N-acetylgalactosaminylation pathway is a biochemical pathway consisting of at least one of the enzymes and their respective genes chosen from the list comprising L-glutamine-D-fructose-6-phosphate aminotransferase, phosphoglucosamine mutase, N-acetylglucosamine 1-phosphate uridylyltransferase, glucosamine-1-phosphate acetyltransferase, UDP-N-acetylglucosamine 4-epimerase, UDP-glucose 4-epimerase, N-acetylgalactosamine kinase and/or UDP-N-acetylgalactosamine pyrophosphorylase combined with a glycosyltransferase leading to a GalNAc-modified compound comprising a mono-, di- or oligosaccharide having an alpha or beta bound N-acetylgalactosamine on said mono-, di- or oligosaccharide.

In a more preferred additional and/or alternative embodiment, the cell is metabolically engineered to comprise an N-acetylgalactosaminylation pathway. In another even more preferred additional and/or alternative embodiment, the cell has been metabolically engineered to comprise an N-acetylgalactosaminylation pathway wherein any one or more of the genes chosen from the list comprising L-glutamine-D-fructose-6-phosphate aminotransferase, phosphoglucosamine mutase, N-acetylglucosamine 1-phosphate uridylyltransferase, glucosamine-1-phosphate acetyltransferase, UDP-N-acetylglucosamine 4-epimerase, UDP-glucose 4-epimerase, N-acetylgalactosamine kinase and/or UDP-N-acetylgalactosamine pyrophosphorylase and a glycosyltransferase transferring GalNAc has/have a modified and/or enhanced expression.

In another and/or additional preferred embodiment, the cell comprises a ‘mannosylation’ pathway. A mannosylation pathway is a biochemical pathway consisting of at least one of the enzymes and their respective genes chosen from the list comprising mannose-6-phosphate isomerase, phosphomannomutase and/or mannose-1-phosphate guanylyltransferase combined with a mannosyltransferase leading to a mannosylated compound comprising a mono-, di- or oligosaccharide having an alpha or beta bound mannose on said mono-, di- or oligosaccharide.

In a more preferred additional and/or alternative embodiment, the cell is metabolically engineered to comprise a mannosylation pathway. In another even more preferred additional and/or alternative embodiment, the cell has been metabolically engineered to comprise a mannosylation pathway wherein any one or more of the genes chosen from the list comprising mannose-6-phosphate isomerase, phosphomannomutase and/or mannose-1-phosphate guanylyltransferase and mannosyltransferase has/have a modified and/or enhanced expression.

In another and/or additional preferred embodiment, the cell comprises an ‘N-acetylmannosaminylation’ pathway. An N-acetylmannosaminylation pathway is a biochemical pathway consisting of at least one of the enzymes and their respective genes chosen from the list comprising L-glutamine-D-fructose-6-phosphate aminotransferase, glucosamine-6-phosphate deaminase, phosphoglucosamine mutase, N-acetylglucosamine-6-phosphate deacetylase, glucosamine 6-phosphate N-acetyltransferase, N-acetylglucosamine-1-phosphate uridyltransferase, glucosamine-1-phosphate acetyltransferase, glucosamine-1-phosphate acetyltransferase, UDP-GlcNAc 2-epimerase and/or ManNAc kinase combined with a glycosyltransferase leading to a ManNAc-modified compound comprising a mono-, di- or oligosaccharide having an alpha or beta bound N-acetylmannosamine on said mono-, di- or oligosaccharide.

In a more preferred additional and/or alternative embodiment, the cell is metabolically engineered to comprise an N-acetylmannosaminylation pathway. In another even more preferred additional and/or alternative embodiment, the cell has been metabolically engineered to comprise an N-acetylmannosaminylation pathway wherein any one or more of the genes chosen from the list comprising L-glutamine-D-fructose-6-phosphate aminotransferase, glucosamine-6-phosphate deaminase, phosphoglucosamine mutase, N-acetylglucosamine-6-phosphate deacetylase, glucosamine 6-phosphate N-acetyltransferase, N-acetylglucosamine-1-phosphate uridyltransferase, glucosamine-1-phosphate acetyltransferase, glucosamine-1-phosphate acetyltransferase, UDP-GlcNAc 2-epimerase and/or ManNAc kinase and a glycosyltransferase transferring ManNAc has/have a modified and/or enhanced expression.

In another and/or additional preferred embodiment, the cell is metabolically engineered for an enhanced production of a saccharide, an enhanced uptake of one or more precursor(s) and/or acceptor(s) that is/are used in the synthesis of a saccharide, a better efflux of a saccharide, a decreased production of by-products like e.g. acids, an increased availability of co-factors like e.g. ATP, NADP, NADPH, and/or better metabolic flux through any one of the sialylation, fucosylation, galactosylation, N-acetylglucosaminylation, N-acetylgalactosaminylation, mannosylation, and/or N-acetylmannosaminylation pathway present in the cell.

In another preferred embodiment, the cell produces said saccharide from one or more internalized precursor(s) as defined herein. Preferably, said precursor is fed to the cell from the culture medium. In a more preferred embodiment, the cell synthesizes one or more precursor(s) that is/are involved in the production of said saccharide. In another preferred embodiment, the precursor(s) that is/are used by the cell for the production of said saccharide is/are completely converted into said saccharide. In another preferred embodiment, the precursor(s) that is/are used in said fermentation for the production of said saccharide is/are completely converted into said saccharide.

In another preferred embodiment, the cell is cultivated in culture medium comprising a carbon source comprising a monosaccharide, disaccharide, oligosaccharide, polysaccharide, polyol, glycerol, a complex medium including molasses, corn steep liquor, peptone, tryptone or yeast extract. Preferably, said carbon source is chosen from the list comprising glucose, N-acetylglucosamine (GlcNAc), glycerol, fructose, sucrose, maltose, lactose, arabinose, malto-oligosaccharides, maltotriose, sorbitol, xylose, rhamnose, galactose, mannose, methanol, ethanol, trehalose, starch, cellulose, hemi-cellulose, molasses, corn-steep liquor, high-fructose syrup, acetate, citrate, lactate and pyruvate. In a more preferred embodiment, the culture medium is a chemically defined medium. In an additional preferred embodiment, the culture medium is a minimal salt medium comprising sulphate, phosphate, chloride, ammonium, calcium, magnesium, sodium, potassium, iron, copper, zinc, manganese, cobalt, and/or selenium.

In another and/or additional preferred embodiment, the cell is cultivated in a chemically defined medium. In another and/or additional preferred embodiment, the cell is cultivated in a minimal salt medium comprising sulphate, phosphate, chloride, ammonium, calcium, magnesium, sodium, potassium, iron, copper, zinc, manganese, cobalt, and/or selenium.

In a specific embodiment of present invention, said fermentation broth comprising a saccharide and biomass is used in a method of the invention for the purification of said saccharide from said fermentation broth wherein said method comprises filtration of said fermentation broth on a membrane and subsequent diafiltration on said membrane under conditions permissive to produce or to collect a retentate comprising, consisting of or consisting essentially of said biomass and a permeate comprising said saccharide, specifically a permeate comprising essentially all or all of said saccharide, wherein said membrane comprises a molecular weight cut-off ranging from 0.3 to 5 kDa, a pore size ranging from 0.001 to 0.01 μm and/or a monovalent ion rejection ranging from 1 to 50%. A molecular weight cut-off ranging from 0.3 to 5 kDa is to be understood a molecular weight cut-off of 0.3 kDa, 0.4 kDa, 0.5 kDa, 0.6 kDa, 0.7 kDa, 0.8 kDa, 0.9 kDa, 1 kDa, 1.1 kDa, 1.2 kDa, 1.3 kDa, 1.4 kDa, 1.5 kDa, 1.6 kDa, 1.7 kDa, 1.8 kDa, 1.9 kDa, 2 kDa, 2.1 kDa, 2.2 kDa, 2.3 kDa, 2.4 kDa, 2.5 kDa, 2.6 kDa, 2.7 kDa, 2.8 kDa, 2.9 kDa, 3 kDa, 3.1 kDa, 3.2 kDa, 3.3 kDa, 3.4 kDa, 3.5 kDa, 3.6 kDa, 3.7 kDa, 3.8 kDa, 3.9 kDa, 4 kDa, 4.1 kDa, 4.2 kDa, 4.3 kDa, 4.4 kDa, 4.5 kDa, 4.6 kDa, 4.7 kDa, 4.8 kDa, 4.9 kDa or 5 kDa. In a preferred embodiment, the membrane comprises a molecular weight cut-off ranging from 0.5 to 4 kDa. A molecular weight cut-off ranging from 0.5 to 4 kDa is to be understood a molecular weight cut-off of 0.5 kDa, 0.6 kDa, 0.7 kDa, 0.8 kDa, 0.9 kDa, 1 kDa, 1.1 kDa, 1.2 kDa, 1.3 kDa, 1.4 kDa, 1.5 kDa, 1.6 kDa, 1.7 kDa, 1.8 kDa, 1.9 kDa, 2 kDa, 2.1 kDa, 2.2 kDa, 2.3 kDa, 2.4 kDa, 2.5 kDa, 2.6 kDa, 2.7 kDa, 2.8 kDa, 2.9 kDa, 3 kDa, 3.1 kDa, 3.2 kDa, 3.3 kDa, 3.4 kDa, 3.5 kDa, 3.6 kDa, 3.7 kDa, 3.8 kDa, 3.9 kDa or 4 kDa. In a more preferred embodiment, the membrane comprises a molecular weight cut-off ranging from 1 to 3.5 kDa. A molecular weight cut-off ranging from 1 to 3.5 kDa is to be understood a molecular weight cut-off of 1 kDa, 1.1 kDa, 1.2 kDa, 1.3 kDa, 1.4 kDa, 1.5 kDa, 1.6 kDa, 1.7 kDa, 1.8 kDa, 1.9 kDa, 2 kDa, 2.1 kDa, 2.2 kDa, 2.3 kDa, 2.4 kDa, 2.5 kDa, 2.6 kDa, 2.7 kDa, 2.8 kDa, 2.9 kDa, 3 kDa, 3.1 kDa, 3.2 kDa, 3.3 kDa, 3.4 kDa or 3.5 kDa. In an even more preferred embodiment, the membrane comprises a molecular weight cut-off ranging from 1.5 to 3 kDa. A molecular weight cut-off ranging from 1.5 to 3 kDa is to be understood a molecular weight cut-off of 1.5 kDa, 1.6 kDa, 1.7 kDa, 1.8 kDa, 1.9 kDa, 2 kDa, 2.1 kDa, 2.2 kDa, 2.3 kDa, 2.4 kDa, 2.5 kDa, 2.6 kDa, 2.7 kDa, 2.8 kDa, 2.9 kDa or 3 kDa. A pore size ranging from 0.001 to 0.01 μm is to be understood a pore size of 0.001 μm, 0.0015 μm, 0.002 μm, 0.0025 μm, 0.003 μm, 0.0035 μm, 0.004 μm, 0.0045 μm, 0.005 μm, 0.0055 μm, 0.006 μm, 0.0065 μm, 0.007 μm, 0.0075 μm, 0.008 μm, 0.0085 μm, 0.009 μm, 0.0095 μm, 0.0096 μm, 0.0097 μm, 0.0098 μm, 0.0099 am or 0.01 μm. A monovalent ion rejection ranging from 1 to 50% is to be understood a monovalent ion rejection of 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49% or 50%. In a preferred embodiment, the membrane comprises a monovalent ion rejection ranging from 5 to 40%. A monovalent ion rejection ranging from 5 to 40% is to be understood a monovalent ion rejection of 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39% or 40%. In a more preferred embodiment, the membrane comprises a monovalent ion rejection ranging from 10 to 20%. A monovalent ion rejection ranging from 10 to 20% is to be understood a monovalent ion rejection of 10%, 11%, 12%, 13%, 14%, 15%, 16%, 17%, 18%, 19% or 20%.

Examples of a monovalent ion comprise but are not limited to Na⁺, Cl⁻, Li⁺, K⁺, Cs⁺. In a preferred embodiment, the membrane comprises a rejection of Na⁺ of 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49% or 50%. In another and/or additional preferred embodiment, the membrane comprises a rejection of Cl⁻ of 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49% or 50%. In another and/or additional preferred embodiment, the membrane comprises a rejection of Li⁺ of 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49% or 50%. In another and/or additional preferred embodiment, the membrane comprises a rejection of K⁺ of 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49% or 50%. In another and/or additional preferred embodiment, the membrane comprises a rejection of Cs⁺ of 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49% or 50%.

In another and/or additional preferred embodiment, said membrane used in said filtration and subsequent diafiltration has a rejection of NaCl ranging from 1 to 40%. A rejection of NaCl ranging from 1 to 40% is to be understood a rejection of NaCl of 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39% or 40%. In a more preferred embodiment, said membrane used in said filtration and subsequent diafiltration has a rejection of NaCl ranging from 2 to 20%. A rejection of NaCl ranging from 2 to 20% is to be understood a rejection of NaCl of 2%, 3%, 4%, 5%, 6%, 7%, 8%, 9%, 10%, 11%, 12%, 13%, 14%, 15%, 16%, 17%, 18%, 19% or 20%. In an even more preferred embodiment, said membrane used in said filtration and subsequent diafiltration has a rejection of NaCl ranging from 5 to 10%. A rejection of NaCl ranging from 5 to 10% is to be understood a rejection of NaCl of 5%, 6%, 7%, 8%, 9% or 10%.

In another and/or additional preferred embodiment, said membrane comprises i) a molecular weight cut-off of 0.3 kDa, 0.4 kDa, 0.5 kDa, 0.6 kDa, 0.7 kDa, 0.8 kDa, 0.9 kDa, 1 kDa, 1.1 kDa, 1.2 kDa, 1.3 kDa, 1.4 kDa, 1.5 kDa, 1.6 kDa, 1.7 kDa, 1.8 kDa, 1.9 kDa, 2 kDa, 2.1 kDa, 2.2 kDa, 2.3 kDa, 2.4 kDa, 2.5 kDa, 2.6 kDa, 2.7 kDa, 2.8 kDa, 2.9 kDa, 3 kDa, 3.1 kDa, 3.2 kDa, 3.3 kDa, 3.4 kDa, 3.5 kDa, 3.6 kDa, 3.7 kDa, 3.8 kDa, 3.9 kDa, 4 kDa, 4.1 kDa, 4.2 kDa, 4.3 kDa, 4.4 kDa, 4.5 kDa, 4.6 kDa, 4.7 kDa, 4.8 kDa, 4.9 kDa or 5 kDa and ii) a rejection of NaCl of 10% or 20%.

In another and/or additional preferred embodiment, said membrane comprises i) a molecular weight cut-off of 0.3 kDa, 0.4 kDa, 0.5 kDa, 0.6 kDa, 0.7 kDa, 0.8 kDa, 0.9 kDa, 1 kDa, 1.1 kDa, 1.2 kDa, 1.3 kDa, 1.4 kDa, 1.5 kDa, 1.6 kDa, 1.7 kDa, 1.8 kDa, 1.9 kDa, 2 kDa, 2.1 kDa, 2.2 kDa, 2.3 kDa, 2.4 kDa, 2.5 kDa, 2.6 kDa, 2.7 kDa, 2.8 kDa, 2.9 kDa, 3 kDa, 3.1 kDa, 3.2 kDa, 3.3 kDa, 3.4 kDa, 3.5 kDa, 3.6 kDa, 3.7 kDa, 3.8 kDa, 3.9 kDa, 4 kDa, 4.1 kDa, 4.2 kDa, 4.3 kDa, 4.4 kDa, 4.5 kDa, 4.6 kDa, 4.7 kDa, 4.8 kDa, 4.9 kDa or 5 kDa and ii) a rejection of NaCl of 30% or 40%.

In another and/or additional preferred embodiment, said membrane comprises i) a molecular weight cut-off of 0.3 kDa, 0.4 kDa, 0.5 kDa, 0.6 kDa, 0.7 kDa, 0.8 kDa, 0.9 kDa, 1 kDa, 1.1 kDa, 1.2 kDa, 1.3 kDa, 1.4 kDa, 1.5 kDa, 1.6 kDa, 1.7 kDa, 1.8 kDa, 1.9 kDa, 2 kDa, 2.1 kDa, 2.2 kDa, 2.3 kDa, 2.4 kDa, 2.5 kDa, 2.6 kDa, 2.7 kDa, 2.8 kDa, 2.9 kDa, 3 kDa, 3.1 kDa, 3.2 kDa, 3.3 kDa, 3.4 kDa, 3.5 kDa, 3.6 kDa, 3.7 kDa, 3.8 kDa, 3.9 kDa, 4 kDa, 4.1 kDa, 4.2 kDa, 4.3 kDa, 4.4 kDa, 4.5 kDa, 4.6 kDa, 4.7 kDa, 4.8 kDa, 4.9 kDa or 5 kDa and ii) a rejection of NaCl of 7%.

In another and/or additional preferred embodiment, said membrane comprises i) a molecular weight cut-off of 0.3 kDa, 0.4 kDa, 0.5 kDa, 0.6 kDa, 0.7 kDa, 0.8 kDa, 0.9 kDa, 1 kDa, 1.1 kDa, 1.2 kDa, 1.3 kDa, 1.4 kDa, 1.5 kDa, 1.6 kDa, 1.7 kDa, 1.8 kDa, 1.9 kDa, 2 kDa, 2.1 kDa, 2.2 kDa, 2.3 kDa, 2.4 kDa, 2.5 kDa, 2.6 kDa, 2.7 kDa, 2.8 kDa, 2.9 kDa, 3 kDa, 3.1 kDa, 3.2 kDa, 3.3 kDa, 3.4 kDa, 3.5 kDa, 3.6 kDa, 3.7 kDa, 3.8 kDa, 3.9 kDa, 4 kDa, 4.1 kDa, 4.2 kDa, 4.3 kDa, 4.4 kDa, 4.5 kDa, 4.6 kDa, 4.7 kDa, 4.8 kDa, 4.9 kDa or 5 kDa, ii) a pore size of 0.001 μm, 0.0015 μm, 0.002 μm, 0.0025 μm, 0.003 μm, 0.0035 μm, 0.004 μm, 0.0045 μm, 0.005 μm, 0.0055 μm, 0.006 μm, 0.0065 μm, 0.007 μm, 0.0075 μm, 0.008 μm, 0.0085 μm, 0.009 μm, 0.0095 μm, 0.0096 μm, 0.0097 μm, 0.0098 μm, 0.0099 μm or 0.01 μm and iii) a rejection of NaCl of 10%.

In another and/or additional preferred embodiment, said membrane comprises i) a molecular weight cut-off of 0.3 kDa, 0.4 kDa, 0.5 kDa, 0.6 kDa, 0.7 kDa, 0.8 kDa, 0.9 kDa, 1 kDa, 1.1 kDa, 1.2 kDa, 1.3 kDa, 1.4 kDa, 1.5 kDa, 1.6 kDa, 1.7 kDa, 1.8 kDa, 1.9 kDa, 2 kDa, 2.1 kDa, 2.2 kDa, 2.3 kDa, 2.4 kDa, 2.5 kDa, 2.6 kDa, 2.7 kDa, 2.8 kDa, 2.9 kDa, 3 kDa, 3.1 kDa, 3.2 kDa, 3.3 kDa, 3.4 kDa, 3.5 kDa, 3.6 kDa, 3.7 kDa, 3.8 kDa, 3.9 kDa, 4 kDa, 4.1 kDa, 4.2 kDa, 4.3 kDa, 4.4 kDa, 4.5 kDa, 4.6 kDa, 4.7 kDa, 4.8 kDa, 4.9 kDa or 5 kDa, ii) a pore size of 0.001 μm, 0.0015 μm, 0.002 μm, 0.0025 μm, 0.003 μm, 0.0035 μm, 0.004 μm, 0.0045 μm, 0.005 μm, 0.0055 μm, 0.006 μm, 0.0065 μm, 0.007 μm, 0.0075 μm, 0.008 μm, 0.0085 μm, 0.009 μm, 0.0095 μm, 0.0096 μm, 0.0097 μm, 0.0098 μm, 0.0099 μm or 0.01 μm and iii) a rejection of NaCl of 20%. In another and/or additional preferred embodiment, said membrane comprises i) a molecular weight cut-off of 0.3 kDa, 0.4 kDa, 0.5 kDa, 0.6 kDa, 0.7 kDa, 0.8 kDa, 0.9 kDa, 1 kDa, 1.1 kDa, 1.2 kDa, 1.3 kDa, 1.4 kDa, 1.5 kDa, 1.6 kDa, 1.7 kDa, 1.8 kDa, 1.9 kDa, 2 kDa, 2.1 kDa, 2.2 kDa, 2.3 kDa, 2.4 kDa, 2.5 kDa, 2.6 kDa, 2.7 kDa, 2.8 kDa, 2.9 kDa, 3 kDa, 3.1 kDa, 3.2 kDa, 3.3 kDa, 3.4 kDa, 3.5 kDa, 3.6 kDa, 3.7 kDa, 3.8 kDa, 3.9 kDa, 4 kDa, 4.1 kDa, 4.2 kDa, 4.3 kDa, 4.4 kDa, 4.5 kDa, 4.6 kDa, 4.7 kDa, 4.8 kDa, 4.9 kDa or 5 kDa, ii) a pore size of 0.001 μm, 0.0015 μm, 0.002 μm, 0.0025 μm, 0.003 μm, 0.0035 μm, 0.004 μm, 0.0045 μm, 0.005 μm, 0.0055 μm, 0.006 μm, 0.0065 μm, 0.007 μm, 0.0075 μm, 0.008 μm, 0.0085 μm, 0.009 μm, 0.0095 μm, 0.0096 μm, 0.0097 μm, 0.0098 μm, 0.0099 μm or 0.01 μm and iii) a rejection of NaCl of 30%. In another and/or additional preferred embodiment, said membrane comprises i) a molecular weight cut-off of 0.3 kDa, 0.4 kDa, 0.5 kDa, 0.6 kDa, 0.7 kDa, 0.8 kDa, 0.9 kDa, 1 kDa, 1.1 kDa, 1.2 kDa, 1.3 kDa, 1.4 kDa, 1.5 kDa, 1.6 kDa, 1.7 kDa, 1.8 kDa, 1.9 kDa, 2 kDa, 2.1 kDa, 2.2 kDa, 2.3 kDa, 2.4 kDa, 2.5 kDa, 2.6 kDa, 2.7 kDa, 2.8 kDa, 2.9 kDa, 3 kDa, 3.1 kDa, 3.2 kDa, 3.3 kDa, 3.4 kDa, 3.5 kDa, 3.6 kDa, 3.7 kDa, 3.8 kDa, 3.9 kDa, 4 kDa, 4.1 kDa, 4.2 kDa, 4.3 kDa, 4.4 kDa, 4.5 kDa, 4.6 kDa, 4.7 kDa, 4.8 kDa, 4.9 kDa or 5 kDa, ii) a pore size of 0.001 μm, 0.0015 μm, 0.002 μm, 0.0025 μm, 0.003 μm, 0.0035 μm, 0.004 μm, 0.0045 μm, 0.005 μm, 0.0055 μm, 0.006 μm, 0.0065 μm, 0.007 μm, 0.0075 μm, 0.008 μm, 0.0085 μm, 0.009 μm, 0.0095 μm, 0.0096 μm, 0.0097 μm, 0.0098 μm, 0.0099 μm or 0.01 μm and iii) a rejection of NaCl of 40%.

In another and/or additional preferred embodiment, said membrane comprises i) a molecular weight cut-off of 0.3 kDa, 0.4 kDa, 0.5 kDa, 0.6 kDa, 0.7 kDa, 0.8 kDa, 0.9 kDa, 1 kDa, 1.1 kDa, 1.2 kDa, 1.3 kDa, 1.4 kDa, 1.5 kDa, 1.6 kDa, 1.7 kDa, 1.8 kDa, 1.9 kDa, 2 kDa, 2.1 kDa, 2.2 kDa, 2.3 kDa, 2.4 kDa, 2.5 kDa, 2.6 kDa, 2.7 kDa, 2.8 kDa, 2.9 kDa, 3 kDa, 3.1 kDa, 3.2 kDa, 3.3 kDa, 3.4 kDa, 3.5 kDa, 3.6 kDa, 3.7 kDa, 3.8 kDa, 3.9 kDa, 4 kDa, 4.1 kDa, 4.2 kDa, 4.3 kDa, 4.4 kDa, 4.5 kDa, 4.6 kDa, 4.7 kDa, 4.8 kDa, 4.9 kDa or 5 kDa, ii) a pore size of 0.001 μm, 0.0015 μm, 0.002 μm, 0.0025 μm, 0.003 μm, 0.0035 μm, 0.004 μm, 0.0045 μm, 0.005 μm, 0.0055 μm, 0.006 μm, 0.0065 μm, 0.007 μm, 0.0075 μm, 0.008 μm, 0.0085 μm, 0.009 μm, 0.0095 μm, 0.0096 μm, 0.0097 μm, 0.0098 μm, 0.0099 μm or 0.01 μm and iii) a rejection of NaCl of 7%.

In another and/or additional preferred embodiment, said membrane comprises a divalent ion rejection. Examples of a divalent ion comprise but are not limited to Mg²⁺, Ca²⁺, Ba²⁺, Fe²⁺, Cu²⁺, Mn²⁺, Cd²⁺, Zn²⁺.

In another and/or additional preferred embodiment, said membrane used in said filtration and subsequent diafiltration has a rejection of MgSO₄ ranging from 10 to 98%. A rejection of MgSO₄ ranging from 10 to 98% is to be understood a rejection of MgSO₄ of 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97% or 98%. In a more preferred embodiment, said membrane used in said filtration and subsequent diafiltration has a rejection of MgSO₄ ranging from 20 to 97%. A rejection of MgSO₄ ranging from 20 to 97% is to be understood a rejection of MgSO₄ of 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96% or 97%. In an even more preferred embodiment, said membrane used in said filtration and subsequent diafiltration has a rejection of MgSO₄ ranging from 50 to 95%. A rejection of MgSO₄ ranging from 50 to 95% is to be understood a rejection of MgSO₄ of 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94% or 95%. In an even more preferred embodiment, said membrane used in said filtration and subsequent diafiltration has a rejection of MgSO₄ ranging from 70 to 94%. A rejection of MgSO₄ ranging from 70 to 94% is to be understood a rejection of MgSO₄ of 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93% or 94%. In an even more preferred embodiment, said membrane used in said filtration and subsequent diafiltration has a rejection of MgSO₄ ranging from 70 to 90%. A rejection of MgSO₄ ranging from 70 to 90% is to be understood a rejection of MgSO₄ of 70%, 71%, 72%, 73%, 74%, 75%, 76%, 77%, 78%, 79%, 80%, 81%, 82%, 83%, 84%, 85%, 86%, 87%, 88%, 89% or 90%.

In another and/or additional preferred embodiment, said membrane comprises i) a molecular weight cut-off of 1 kDa, 1.1 kDa, 1.2 kDa, 1.3 kDa, 1.4 kDa, 1.5 kDa, 1.6 kDa, 1.7 kDa, 1.8 kDa, 1.9 kDa, 2 kDa, 2.1 kDa, 2.2 kDa, 2.3 kDa, 2.4 kDa, 2.5 kDa, 2.6 kDa, 2.7 kDa, 2.8 kDa, 2.9 kDa, 3 kDa, 3.1 kDa, 3.2 kDa, 3.3 kDa, 3.4 kDa, 3.5 kDa or 3.6 kDa, ii) a rejection of NaCl of 10% and iii) a rejection of MgSO₄ of 70%. In another and/or additional preferred embodiment, said membrane comprises i) a molecular weight cut-off of 1 kDa, 1.1 kDa, 1.2 kDa, 1.3 kDa, 1.4 kDa, 1.5 kDa, 1.6 kDa, 1.7 kDa, 1.8 kDa, 1.9 kDa, 2 kDa, 2.1 kDa, 2.2 kDa, 2.3 kDa, 2.4 kDa, 2.5 kDa, 2.6 kDa, 2.7 kDa, 2.8 kDa, 2.9 kDa, 3 kDa, 3.1 kDa, 3.2 kDa, 3.3 kDa, 3.4 kDa, 3.5 kDa or 3.6 kDa, ii) a pore size of 0.001 μm, 0.0015 μm, 0.002 μm, 0.0025 μm, 0.003 μm, 0.0035 μm, 0.004 μm, 0.0045 μm, 0.005 μm, 0.0055 μm, 0.006 μm, 0.0065 μm, 0.007 μm, 0.0075 μm, 0.008 μm, 0.0085 μm, 0.009 μm, 0.0095 μm, 0.0096 μm, 0.0097 μm, 0.0098 μm, 0.0099 μm or 0.01 μm, iii) a rejection of NaCl of 10% and iv) a rejection of MgSO₄ of 70%. In another and/or additional preferred embodiment, said membrane comprises i) a molecular weight cut-off of 1 kDa, 1.1 kDa, 1.2 kDa, 1.3 kDa, 1.4 kDa, 1.5 kDa, 1.6 kDa, 1.7 kDa, 1.8 kDa, 1.9 kDa, 2 kDa, 2.1 kDa, 2.2 kDa, 2.3 kDa, 2.4 kDa, 2.5 kDa, 2.6 kDa, 2.7 kDa, 2.8 kDa, 2.9 kDa, 3 kDa, 3.1 kDa, 3.2 kDa, 3.3 kDa, 3.4 kDa, 3.5 kDa or 3.6 kDa, ii) a rejection of NaCl of 10% and iii) a rejection of MgSO₄ of 75%. In another and/or additional preferred embodiment, said membrane comprises i) a molecular weight cut-off of 1 kDa, 1.1 kDa, 1.2 kDa, 1.3 kDa, 1.4 kDa, 1.5 kDa, 1.6 kDa, 1.7 kDa, 1.8 kDa, 1.9 kDa, 2 kDa, 2.1 kDa, 2.2 kDa, 2.3 kDa, 2.4 kDa, 2.5 kDa, 2.6 kDa, 2.7 kDa, 2.8 kDa, 2.9 kDa, 3 kDa, 3.1 kDa, 3.2 kDa, 3.3 kDa, 3.4 kDa, 3.5 kDa or 3.6 kDa, ii) a pore size of 0.001 μm, 0.0015 μm, 0.002 μm, 0.0025 μm, 0.003 μm, 0.0035 μm, 0.004 μm, 0.0045 μm, 0.005 μm, 0.0055 μm, 0.006 μm, 0.0065 μm, 0.007 μm, 0.0075 μm, 0.008 μm, 0.0085 μm, 0.009 μm, 0.0095 μm, 0.0096 μm, 0.0097 μm, 0.0098 μm, 0.0099 am or 0.01 μm, iii) a rejection of NaCl of 10% and iv) a rejection of MgSO₄ of 75%. In another and/or additional preferred embodiment, said membrane comprises i) a molecular weight cut-off of 1 kDa, 1.1 kDa, 1.2 kDa, 1.3 kDa, 1.4 kDa, 1.5 kDa, 1.6 kDa, 1.7 kDa, 1.8 kDa, 1.9 kDa, 2 kDa, 2.1 kDa, 2.2 kDa, 2.3 kDa, 2.4 kDa, 2.5 kDa, 2.6 kDa, 2.7 kDa, 2.8 kDa, 2.9 kDa, 3 kDa, 3.1 kDa, 3.2 kDa, 3.3 kDa, 3.4 kDa, 3.5 kDa or 3.6 kDa, ii) a rejection of NaCl of 10% and iii) a rejection of MgSO₄ of 80%. In another and/or additional preferred embodiment, said membrane comprises i) a molecular weight cut-off of 1 kDa, 1.1 kDa, 1.2 kDa, 1.3 kDa, 1.4 kDa, 1.5 kDa, 1.6 kDa, 1.7 kDa, 1.8 kDa, 1.9 kDa, 2 kDa, 2.1 kDa, 2.2 kDa, 2.3 kDa, 2.4 kDa, 2.5 kDa, 2.6 kDa, 2.7 kDa, 2.8 kDa, 2.9 kDa, 3 kDa, 3.1 kDa, 3.2 kDa, 3.3 kDa, 3.4 kDa, 3.5 kDa or 3.6 kDa, ii) a pore size of 0.001 μm, 0.0015 μm, 0.002 μm, 0.0025 μm, 0.003 μm, 0.0035 μm, 0.004 μm, 0.0045 μm, 0.005 μm, 0.0055 μm, 0.006 μm, 0.0065 μm, 0.007 μm, 0.0075 μm, 0.008 μm, 0.0085 μm, 0.009 μm, 0.0095 μm, 0.0096 μm, 0.0097 μm, 0.0098 μm, 0.0099 μm or 0.01 μm, iii) a rejection of NaCl of 10% and iv) a rejection of MgSO₄ of 80%. In another and/or additional preferred embodiment, said membrane comprises i) a molecular weight cut-off of 1 kDa, 1.1 kDa, 1.2 kDa, 1.3 kDa, 1.4 kDa, 1.5 kDa, 1.6 kDa, 1.7 kDa, 1.8 kDa, 1.9 kDa, 2 kDa, 2.1 kDa, 2.2 kDa, 2.3 kDa, 2.4 kDa, 2.5 kDa, 2.6 kDa, 2.7 kDa, 2.8 kDa, 2.9 kDa, 3 kDa, 3.1 kDa, 3.2 kDa, 3.3 kDa, 3.4 kDa, 3.5 kDa or 3.6 kDa, ii) a rejection of NaCl of 10% and iii) a rejection of MgSO₄ of 85%. In another and/or additional preferred embodiment, said membrane comprises i) a molecular weight cut-off of 1 kDa, 1.1 kDa, 1.2 kDa, 1.3 kDa, 1.4 kDa, 1.5 kDa, 1.6 kDa, 1.7 kDa, 1.8 kDa, 1.9 kDa, 2 kDa, 2.1 kDa, 2.2 kDa, 2.3 kDa, 2.4 kDa, 2.5 kDa, 2.6 kDa, 2.7 kDa, 2.8 kDa, 2.9 kDa, 3 kDa, 3.1 kDa, 3.2 kDa, 3.3 kDa, 3.4 kDa, 3.5 kDa or 3.6 kDa, ii) a pore size of 0.001 μm, 0.0015 μm, 0.002 μm, 0.0025 μm, 0.003 μm, 0.0035 μm, 0.004 μm, 0.0045 μm, 0.005 μm, 0.0055 μm, 0.006 μm, 0.0065 μm, 0.007 μm, 0.0075 μm, 0.008 μm, 0.0085 μm, 0.009 μm, 0.0095 μm, 0.0096 μm, 0.0097 μm, 0.0098 μm, 0.0099 am or 0.01 μm, iii) a rejection of NaCl of 10% and iv) a rejection of MgSO₄ of 85%. In another and/or additional preferred embodiment, said membrane comprises i) a molecular weight cut-off of 1 kDa, 1.1 kDa, 1.2 kDa, 1.3 kDa, 1.4 kDa, 1.5 kDa, 1.6 kDa, 1.7 kDa, 1.8 kDa, 1.9 kDa, 2 kDa, 2.1 kDa, 2.2 kDa, 2.3 kDa, 2.4 kDa, 2.5 kDa, 2.6 kDa, 2.7 kDa, 2.8 kDa, 2.9 kDa, 3 kDa, 3.1 kDa, 3.2 kDa, 3.3 kDa, 3.4 kDa, 3.5 kDa or 3.6 kDa, ii) a rejection of NaCl of 10% and iii) a rejection of MgSO₄ of 90%. In another and/or additional preferred embodiment, said membrane comprises i) a molecular weight cut-off of 1 kDa, 1.1 kDa, 1.2 kDa, 1.3 kDa, 1.4 kDa, 1.5 kDa, 1.6 kDa, 1.7 kDa, 1.8 kDa, 1.9 kDa, 2 kDa, 2.1 kDa, 2.2 kDa, 2.3 kDa, 2.4 kDa, 2.5 kDa, 2.6 kDa, 2.7 kDa, 2.8 kDa, 2.9 kDa, 3 kDa, 3.1 kDa, 3.2 kDa, 3.3 kDa, 3.4 kDa, 3.5 kDa or 3.6 kDa, ii) a pore size of 0.001 μm, 0.0015 μm, 0.002 μm, 0.0025 μm, 0.003 μm, 0.0035 μm, 0.004 μm, 0.0045 μm, 0.005 μm, 0.0055 μm, 0.006 μm, 0.0065 μm, 0.007 μm, 0.0075 μm, 0.008 μm, 0.0085 μm, 0.009 μm, 0.0095 μm, 0.0096 μm, 0.0097 μm, 0.0098 μm, 0.0099 μm or 0.01 μm, iii) a rejection of NaCl of 10% and iv) a rejection of MgSO₄ of 90%.

In another and/or additional preferred embodiment, said membrane comprises i) a molecular weight cut-off of 1 kDa, 1.1 kDa, 1.2 kDa, 1.3 kDa, 1.4 kDa, 1.5 kDa, 1.6 kDa, 1.7 kDa, 1.8 kDa, 1.9 kDa, 2 kDa, 2.1 kDa, 2.2 kDa, 2.3 kDa, 2.4 kDa, 2.5 kDa, 2.6 kDa, 2.7 kDa, 2.8 kDa, 2.9 kDa, 3 kDa, 3.1 kDa, 3.2 kDa, 3.3 kDa, 3.4 kDa, 3.5 kDa or 3.6 kDa, ii) a rejection of NaCl of 20% and iii) a rejection of MgSO₄ of 70%. In another and/or additional preferred embodiment, said membrane comprises i) a molecular weight cut-off of 1 kDa, 1.1 kDa, 1.2 kDa, 1.3 kDa, 1.4 kDa, 1.5 kDa, 1.6 kDa, 1.7 kDa, 1.8 kDa, 1.9 kDa, 2 kDa, 2.1 kDa, 2.2 kDa, 2.3 kDa, 2.4 kDa, 2.5 kDa, 2.6 kDa, 2.7 kDa, 2.8 kDa, 2.9 kDa, 3 kDa, 3.1 kDa, 3.2 kDa, 3.3 kDa, 3.4 kDa, 3.5 kDa or 3.6 kDa, ii) a pore size of 0.001 μm, 0.0015 μm, 0.002 μm, 0.0025 μm, 0.003 μm, 0.0035 μm, 0.004 μm, 0.0045 μm, 0.005 μm, 0.0055 μm, 0.006 μm, 0.0065 μm, 0.007 μm, 0.0075 μm, 0.008 μm, 0.0085 μm, 0.009 μm, 0.0095 μm, 0.0096 μm, 0.0097 μm, 0.0098 μm, 0.0099 μm or 0.01 μm, iii) a rejection of NaCl of 20% and iv) a rejection of MgSO₄ of 70%. In another and/or additional preferred embodiment, said membrane comprises i) a molecular weight cut-off of 1 kDa, 1.1 kDa, 1.2 kDa, 1.3 kDa, 1.4 kDa, 1.5 kDa, 1.6 kDa, 1.7 kDa, 1.8 kDa, 1.9 kDa, 2 kDa, 2.1 kDa, 2.2 kDa, 2.3 kDa, 2.4 kDa, 2.5 kDa, 2.6 kDa, 2.7 kDa, 2.8 kDa, 2.9 kDa, 3 kDa, 3.1 kDa, 3.2 kDa, 3.3 kDa, 3.4 kDa, 3.5 kDa or 3.6 kDa, ii) a rejection of NaCl of 20% and iii) a rejection of MgSO₄ of 75%. In another and/or additional preferred embodiment, said membrane comprises i) a molecular weight cut-off of 1 kDa, 1.1 kDa, 1.2 kDa, 1.3 kDa, 1.4 kDa, 1.5 kDa, 1.6 kDa, 1.7 kDa, 1.8 kDa, 1.9 kDa, 2 kDa, 2.1 kDa, 2.2 kDa, 2.3 kDa, 2.4 kDa, 2.5 kDa, 2.6 kDa, 2.7 kDa, 2.8 kDa, 2.9 kDa, 3 kDa, 3.1 kDa, 3.2 kDa, 3.3 kDa, 3.4 kDa, 3.5 kDa or 3.6 kDa, ii) a pore size of 0.001 μm, 0.0015 μm, 0.002 μm, 0.0025 μm, 0.003 μm, 0.0035 μm, 0.004 μm, 0.0045 μm, 0.005 μm, 0.0055 μm, 0.006 μm, 0.0065 μm, 0.007 μm, 0.0075 μm, 0.008 μm, 0.0085 μm, 0.009 μm, 0.0095 μm, 0.0096 μm, 0.0097 μm, 0.0098 μm, 0.0099 am or 0.01 μm, iii) a rejection of NaCl of 20% and iv) a rejection of MgSO₄ of 75%. In another and/or additional preferred embodiment, said membrane comprises i) a molecular weight cut-off of 1 kDa, 1.1 kDa, 1.2 kDa, 1.3 kDa, 1.4 kDa, 1.5 kDa, 1.6 kDa, 1.7 kDa, 1.8 kDa, 1.9 kDa, 2 kDa, 2.1 kDa, 2.2 kDa, 2.3 kDa, 2.4 kDa, 2.5 kDa, 2.6 kDa, 2.7 kDa, 2.8 kDa, 2.9 kDa, 3 kDa, 3.1 kDa, 3.2 kDa, 3.3 kDa, 3.4 kDa, 3.5 kDa or 3.6 kDa, ii) a rejection of NaCl of 20% and iii) a rejection of MgSO₄ of 80%. In another and/or additional preferred embodiment, said membrane comprises i) a molecular weight cut-off of 1 kDa, 1.1 kDa, 1.2 kDa, 1.3 kDa, 1.4 kDa, 1.5 kDa, 1.6 kDa, 1.7 kDa, 1.8 kDa, 1.9 kDa, 2 kDa, 2.1 kDa, 2.2 kDa, 2.3 kDa, 2.4 kDa, 2.5 kDa, 2.6 kDa, 2.7 kDa, 2.8 kDa, 2.9 kDa, 3 kDa, 3.1 kDa, 3.2 kDa, 3.3 kDa, 3.4 kDa, 3.5 kDa or 3.6 kDa, ii) a pore size of 0.001 μm, 0.0015 μm, 0.002 μm, 0.0025 μm, 0.003 μm, 0.0035 μm, 0.004 μm, 0.0045 μm, 0.005 μm, 0.0055 μm, 0.006 μm, 0.0065 μm, 0.007 μm, 0.0075 μm, 0.008 μm, 0.0085 μm, 0.009 μm, 0.0095 μm, 0.0096 μm, 0.0097 μm, 0.0098 μm, 0.0099 μm or 0.01 μm, iii) a rejection of NaCl of 20% and iv) a rejection of MgSO₄ of 80%. In another and/or additional preferred embodiment, said membrane comprises i) a molecular weight cut-off of 1 kDa, 1.1 kDa, 1.2 kDa, 1.3 kDa, 1.4 kDa, 1.5 kDa, 1.6 kDa, 1.7 kDa, 1.8 kDa, 1.9 kDa, 2 kDa, 2.1 kDa, 2.2 kDa, 2.3 kDa, 2.4 kDa, 2.5 kDa, 2.6 kDa, 2.7 kDa, 2.8 kDa, 2.9 kDa, 3 kDa, 3.1 kDa, 3.2 kDa, 3.3 kDa, 3.4 kDa, 3.5 kDa or 3.6 kDa, ii) a rejection of NaCl of 20% and iii) a rejection of MgSO₄ of 85%. In another and/or additional preferred embodiment, said membrane comprises i) a molecular weight cut-off of 1 kDa, 1.1 kDa, 1.2 kDa, 1.3 kDa, 1.4 kDa, 1.5 kDa, 1.6 kDa, 1.7 kDa, 1.8 kDa, 1.9 kDa, 2 kDa, 2.1 kDa, 2.2 kDa, 2.3 kDa, 2.4 kDa, 2.5 kDa, 2.6 kDa, 2.7 kDa, 2.8 kDa, 2.9 kDa, 3 kDa, 3.1 kDa, 3.2 kDa, 3.3 kDa, 3.4 kDa, 3.5 kDa or 3.6 kDa, ii) a pore size of 0.001 μm, 0.0015 μm, 0.002 μm, 0.0025 μm, 0.003 μm, 0.0035 μm, 0.004 μm, 0.0045 μm, 0.005 μm, 0.0055 μm, 0.006 μm, 0.0065 μm, 0.007 μm, 0.0075 μm, 0.008 μm, 0.0085 μm, 0.009 μm, 0.0095 μm, 0.0096 μm, 0.0097 μm, 0.0098 μm, 0.0099 am or 0.01 μm, iii) a rejection of NaCl of 20% and iv) a rejection of MgSO₄ of 85%. In another and/or additional preferred embodiment, said membrane comprises i) a molecular weight cut-off of 1 kDa, 1.1 kDa, 1.2 kDa, 1.3 kDa, 1.4 kDa, 1.5 kDa, 1.6 kDa, 1.7 kDa, 1.8 kDa, 1.9 kDa, 2 kDa, 2.1 kDa, 2.2 kDa, 2.3 kDa, 2.4 kDa, 2.5 kDa, 2.6 kDa, 2.7 kDa, 2.8 kDa, 2.9 kDa, 3 kDa, 3.1 kDa, 3.2 kDa, 3.3 kDa, 3.4 kDa, 3.5 kDa or 3.6 kDa, ii) a rejection of NaCl of 20% and iii) a rejection of MgSO₄ of 90%. In another and/or additional preferred embodiment, said membrane comprises i) a molecular weight cut-off of 1 kDa, 1.1 kDa, 1.2 kDa, 1.3 kDa, 1.4 kDa, 1.5 kDa, 1.6 kDa, 1.7 kDa, 1.8 kDa, 1.9 kDa, 2 kDa, 2.1 kDa, 2.2 kDa, 2.3 kDa, 2.4 kDa, 2.5 kDa, 2.6 kDa, 2.7 kDa, 2.8 kDa, 2.9 kDa, 3 kDa, 3.1 kDa, 3.2 kDa, 3.3 kDa, 3.4 kDa, 3.5 kDa or 3.6 kDa, ii) a pore size of 0.001 μm, 0.0015 μm, 0.002 μm, 0.0025 μm, 0.003 μm, 0.0035 μm, 0.004 μm, 0.0045 μm, 0.005 μm, 0.0055 μm, 0.006 μm, 0.0065 μm, 0.007 μm, 0.0075 μm, 0.008 μm, 0.0085 μm, 0.009 μm, 0.0095 μm, 0.0096 μm, 0.0097 μm, 0.0098 μm, 0.0099 μm or 0.01 μm, iii) a rejection of NaCl of 20% and iv) a rejection of MgSO₄ of 90%.

In another and/or additional preferred embodiment, said membrane comprises i) a molecular weight cut-off of 1 kDa, 1.1 kDa, 1.2 kDa, 1.3 kDa, 1.4 kDa, 1.5 kDa, 1.6 kDa, 1.7 kDa, 1.8 kDa, 1.9 kDa, 2 kDa, 2.1 kDa, 2.2 kDa, 2.3 kDa, 2.4 kDa, 2.5 kDa, 2.6 kDa, 2.7 kDa, 2.8 kDa, 2.9 kDa, 3 kDa, 3.1 kDa, 3.2 kDa, 3.3 kDa, 3.4 kDa, 3.5 kDa or 3.6 kDa, ii) a rejection of NaCl of 40% and iii) a rejection of MgSO₄ of 70%. In another and/or additional preferred embodiment, said membrane comprises i) a molecular weight cut-off of 1 kDa, 1.1 kDa, 1.2 kDa, 1.3 kDa, 1.4 kDa, 1.5 kDa, 1.6 kDa, 1.7 kDa, 1.8 kDa, 1.9 kDa, 2 kDa, 2.1 kDa, 2.2 kDa, 2.3 kDa, 2.4 kDa, 2.5 kDa, 2.6 kDa, 2.7 kDa, 2.8 kDa, 2.9 kDa, 3 kDa, 3.1 kDa, 3.2 kDa, 3.3 kDa, 3.4 kDa, 3.5 kDa or 3.6 kDa, ii) a pore size of 0.001 μm, 0.0015 μm, 0.002 μm, 0.0025 μm, 0.003 μm, 0.0035 μm, 0.004 μm, 0.0045 μm, 0.005 μm, 0.0055 μm, 0.006 μm, 0.0065 μm, 0.007 μm, 0.0075 μm, 0.008 μm, 0.0085 μm, 0.009 μm, 0.0095 μm, 0.0096 μm, 0.0097 μm, 0.0098 μm, 0.0099 μm or 0.01 μm, iii) a rejection of NaCl of 40% and iv) a rejection of MgSO₄ of 70%. In another and/or additional preferred embodiment, said membrane comprises i) a molecular weight cut-off of 1 kDa, 1.1 kDa, 1.2 kDa, 1.3 kDa, 1.4 kDa, 1.5 kDa, 1.6 kDa, 1.7 kDa, 1.8 kDa, 1.9 kDa, 2 kDa, 2.1 kDa, 2.2 kDa, 2.3 kDa, 2.4 kDa, 2.5 kDa, 2.6 kDa, 2.7 kDa, 2.8 kDa, 2.9 kDa, 3 kDa, 3.1 kDa, 3.2 kDa, 3.3 kDa, 3.4 kDa, 3.5 kDa or 3.6 kDa, ii) a rejection of NaCl of 40% and iii) a rejection of MgSO₄ of 75%. In another and/or additional preferred embodiment, said membrane comprises i) a molecular weight cut-off of 1 kDa, 1.1 kDa, 1.2 kDa, 1.3 kDa, 1.4 kDa, 1.5 kDa, 1.6 kDa, 1.7 kDa, 1.8 kDa, 1.9 kDa, 2 kDa, 2.1 kDa, 2.2 kDa, 2.3 kDa, 2.4 kDa, 2.5 kDa, 2.6 kDa, 2.7 kDa, 2.8 kDa, 2.9 kDa, 3 kDa, 3.1 kDa, 3.2 kDa, 3.3 kDa, 3.4 kDa, 3.5 kDa or 3.6 kDa, ii) a pore size of 0.001 μm, 0.0015 μm, 0.002 μm, 0.0025 μm, 0.003 μm, 0.0035 μm, 0.004 μm, 0.0045 μm, 0.005 μm, 0.0055 μm, 0.006 μm, 0.0065 μm, 0.007 μm, 0.0075 μm, 0.008 μm, 0.0085 μm, 0.009 μm, 0.0095 μm, 0.0096 μm, 0.0097 μm, 0.0098 μm, 0.0099 am or 0.01 μm, iii) a rejection of NaCl of 40% and iv) a rejection of MgSO₄ of 75%. In another and/or additional preferred embodiment, said membrane comprises i) a molecular weight cut-off of 1 kDa, 1.1 kDa, 1.2 kDa, 1.3 kDa, 1.4 kDa, 1.5 kDa, 1.6 kDa, 1.7 kDa, 1.8 kDa, 1.9 kDa, 2 kDa, 2.1 kDa, 2.2 kDa, 2.3 kDa, 2.4 kDa, 2.5 kDa, 2.6 kDa, 2.7 kDa, 2.8 kDa, 2.9 kDa, 3 kDa, 3.1 kDa, 3.2 kDa, 3.3 kDa, 3.4 kDa, 3.5 kDa or 3.6 kDa, ii) a rejection of NaCl of 40% and iii) a rejection of MgSO₄ of 80%. In another and/or additional preferred embodiment, said membrane comprises i) a molecular weight cut-off of 1 kDa, 1.1 kDa, 1.2 kDa, 1.3 kDa, 1.4 kDa, 1.5 kDa, 1.6 kDa, 1.7 kDa, 1.8 kDa, 1.9 kDa, 2 kDa, 2.1 kDa, 2.2 kDa, 2.3 kDa, 2.4 kDa, 2.5 kDa, 2.6 kDa, 2.7 kDa, 2.8 kDa, 2.9 kDa, 3 kDa, 3.1 kDa, 3.2 kDa, 3.3 kDa, 3.4 kDa, 3.5 kDa or 3.6 kDa, ii) a pore size of 0.001 μm, 0.0015 μm, 0.002 μm, 0.0025 μm, 0.003 μm, 0.0035 μm, 0.004 μm, 0.0045 μm, 0.005 μm, 0.0055 μm, 0.006 μm, 0.0065 μm, 0.007 μm, 0.0075 μm, 0.008 μm, 0.0085 μm, 0.009 μm, 0.0095 μm, 0.0096 μm, 0.0097 μm, 0.0098 μm, 0.0099 μm or 0.01 μm, iii) a rejection of NaCl of 40% and iv) a rejection of MgSO₄ of 80%. In another and/or additional preferred embodiment, said membrane comprises i) a molecular weight cut-off of 1 kDa, 1.1 kDa, 1.2 kDa, 1.3 kDa, 1.4 kDa, 1.5 kDa, 1.6 kDa, 1.7 kDa, 1.8 kDa, 1.9 kDa, 2 kDa, 2.1 kDa, 2.2 kDa, 2.3 kDa, 2.4 kDa, 2.5 kDa, 2.6 kDa, 2.7 kDa, 2.8 kDa, 2.9 kDa, 3 kDa, 3.1 kDa, 3.2 kDa, 3.3 kDa, 3.4 kDa, 3.5 kDa or 3.6 kDa, ii) a rejection of NaCl of 40% and iii) a rejection of MgSO₄ of 85%. In another and/or additional preferred embodiment, said membrane comprises i) a molecular weight cut-off of 1 kDa, 1.1 kDa, 1.2 kDa, 1.3 kDa, 1.4 kDa, 1.5 kDa, 1.6 kDa, 1.7 kDa, 1.8 kDa, 1.9 kDa, 2 kDa, 2.1 kDa, 2.2 kDa, 2.3 kDa, 2.4 kDa, 2.5 kDa, 2.6 kDa, 2.7 kDa, 2.8 kDa, 2.9 kDa, 3 kDa, 3.1 kDa, 3.2 kDa, 3.3 kDa, 3.4 kDa, 3.5 kDa or 3.6 kDa, ii) a pore size of 0.001 μm, 0.0015 μm, 0.002 μm, 0.0025 μm, 0.003 μm, 0.0035 μm, 0.004 μm, 0.0045 μm, 0.005 μm, 0.0055 μm, 0.006 μm, 0.0065 μm, 0.007 μm, 0.0075 μm, 0.008 μm, 0.0085 μm, 0.009 μm, 0.0095 μm, 0.0096 μm, 0.0097 μm, 0.0098 μm, 0.0099 am or 0.01 μm, iii) a rejection of NaCl of 40% and iv) a rejection of MgSO₄ of 85%. In another and/or additional preferred embodiment, said membrane comprises i) a molecular weight cut-off of 1 kDa, 1.1 kDa, 1.2 kDa, 1.3 kDa, 1.4 kDa, 1.5 kDa, 1.6 kDa, 1.7 kDa, 1.8 kDa, 1.9 kDa, 2 kDa, 2.1 kDa, 2.2 kDa, 2.3 kDa, 2.4 kDa, 2.5 kDa, 2.6 kDa, 2.7 kDa, 2.8 kDa, 2.9 kDa, 3 kDa, 3.1 kDa, 3.2 kDa, 3.3 kDa, 3.4 kDa, 3.5 kDa or 3.6 kDa, ii) a rejection of NaCl of 40% and iii) a rejection of MgSO₄ of 90%. In another and/or additional preferred embodiment, said membrane comprises i) a molecular weight cut-off of 1 kDa, 1.1 kDa, 1.2 kDa, 1.3 kDa, 1.4 kDa, 1.5 kDa, 1.6 kDa, 1.7 kDa, 1.8 kDa, 1.9 kDa, 2 kDa, 2.1 kDa, 2.2 kDa, 2.3 kDa, 2.4 kDa, 2.5 kDa, 2.6 kDa, 2.7 kDa, 2.8 kDa, 2.9 kDa, 3 kDa, 3.1 kDa, 3.2 kDa, 3.3 kDa, 3.4 kDa, 3.5 kDa or 3.6 kDa, ii) a pore size of 0.001 μm, 0.0015 μm, 0.002 μm, 0.0025 μm, 0.003 μm, 0.0035 μm, 0.004 μm, 0.0045 μm, 0.005 μm, 0.0055 μm, 0.006 μm, 0.0065 μm, 0.007 μm, 0.0075 μm, 0.008 μm, 0.0085 μm, 0.009 μm, 0.0095 μm, 0.0096 μm, 0.0097 μm, 0.0098 μm, 0.0099 μm or 0.01 μm, iii) a rejection of NaCl of 40% and iv) a rejection of MgSO₄ of 90%.

In another and/or additional preferred embodiment, said membrane comprises i) a molecular weight cut-off of 0.3 kDa, 0.4 kDa or 0.5 kDa, ii) a rejection of NaCl of 20% and iii) a rejection of MgSO₄ of 94%. In another and/or additional preferred embodiment, said membrane comprises i) a molecular weight cut-off of 0.3 kDa, 0.4 kDa or 0.5 kDa, ii) a rejection of NaCl of 20% and iii) a rejection of MgSO₄ of 95%. In another and/or additional preferred embodiment, said membrane comprises i) a molecular weight cut-off of 0.3 kDa, 0.4 kDa or 0.5 kDa, ii) a rejection of NaCl of 20% and iii) a rejection of MgSO₄ of 96%. In another and/or additional preferred embodiment, said membrane comprises i) a molecular weight cut-off of 0.3 kDa, 0.4 kDa or 0.5 kDa, ii) a rejection of NaCl of 20% and iii) a rejection of MgSO₄ of 97%. In another and/or additional preferred embodiment, said membrane comprises i) a molecular weight cut-off of 0.3 kDa, 0.4 kDa or 0.5 kDa, ii) a rejection of NaCl of 20% and iii) a rejection of MgSO₄ of 98%.

In another and/or additional preferred embodiment, said membrane comprises i) a molecular weight cut-off of 0.3 kDa, 0.4 kDa or 0.5 kDa, ii) a pore size of 0.001 μm, 0.0015 μm, 0.002 μm, 0.0025 μm, 0.003 μm, 0.0035 μm, 0.004 μm, 0.0045 μm, 0.005 μm, 0.0055 μm, 0.006 μm, 0.0065 μm, 0.007 μm, 0.0075 μm, 0.008 μm, 0.0085 μm, 0.009 μm, 0.0095 μm, 0.0096 μm, 0.0097 μm, 0.0098 μm, 0.0099 μm or 0.01 μm, iii) a rejection of NaCl of 20% and iv) a rejection of MgSO₄ of 94%. In another and/or additional preferred embodiment, said membrane comprises i) a molecular weight cut-off of 0.3 kDa, 0.4 kDa or 0.5 kDa, ii) a pore size of 0.001 μm, 0.0015 μm, 0.002 μm, 0.0025 μm, 0.003 μm, 0.0035 μm, 0.004 μm, 0.0045 μm, 0.005 μm, 0.0055 μm, 0.006 μm, 0.0065 μm, 0.007 μm, 0.0075 μm, 0.008 μm, 0.0085 μm, 0.009 μm, 0.0095 μm, 0.0096 μm, 0.0097 μm, 0.0098 μm, 0.0099 μm or 0.01 μm, iii) a rejection of NaCl of 20% and iv) a rejection of MgSO₄ of 95%. In another and/or additional preferred embodiment, said membrane comprises i) a molecular weight cut-off of 0.3 kDa, 0.4 kDa or 0.5 kDa, ii) a pore size of 0.001 μm, 0.0015 μm, 0.002 μm, 0.0025 μm, 0.003 μm, 0.0035 μm, 0.004 μm, 0.0045 μm, 0.005 μm, 0.0055 μm, 0.006 μm, 0.0065 μm, 0.007 μm, 0.0075 μm, 0.008 μm, 0.0085 μm, 0.009 μm, 0.0095 μm, 0.0096 μm, 0.0097 μm, 0.0098 μm, 0.0099 am or 0.01 μm, iii) a rejection of NaCl of 20% and iv) a rejection of MgSO₄ of 96%. In another and/or additional preferred embodiment, said membrane comprises i) a molecular weight cut-off of 0.3 kDa, 0.4 kDa or 0.5 kDa, ii) a pore size of 0.001 μm, 0.0015 μm, 0.002 μm, 0.0025 μm, 0.003 μm, 0.0035 μm, 0.004 μm, 0.0045 μm, 0.005 μm, 0.0055 μm, 0.006 μm, 0.0065 μm, 0.007 μm, 0.0075 μm, 0.008 μm, 0.0085 μm, 0.009 μm, 0.0095 μm, 0.0096 μm, 0.0097 μm, 0.0098 μm, 0.0099 μm or 0.01 μm, iii) a rejection of NaCl of 20% and iv) a rejection of MgSO₄ of 97%. In another and/or additional preferred embodiment, said membrane comprises i) a molecular weight cut-off of 0.3 kDa, 0.4 kDa or 0.5 kDa, ii) a pore size of 0.001 μm, 0.0015 μm, 0.002 μm, 0.0025 μm, 0.003 μm, 0.0035 μm, 0.004 μm, 0.0045 μm, 0.005 μm, 0.0055 μm, 0.006 μm, 0.0065 μm, 0.007 μm, 0.0075 μm, 0.008 μm, 0.0085 μm, 0.009 μm, 0.0095 μm, 0.0096 μm, 0.0097 μm, 0.0098 μm, 0.0099 μm or 0.01 μm, iii) a rejection of NaCl of 20% and iv) a rejection of MgSO₄ of 98%.

In another and/or additional preferred embodiment, said membrane comprises i) a molecular weight cut-off of 0.6 kDa, 0.7 kDa or 0.8 kDa, ii) a rejection of NaCl of 10% and iii) a rejection of MgSO₄ of 50%.

In another and/or additional preferred embodiment, said membrane comprises i) a molecular weight cut-off of 0.6 kDa, 0.7 kDa or 0.8 kDa, ii) a pore size of 0.001 μm, 0.0015 μm, 0.002 μm, 0.0025 μm, 0.003 μm, 0.0035 μm, 0.004 μm, 0.0045 μm, 0.005 μm, 0.0055 μm, 0.006 μm, 0.0065 μm, 0.007 μm, 0.0075 μm, 0.008 μm, 0.0085 μm, 0.009 μm, 0.0095 μm, 0.0096 μm, 0.0097 μm, 0.0098 μm, 0.0099 μm or 0.01 μm, iii) a rejection of NaCl of 10% and iv) a rejection of MgSO₄ of 50%.

In another and/or additional preferred embodiment, said membrane comprises i) a molecular weight cut-off of 0.5 kDa, 0.6 kDa or 0.7 kDa, ii) a rejection of NaCl of 40% and iii) a rejection of MgSO₄ of 95%.

In another and/or additional preferred embodiment, said membrane comprises i) a molecular weight cut-off of 0.5 kDa, 0.6 kDa or 0.7 kDa, ii) a pore size of 0.001 μm, 0.0015 μm, 0.002 μm, 0.0025 μm, 0.003 μm, 0.0035 μm, 0.004 μm, 0.0045 μm, 0.005 μm, 0.0055 μm, 0.006 μm, 0.0065 μm, 0.007 μm, 0.0075 μm, 0.008 μm, 0.0085 μm, 0.009 μm, 0.0095 μm, 0.0096 μm, 0.0097 μm, 0.0098 μm, 0.0099 μm or 0.01 μm, iii) a rejection of NaCl of 40% and iv) a rejection of MgSO₄ of 95%.

In another and/or additional preferred embodiment, said membrane comprises i) a molecular weight cut-off of 0.3 kDa, 0.4 kDa, 0.5 kDa, 0.6 kDa, 0.7 kDa, 0.8 kDa, 0.9 kDa, 1 kDa, 1.1 kDa, 1.2 kDa, 1.3 kDa, 1.4 kDa, 1.5 kDa, 1.6 kDa, 1.7 kDa, 1.8 kDa, 1.9 kDa, 2 kDa, 2.1 kDa, 2.2 kDa, 2.3 kDa, 2.4 kDa, 2.5 kDa, 2.6 kDa, 2.7 kDa, 2.8 kDa, 2.9 kDa, 3 kDa, 3.1 kDa, 3.2 kDa, 3.3 kDa, 3.4 kDa or 3.5 kDa, ii) a rejection of NaCl of 7% and iii) a rejection of MgSO₄ of 20%.

In another and/or additional preferred embodiment, said membrane comprises i) a molecular weight cut-off of 0.3 kDa, 0.4 kDa, 0.5 kDa, 0.6 kDa, 0.7 kDa, 0.8 kDa, 0.9 kDa, 1 kDa, 1.1 kDa, 1.2 kDa, 1.3 kDa, 1.4 kDa, 1.5 kDa, 1.6 kDa, 1.7 kDa, 1.8 kDa, 1.9 kDa, 2 kDa, 2.1 kDa, 2.2 kDa, 2.3 kDa, 2.4 kDa, 2.5 kDa, 2.6 kDa, 2.7 kDa, 2.8 kDa, 2.9 kDa, 3 kDa, 3.1 kDa, 3.2 kDa, 3.3 kDa, 3.4 kDa or 3.5 kDa, ii) a pore size of 0.001 μm, 0.0015 μm, 0.002 μm, 0.0025 μm, 0.003 μm, 0.0035 μm, 0.004 μm, 0.0045 μm, 0.005 μm, 0.0055 μm, 0.006 μm, 0.0065 μm, 0.007 μm, 0.0075 μm, 0.008 μm, 0.0085 μm, 0.009 μm, 0.0095 μm, 0.0096 μm, 0.0097 μm, 0.0098 μm, 0.0099 am or 0.01 μm, iii) a rejection of NaCl of 7% and iv) a rejection of MgSO₄ of 20%.

In a preferred embodiment, said membrane used in said filtration and subsequent diafiltration is chosen from the list comprising a composite membrane, a thin-film composite membrane and a zwitterionic membrane. Composite membranes consist of at least two structural elements made from different materials. Thin-film composite membranes usually comprise a selective membrane material that is deposited as a thin layer upon a porous sublayer, which serves as a support. The porous sublayer can further be supported by another layer. Each layer can be optimized independently in order to achieve the desired membrane performance. A thin-film composite membrane can, e.g., comprise a thin separation layer of 1 g polymer/m² for a 1 μm-thick selective layer. The total thickness of a thin-film composite membrane can range e.g., from about 100 μm to about 300 μm. For applying a thin top layer upon a support, one can apply any one of dip coating, spray coating, spin coating, interfacial polymerization, in situ polymerization, plasma polymerization and/or grafting. Thin-film composite membranes can e.g., be made of a thin layer of <200 nm deposited on top of a porous sublayer of about 50 am that is supported by a non-woven polyester. Thin-film membranes can comprise different nanomaterials. Zwitterionic membranes comprise zwitterions. A zwitterion is also known as an inner salt and is a molecule that has both a positively and a negatively charged group in close proximity. These charges pull water to the zwitterion while repelling organic compounds like e.g. proteins, fats and oils.

In another and/or additional preferred embodiment, said membrane used in said filtration and subsequent diafiltration is based on any one of the list comprising polypiperazine-amide, polyamide, composite polyamide, composite fluoro polymer and zwitterions.

In another and/or additional preferred embodiment, said membrane used in said filtration and subsequent diafiltration is any one of flat sheet membrane or a spiral-wound membrane.

In another and/or additional preferred embodiment, said membrane used in said filtration and subsequent diafiltration is any one of an anionic, a cationic or a zwitterionic membrane.

Examples of a membrane used in said filtration and diafiltration as described in present invention comprise UA60 membrane (Trisep), XN45 membrane (Trisep), NFW membrane (Synder), NFG membrane (Synder), NDX membrane (Synder), Z-Clear membrane (Zwitterco), GE membrane (Suez), GH membrane (Suez), GK membrane (Suez) and ETNA01PP membrane (Alfa Laval).

In another and/or additional preferred embodiment, the membrane used in the filtration and/or diafiltration of the fermentation broth has not been pre-treated with a liquid that contains any one or more of alcohol, organic sulfonic acid and sulfonate, and surfactant prior to its use in said filtration and diafiltration.

In another and/or additional preferred embodiment, the filtration and/or diafiltration is/are performed at a temperature of the fermentation broth that is subjected to said filtration and/or diafiltration ranging from 4° C. to 55° C., including 4° C. and 55° C. in the range. In a more preferred embodiment, said filtration and/or diafiltration is/are performed at a temperature of the fermentation broth ranging from 8° C. to 50° C., including 8° C. and 50° C. in the range. In an even more preferred embodiment, said filtration and/or diafiltration is/are performed at a temperature of the fermentation broth ranging from 10° C. to 45° C., including 10° C. and 45° C. in the range. In an even more preferred embodiment, said filtration and/or diafiltration is/are performed at a temperature of the fermentation broth ranging from 20° C. to 40° C., including 20° C. and 40° C. in the range. In an even more preferred embodiment, said filtration and/or diafiltration is/are performed at a temperature of the fermentation broth ranging from 4° C. to 10° C., including 4° C. and 10° C. in the range. In another more preferred embodiment, said filtration and/or diafiltration is/are performed at a temperature of the fermentation broth chosen from the list comprising about 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C. and 55° C.

In another and/or additional preferred embodiment, the filtration and/or diafiltration is/are performed using an inlet pressure ranging from 1 to 10 bar, including 1 bar and 10 bar in the range. In a more preferred embodiment, the filtration and/or diafiltration is/are performed using an inlet pressure ranging from 5 to 9.5 bar, including 5 bar and 9.5 bar in the range. In an even more preferred embodiment, the filtration and/or diafiltration is/are performed using an inlet pressure ranging from 8 to 9 bar, including 8 bar and 9 bar in the range. In another more preferred embodiment, the filtration and/or diafiltration is/are performed using an inlet pressure chosen from the list comprising 1 bar, 1.1 bar, 1.2 bar, 1.3 bar, 1.4 bar, 1.5 bar, 1.6 bar, 1.7 bar, 1.8 bar, 1.9 bar, 2 bar, 2.1 bar, 2.2 bar, 2.3 bar, 2.4 bar, 2.5 bar, 2.6 bar, 2.7 bar, 2.8 bar, 2.9 bar, 3 bar, 3.1 bar, 3.2 bar, 3.3 bar, 3.4 bar, 3.5 bar, 3.6 bar, 3.7 bar, 3.8 bar, 3.9 bar, 4 bar, 4.1 bar, 4.2 bar, 4.3 bar, 4.4 bar, 4.5 bar, 4.6 bar, 4.7 bar, 4.8 bar, 4.9 bar, 5 bar, 5.1 bar, 5.2 bar, 5.3 bar, 5.4 bar, 5.5 bar, 5.6 bar, 5.7 bar, 5.8 bar, 5.9 bar, 6 bar, 6.1 bar, 6.2 bar, 6.3 bar, 6.4 bar, 6.5 bar, 6.6 bar, 6.7 bar, 6.8 bar, 6.9 bar, 7 bar, 7.1 bar, 7.2 bar, 7.3 bar, 7.4 bar, 7.5 bar, 7.6 bar, 7.7 bar, 7.8 bar, 7.9 bar, 8 bar, 8.1 bar, 8.2 bar, 8.3 bar, 8.4 bar, 8.5 bar, 8.6 bar, 8.7 bar, 8.8 bar, 8.9 bar, 9 bar, 9.1 bar, 9.2 bar, 9.3 bar, 9.4 bar, 9.5 bar, 9.6 bar, 9.7 bar, 9.8 bar, 9.9 bar and 10 bar.

In another and/or additional preferred embodiment, the diafiltration of present invention is performed at a retentate volume of at least 50% of the original volume of the fermentation broth that is subjected to said diafiltration. In a more preferred embodiment, the diafiltration is performed at a retentate volume of at least 60% of the original volume of the fermentation broth that is subjected to said diafiltration. In an even more preferred embodiment, the diafiltration is performed at a retentate volume of at least 70% of the original volume of the fermentation broth that is subjected to said diafiltration. In an even more preferred embodiment, the diafiltration is performed at a retentate volume of at least 80% of the original volume of the fermentation broth that is subjected to said diafiltration. In an even more preferred embodiment, the diafiltration is performed at a retentate volume of at least 90% of the original volume of the fermentation broth that is subjected to said diafiltration. In an even more preferred embodiment, the diafiltration is performed at a retentate volume of at least 95% of the original volume of the fermentation broth that is subjected to said diafiltration. In another more preferred embodiment, the diafiltration is performed at a retentate volume that is of 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% of the original volume of the fermentation broth that is subjected to said diafiltration. In another more preferred embodiment, the diafiltration is performed at a retentate volume that is equal to, i.e., 100% of, the original volume of the fermentation broth that is subjected to said diafiltration.

In another and/or additional preferred embodiment, the filtration and/or diafiltration is/are performed at a permeate flux of at least 5 L/m²/h, including 5 L/m²/h, wherein said permeate flux is calculated as the volume in liter (L) of the permeate collected per hour (h) per square meter membrane area (m²) of the membrane used in said filtration and/or diafiltration. In a more preferred embodiment, the filtration and/or diafiltration is/are performed at a permeate flux of at least 6 L/m²/h, including 6 L/m²/h. In an even more preferred embodiment, the filtration and/or diafiltration is/are performed at a permeate flux of at least 7 L/m²/h, including 7 L/m²/h. In an even more preferred embodiment, the filtration and/or diafiltration is/are performed at a permeate flux of at least 8 L/m²/h, including 8 L/m²/h. In an even more preferred embodiment, the filtration and/or diafiltration is/are performed at a permeate flux of at least 9 L/m²/h, including 9 L/m²/h.

In another and/or additional preferred embodiment, the filtration and/or diafiltration is/are performed at an initial permeate flux that is at least 10% of a pure water flux, wherein:
said initial permeate flux is calculated as the volume in liter (L) of said permeate collected per hour per square meter membrane area (m²) of said membrane of said filtration and/or diafiltration,
said pure water flux is calculated as the volume in liter (L) of pure water collected per hour (h) per square meter membrane area (m²) of said membrane of said filtration and/or diafiltration, and
said initial permeate flux and said pure water flux are measured in a filtration and/or diafiltration on the same membrane under identical conditions of temperature and pressure from the start of said filtration and/or diafiltration and for at least 10 minutes, preferably for at least 20 minutes, more preferably for at least 30 minutes, even more preferably for at least 1 hour.

In a more preferred embodiment, the filtration and/or diafiltration is/are performed at an initial permeate flux that is at least 12% of a pure water flux as calculated and measured as described herein. In an even more preferred embodiment, the filtration and/or diafiltration is/are performed at an initial permeate flux that is at least 15% of a pure water flux as calculated and measured as described herein. In an even more preferred embodiment, the filtration and/or diafiltration is/are performed at an initial permeate flux that is at least 20% of a pure water flux as calculated and measured as described herein. In another more preferred embodiment, the filtration and/or diafiltration is/are performed at an initial permeate flux that is at least 12%, 13%, 14%, 15%, 16%, 17%, 18%, 19%, 20% of a pure water flux as calculated and measured as described herein.

In another and/or additional preferred embodiment, the filtration and/or diafiltration is/are performed under conditions such that the permeate flux in said filtration and/or diafiltration does not decline. In other words, the filtration and/or diafiltration is/are performed under conditions such that the decline in permeate flux in said filtration and/or diafiltration is 0%. In other words, the filtration and/or diafiltration is/are performed under conditions such that the permeate flux measured at the start of said filtration and/or diafiltration is equal to the permeate flux measured during said filtration and/or diafiltration and is equal to the permeate flux measured at the end of said filtration and/or diafiltration, when measured under identical conditions of temperature and pressure for at least 10 minutes.

In an alternative preferred embodiment, the filtration and/or diafiltration is/are performed under conditions such that the decline in permeate flux in said filtration and/or diafiltration ranges from 0 to 15%, including 0% and 15% in the range. In a more preferred embodiment, the filtration and/or diafiltration is/are performed under conditions such that the decline in permeate flux in said filtration and/or diafiltration ranges from 0 to 10%, including 0% and 10% in the range. In an even more preferred embodiment, the filtration and/or diafiltration is/are performed under conditions such that the decline in permeate flux in said filtration and/or diafiltration ranges from 0 to 5%, including 0% and 5% in the range. In an even more preferred embodiment, the filtration and/or diafiltration is/are performed under conditions such that the decline in permeate flux in said filtration and/or diafiltration ranges from 0.5 to 2.5%, including 0.5% and 2.5% in the range. In an even more preferred embodiment, the filtration and/or diafiltration is/are performed under conditions such that the decline in permeate flux in said filtration and/or diafiltration ranges from 0.1 to 1%, including 0.1% and 1% in the range. In another more preferred embodiment, the filtration and/or diafiltration is/are performed under conditions such that the decline in permeate flux in said filtration and/or diafiltration is chosen from the list comprising 0%, 0.01%, 0.05%, 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, 5%, 5.1%, 5.2%, 5.3%, 5.4%, 5.5%, 5.6%, 5.7%, 5.8%, 5.9%, 6%, 6.1%, 6.2%, 6.3%, 6.4%, 6.5%, 6.6%, 6.7%, 6.8%, 6.9%, 7%, 7.1%, 7.2%, 7.3%, 7.4%, 7.5%, 7.6%, 7.7%, 7.8%, 7.9%, 8%, 8.1%, 8.2%, 8.3%, 8.4%, 8.5%, 8.6%, 8.7%, 8.8%, 8.9%, 9%, 9.1%, 9.2%, 9.3%, 9.4%, 9.5%, 9.6%, 9.7%, 9.8%, 9.9% and 10%.

In another more preferred embodiment, the filtration and/or diafiltration is/are performed under conditions such that the permeate flux measured at the end of and/or during said filtration and/or diafiltration ranges from 90% to 100%, including 90% and 100% in the range, of the permeate flux measured at the start of said filtration and/or diafiltration, when measured under identical conditions of temperature and pressure for at least 10 minutes. In other words, the filtration and/or diafiltration is/are performed under conditions such that the permeate flux measured at the end of and/or during said filtration and/or diafiltration is any one of 90%, 90.1%, 90.2%, 90.3%, 90.4%, 90.5%, 90.6%, 90.7%, 90.8%, 90.9%, 91%, 91.1%, 91.2%, 91.3%, 91.4%, 91.5%, 91.6%, 91.7%, 91.8%, 91.9%, 92%, 92.1%, 92.2%, 92.3%, 92.4%, 92.5%, 92.6%, 92.7%, 92.8%, 92.9%, 93%, 93.1%, 93.2%, 93.3%, 93.4%, 93.5%, 93.6%, 93.7%, 93.8%, 93.9%, 94%, 94.1%, 94.2%, 94.3%, 94.4%, 94.5%, 94.6%, 94.7%, 94.8%, 94.9%, 95%, 95.1%, 95.2%, 95.3%, 95.4%, 95.5%, 95.6%, 95.7%, 95.8%, 95.9%, 96%, 96.1%, 96.2%, 96.3%, 96.4%, 96.5%, 96.6%, 96.7%, 96.8%, 96.9%, 97%, 97.1%, 97.2%, 97.3%, 97.4%, 97.5%, 97.6%, 97.7%, 97.8%, 97.9%, 98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.8%, 99.9% or 100% of the permeate flux measured at the start of said filtration and/or diafiltration, when measured under identical conditions of temperature and pressure for at least 10 minutes.

In another and/or additional preferred embodiment, the filtration and/or diafiltration is/are performed under conditions such that at the end of said filtration and/or diafiltration the surface area of said membrane is covered for 0 to 10%, including 0% and 10% in said range, with a fouling cake as described herein. In a preferred embodiment, the filtration and/or diafiltration is/are performed under conditions such that at the end of said filtration and/or diafiltration the surface area of said membrane is covered for 0.01 to 5%, including 0.01% and 5% in said range, with a fouling cake. In an even more preferred embodiment, the filtration and/or diafiltration is/are performed under conditions such that at the end of said filtration and/or diafiltration the surface area of said membrane is covered for 0.1 to 1%, including 0.1% and 1% in said range, with a fouling cake. In another more preferred embodiment, the filtration and/or diafiltration is/are performed under conditions such that at the end of said filtration and/or diafiltration the surface area of said membrane is not covered with a fouling cake. In another more preferred embodiment, the filtration and/or diafiltration is/are performed under conditions such that at the end of said filtration and/or diafiltration the surface area of said membrane is covered for 0%, 0.01%, 0.05%, 0.06%, 0.07%, 0.08%, 0.09%, 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, 5%, 5.1%, 5.2%, 5.3%, 5.4%, 5.5%, 5.6%, 5.7%, 5.8%, 5.9%, 6%, 6.1%, 6.2%, 6.3%, 6.4%, 6.5%, 6.6%, 6.7%, 6.8%, 6.9%, 7%, 7.1%, 7.2%, 7.3%, 7.4%, 7.5%, 7.6%, 7.7%, 7.8%, 7.9%, 8%, 8.1%, 8.2%, 8.3%, 8.4%, 8.5%, 8.6%, 8.7%, 8.8%, 8.9%, 9%, 9.1%, 9.2%, 9.3%, 9.4%, 9.5%, 9.6%, 9.7%, 9.8%, 9.9% or 10% with a fouling cake. In another words, the filtration and/or diafiltration is/are performed under conditions such that at the end of said filtration and/or diafiltration 0 to 10% of the surface area of said membrane is covered with a fouling cake. In another more preferred embodiment, the filtration and/or diafiltration is/are performed under conditions such that at the end of said filtration and/or diafiltration 0%, 0.01%, 0.05%, 0.06%, 0.07%, 0.08%, 0.09%, 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, 5%, 5.1%, 5.2%, 5.3%, 5.4%, 5.5%, 5.6%, 5.7%, 5.8%, 5.9%, 6%, 6.1%, 6.2%, 6.3%, 6.4%, 6.5%, 6.6%, 6.7%, 6.8%, 6.9%, 7%, 7.1%, 7.2%, 7.3%, 7.4%, 7.5%, 7.6%, 7.7%, 7.8%, 7.9%, 8%, 8.1%, 8.2%, 8.3%, 8.4%, 8.5%, 8.6%, 8.7%, 8.8%, 8.9%, 9%, 9.1%, 9.2%, 9.3%, 9.4%, 9.5%, 9.6%, 9.7%, 9.8%, 9.9% or 10% of the surface area of said membrane is covered with a fouling cake.

In another and/or additional preferred embodiment, the filtration of the fermentation broth is performed until a retentate volume is obtained of at least 50% of the original volume of said fermentation. In another more preferred embodiment, the filtration is performed until a retentate volume is obtained of at least 60% of the original volume of said fermentation. In another more preferred embodiment, the filtration is performed until a retentate volume is obtained of at least 70% of the original volume of said fermentation. In another more preferred embodiment, the filtration is performed until a retentate volume is obtained of at least 80% of the original volume of said fermentation. In another more preferred embodiment, the filtration is performed until a retentate volume is obtained of at least 90% of the original volume of said fermentation. In another more preferred embodiment, the filtration is performed until a retentate volume is obtained of at least 95% of the original volume of said fermentation. In another more preferred embodiment, the filtration is performed until a retentate volume is obtained that is of 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% of the original volume of the fermentation broth that is subjected to said filtration. In another more preferred embodiment, the filtration is performed until a retentate volume is obtained that is equal to, i.e., 100% of, the original volume of the fermentation broth. In other words, said filtration ends when a retentate volume is obtained of at least 50%, at least 60%, at least 70%, at least 80%, at least 90% and/or at least 95% of the original volume of said fermentation. Preferably, said filtration ends when a retentate volume is obtained that is of 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% of the original volume of the fermentation broth that is subjected to said filtration.

In another and/or additional preferred embodiment, the diafiltration of the fermentation broth is performed until the original constant retentate volume has been collected at least one time. In other words, the diafiltration of the fermentation broth is performed until the original constant retentate volume has been collected once, i.e., one time, or more than one time. In a more preferred embodiment, the diafiltration is performed until the original constant retentate volume has been collected at least two times, i.e., two times or more than two times. In an even more preferred embodiment, the diafiltration is performed until the original constant retentate volume has been collected at least three times, i.e., three times or more than three times. In an even more preferred embodiment, the diafiltration is performed until the original constant retentate volume has been collected at least four times, i.e., four times or more than four times. In an even more preferred embodiment, the diafiltration is performed until the original constant retentate volume has been collected at least five times, i.e., five times or more than five times. In another and/or additional preferred embodiment, the diafiltration of the fermentation broth is performed until the original retentate volume has been collected at least one time. In other words, the diafiltration of the fermentation broth is performed until the original retentate volume has been collected once, i.e., one time, or more than one time. In a more preferred embodiment, the diafiltration is performed until the original retentate volume has been collected at least two times, i.e., two times or more than two times. In an even more preferred embodiment, the diafiltration is performed until the original retentate volume has been collected at least three times, i.e., three times or more than three times. In an even more preferred embodiment, the diafiltration is performed until the original retentate volume has been collected at least four times, i.e., four times or more than four times. In an even more preferred embodiment, the diafiltration is performed until the original retentate volume has been collected at least five times, i.e., five times or more than five times. In other words, said diafiltration ends when the original retentate volume has been collected at least one time, at least two times, at least 3 times, at least 4 times and/or at least 5 times.

In another and/or additional preferred embodiment, the diafiltration of the fermentation broth is performed until a conductivity is reached for the retentate that is equal to or lower than 40 mS/cm. In a more preferred embodiment, the diafiltration of the fermentation broth is performed until a conductivity is reached for the retentate that is equal to or lower than 15 mS/cm. In an even more preferred embodiment, the diafiltration of the fermentation broth is performed until a conductivity is reached for the retentate that is equal to or lower than 10 mS/cm. In an even more preferred embodiment, the diafiltration of the fermentation broth is performed until a conductivity is reached for the retentate that is equal to or lower than 5 mS/cm. In an even more preferred embodiment, the diafiltration of the fermentation broth is performed until a conductivity is reached for the retentate that is equal to or lower than 1 mS/cm. In an even more preferred embodiment, the diafiltration of the fermentation broth is performed until a conductivity is reached for the retentate that is equal to or lower than 0.1 mS/cm. In an even more preferred embodiment, the diafiltration of the fermentation broth is performed until a conductivity is reached for the retentate that is equal to or lower than 0.01 mS/cm. In an even more preferred embodiment, the diafiltration of the fermentation broth is performed until a conductivity is reached for the retentate that is equal to or lower than 0.001 mS/cm. In another more preferred embodiment, the diafiltration of the fermentation broth is performed until a conductivity is reached for the retentate that is equal to or lower than 40 mS/cm, 39 mS/cm, 38 mS/cm, 37 mS/cm, 36 mS/cm, 35 mS/cm, 34 mS/cm, 33 mS/cm, 32 mS/cm, 31 mS/cm, 30 mS/cm, 29 mS/cm, 28 mS/cm, 27 mS/cm, 26 mS/cm, 25 mS/cm, 24 mS/cm, 23 mS/cm, 22 mS/cm, 21 mS/cm, 20 mS/cm, 19 mS/cm, 18 mS/cm, 17 mS/cm, 16 mS/cm, 15 mS/cm, 14 mS/cm, 13 mS/cm, 12 mS/cm, 11 mS/cm, 10 mS/cm, 9 mS/cm, 8 mS/cm, 7 mS/cm, 6 mS/cm, 5 mS/cm, 4 mS/cm, 3 mS/cm, 2 mS/cm, 1 mS/cm, 0.9 mS/cm, 0.8 mS/cm, 0.7 mS/cm, 0.6 mS/cm, 0.5 mS/cm, 0.4 mS/cm, 0.3 mS/cm, 0.2 mS/cm, 0.1 mS/cm, 0.09 mS/cm, 0.08 mS/cm, 0.07 mS/cm, 0.06 mS/cm, 0.05 mS/cm, 0.04 mS/cm, 0.03 mS/cm, 0.02 mS/cm, 0.01 mS/cm or 0.001 mS/cm.

In another and/or additional preferred embodiment, the membrane used in said filtration and/or diafiltration of a fermentation broth is used only once under said conditions in said method. It is to be understood herein that said membrane can also be used for filtration of another process stream in other conditions, for example, said membrane can further be used in an additional ultrafiltration and/or a nanofiltration of a further process stream from said method.

In another and/or additional preferred embodiment, the temperature of the fermentation broth is adjusted to a temperature of from 0° C. to 130° C. Said temperature adjustment can be performed at any time during said process. A temperature of from 0° C. to 130° C. should be understood as a temperature of 0° C., 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., 80° C., 81° C., 82° C., 83° C., 84° C., 85° C., 86° C., 87° C., 88° C., 89° C., 90° C., 91° C., 92° C., 93° C., 94° C., 95° C., 96° C., 97° C., 98° C., 99° C., 100° C., 101° C., 102° C., 103° C., 104° C., 105° C., 106° C., 107° C., 108° C., 109° C., 110° C., 111° C., 112° C., 113° C., 114° C., 115° C., 116° C., 117° C., 118° C., 119° C., 120° C., 121° C., 122° C., 123° C., 124° C., 125° C., 126° C., 127° C., 128° C., 129° C. or 130° C. In a more preferred embodiment, the temperature of the fermentation broth is adjusted to a temperature of from 2° C. to 122° C. A temperature of from 2° C. to 122° C. should be understood as a temperature of 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., 80° C., 81° C., 82° C., 83° C., 84° C., 85° C., 86° C., 87° C., 88° C., 89° C., 90° C., 91° C., 92° C., 93° C., 94° C., 95° C., 96° C., 97° C., 98° C., 99° C., 100° C., 101° C., 102° C., 103° C., 104° C., 105° C., 106° C., 107° C., 108° C., 109° C., 110° C., 111° C., 112° C., 113° C., 114° C., 115° C., 116° C., 117° C., 118° C., 119° C., 120° C., 121° C. or 122° C. In an even more preferred embodiment, the temperature of the solution is adjusted to a temperature of from 4° C. to 80° C. A temperature of from 4° C. to 80° C. should be understood as a temperature of 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C. or 80° C. In an even more preferred embodiment, the temperature of the solution is adjusted to a temperature of from 8° C. to 60° C. A temperature of from 8° C. to 60° C. should be understood as a temperature of 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C. or 60° C. In an even more preferred embodiment, the temperature of the solution is adjusted to a temperature of from 10° C. to 55°. A temperature of from 10° C. to 55° C. should be understood as a temperature of 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C. or 55° C. In an even more preferred embodiment, the temperature of the solution is adjusted to a temperature of from 20° C. to 45° C. A temperature of from 20° C. to 45° C. should be understood as a temperature of 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C. or 45° C. In an even more preferred embodiment, the temperature of the solution is adjusted to a temperature of from 21° C. to 40° C. A temperature of from 21° C. to 40° C. should be understood as a temperature of 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C. or 40° C. In an even more preferred embodiment, the temperature of the solution is adjusted to a temperature of from 22° C. to 37° C. A temperature of from 22° C. to 37° C. should be understood as a temperature of 22° C., 23° C., 24° C., 25° C., 26° C., 27° C., 28° C., 29° C., 30° C., 31° C., 32° C., 33° C., 34° C., 35° C., 36° C. or 37° C. In an even more preferred embodiment, the temperature of the solution is adjusted to a temperature of from 25° C. to 30° C. A temperature of from 25° C. to 30° C. should be understood as a temperature of 25° C., 26° C., 27° C., 28° C., 29° C. or 30° C.

In another and/or additional preferred embodiment, the fermentation broth is heat treated to a temperature ranging from 60° C. to 130° C. prior to said filtration. A temperature from 60° C. to 130° C. should be understood as a temperature of 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., 80° C., 81° C., 82° C., 83° C., 84° C., 85° C., 86° C., 87° C., 88° C., 89° C., 90° C., 91° C., 92° C., 93° C., 94° C., 95° C., 96° C., 97° C., 98° C., 99° C., 100° C., 101° C., 102° C., 103° C., 104° C., 105° C., 106° C., 107° C., 108° C., 109° C., 110° C., 111° C., 112° C., 113° C., 114° C., 115° C., 116° C., 117° C., 118° C., 119° C., 120° C., 121° C., 122° C., 123° C., 124° C., 125° C., 126° C., 127° C., 128° C., 129° C. or 130° C. In a more preferred embodiment, the fermentation broth is heat treated to a temperature ranging from 80° C. to 122° C. prior to said filtration.

A temperature from 80° C. to 122° C. should be understood as a temperature of 80° C., 81° C., 82° C., 83° C., 84° C., 85° C., 86° C., 87° C., 88° C., 89° C., 90° C., 91° C., 92° C., 93° C., 94° C., 95° C., 96° C., 97° C., 98° C., 99° C., 100° C., 101° C., 102° C., 103° C., 104° C., 105° C., 106° C., 107° C., 108° C., 109° C., 110° C., 111° C., 112° C., 113° C., 114° C., 115° C., 116° C., 117° C., 118° C., 119° C., 120° C., 121° C. or 122° C.

In another and/or additional preferred embodiment, the temperature of the fermentation broth is adjusted to a temperature of from 36° C. to 65° C., wherein said temperature is within 5° C. of a temperature at which the fermentation broth exhibits maximum turbidity. Said temperature adjustment can be performed at any time during said process. In a more preferred embodiment, said temperature adjustment is combined with a filtration step. In another more preferred embodiment, the temperature of the fermentation broth is adjusted to a temperature of from 36° C. to 60° C. In an even more preferred embodiment, the temperature of the fermentation broth is adjusted to a temperature of from 40° C. to 55° C. In a most preferred embodiment, the temperature of the fermentation broth is adjusted to a temperature of from 40° C. to 45° C. A temperature of from 36° C. to 65° C. should be understood as a temperature of 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C. or 65° C. A temperature of from 36° C. to 60° C. should be understood as a temperature of 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C. or 60° C. A temperature of from 40° C. to 55° C. should be understood as a temperature of 40° C., 41° C., 42° C., 43° C., 44° C., 45° C., 46° C., 47° C., 48° C., 49° C., 50° C., 51° C., 52° C., 53° C., 54° C. or 55° C. A temperature of from 40° C. to 45° C. should be understood as a temperature of 40° C., 41° C., 42° C., 43° C., 44° C. or 45° C.

In another and/or additional preferred embodiment, the pH of the fermentation broth has not been adjusted prior to the filtration of present invention. Adjustment of the pH could be obtained by one or more of addition of an acidic agent, an alkaline agent and/or a buffered solution and/or treatment of the fermentation broth by any one or more of filtration; nanofiltration; dialysis; electrodialysis; electrodeionization; ion exchange; mixed bed ion exchange; ion exchange chromatography; reverse osmosis; use of activated carbon or charcoal. pH adjustment does not comprise the acidification of said fermentation broth due to cell growth, cell lysis and/or the net negative charge of the saccharide, if said saccharide is a negatively charged saccharide, produced by the cell in said fermentation broth.

In another and/or additional preferred embodiment, the pH of the fermentation broth ranges from 2 to 7. A pH that ranges from 2 to 7 should be understood as a pH of 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9 or 7. In a more preferred embodiment, the pH of the fermentation broth ranges from 3 to 6.8. A pH that ranges from 3 to 6.8 should be understood as a pH of 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7 or 6.8. In an even more preferred embodiment, the pH of the fermentation broth ranges from 4 to 6.5. A pH that ranges from 4 to 6.5 should be understood as a pH of 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4 or 6.5. In another even more preferred embodiment, the pH of the fermentation broth ranges from 5 to 6. A pH that ranges from 5 to 6 should be understood as a pH of 5, 5.1, 5.2, 5.3, 5.4, 5.5, 5.6, 5.7, 5.8, 5.9 or 6.

In another and/or additional preferred embodiment, the biomass in the fermentation broth has a cell dry weight (CDW) that is at least 30 g/L. A CDW of at least 30 g/L is to be understood to be 30 g/L or more than 30 g/L, comprising 31 g/L, 32 g/L, 33 g/L, 34 g/L, 35 g/L, 36 g/L, 37 g/L, 38 g/L, 39 g/L, 40 g/L, 41 g/L, 42 g/L, 43 g/L, 44 g/L, 45 g/L, 46 g/L, 47 g/L, 48 g/L, 49 g/L, 50 g/L, 51 g/L, 52 g/L, 53 g/L, 54 g/L, 55 g/L, 56 g/L, 57 g/L, 58 g/L, 59 g/L, 60 g/L, 61 g/L, 62 g/L, 63 g/L, 64 g/L, 65 g/L, 66 g/L, 67 g/L, 68 g/L, 69 g/L, 70 g/L, 71 g/L, 72 g/L, 73 g/L, 74 g/L, 75 g/L, 76 g/L, 77 g/L, 78 g/L, 79 g/L, 80 g/L, 81 g/L, 82 g/L, 83 g/L, 84 g/L, 85 g/L, 86 g/L, 87 g/L, 88 g/L, 89 g/L, 90 g/L, 91 g/L, 92 g/L, 93 g/L, 94 g/L, 95 g/L, 96 g/L, 97 g/L, 98 g/L, 99 g/L, 100 g/L and more than 100 g/L. In a preferred embodiment, the biomass in the fermentation broth has a CDW that is at least 40 g/L. A CDW of at least 40 g/L is to be understood to be 40 g/L or more than 40 g/L, comprising 41 g/L, 42 g/L, 43 g/L, 44 g/L, 45 g/L, 46 g/L, 47 g/L, 48 g/L, 49 g/L, 50 g/L, 51 g/L, 52 g/L, 53 g/L, 54 g/L, 55 g/L, 56 g/L, 57 g/L, 58 g/L, 59 g/L, 60 g/L, 61 g/L, 62 g/L, 63 g/L, 64 g/L, 65 g/L, 66 g/L, 67 g/L, 68 g/L, 69 g/L, 70 g/L, 71 g/L, 72 g/L, 73 g/L, 74 g/L, 75 g/L, 76 g/L, 77 g/L, 78 g/L, 79 g/L, 80 g/L, 81 g/L, 82 g/L, 83 g/L, 84 g/L, 85 g/L, 86 g/L, 87 g/L, 88 g/L, 89 g/L, 90 g/L, 91 g/L, 92 g/L, 93 g/L, 94 g/L, 95 g/L, 96 g/L, 97 g/L, 98 g/L, 99 g/L, 100 g/L and more than 100 g/L. In a more preferred embodiment, the biomass in the fermentation broth has a CDW that is at least 50 g/L. A CDW of at least 50 g/L is to be understood to be 50 g/L or more than 50 g/L, comprising 51 g/L, 52 g/L, 53 g/L, 54 g/L, 55 g/L, 56 g/L, 57 g/L, 58 g/L, 59 g/L, 60 g/L, 61 g/L, 62 g/L, 63 g/L, 64 g/L, 65 g/L, 66 g/L, 67 g/L, 68 g/L, 69 g/L, 70 g/L, 71 g/L, 72 g/L, 73 g/L, 74 g/L, 75 g/L, 76 g/L, 77 g/L, 78 g/L, 79 g/L, 80 g/L, 81 g/L, 82 g/L, 83 g/L, 84 g/L, 85 g/L, 86 g/L, 87 g/L, 88 g/L, 89 g/L, 90 g/L, 91 g/L, 92 g/L, 93 g/L, 94 g/L, 95 g/L, 96 g/L, 97 g/L, 98 g/L, 99 g/L, 100 g/L and more than 100 g/L. In an even more preferred embodiment, the biomass in the fermentation broth has a CDW that is at least 60 g/L. A CDW of at least 60 g/L is to be understood to be 60 g/L or more than 60 g/L, comprising 61 g/L, 62 g/L, 63 g/L, 64 g/L, 65 g/L, 66 g/L, 67 g/L, 68 g/L, 69 g/L, 70 g/L, 71 g/L, 72 g/L, 73 g/L, 74 g/L, 75 g/L, 76 g/L, 77 g/L, 78 g/L, 79 g/L, 80 g/L, 81 g/L, 82 g/L, 83 g/L, 84 g/L, 85 g/L, 86 g/L, 87 g/L, 88 g/L, 89 g/L, 90 g/L, 91 g/L, 92 g/L, 93 g/L, 94 g/L, 95 g/L, 96 g/L, 97 g/L, 98 g/L, 99 g/L, 100 g/L and more than 100 g/L. In an even more preferred embodiment, the biomass in the fermentation broth has a CDW that is at least 70 g/L. A CDW of at least 70 g/L is to be understood to be 70 g/L or more than 70 g/L, comprising 71 g/L, 72 g/L, 73 g/L, 74 g/L, 75 g/L, 76 g/L, 77 g/L, 78 g/L, 79 g/L, 80 g/L, 81 g/L, 82 g/L, 83 g/L, 84 g/L, 85 g/L, 86 g/L, 87 g/L, 88 g/L, 89 g/L, 90 g/L, 91 g/L, 92 g/L, 93 g/L, 94 g/L, 95 g/L, 96 g/L, 97 g/L, 98 g/L, 99 g/L, 100 g/L and more than 100 g/L. In an even more preferred embodiment, the biomass in the fermentation broth has a CDW that is at least 80 g/L. A CDW of at least 80 g/L is to be understood to be 80 g/L or more than 80 g/L, comprising 81 g/L, 82 g/L, 83 g/L, 84 g/L, 85 g/L, 86 g/L, 87 g/L, 88 g/L, 89 g/L, 90 g/L, 91 g/L, 92 g/L, 93 g/L, 94 g/L, 95 g/L, 96 g/L, 97 g/L, 98 g/L, 99 g/L, 100 g/L and more than 100 g/L. In an even more preferred embodiment, the biomass in the fermentation broth has a CDW that is at least 90 g/L. A CDW of at least 90 g/L is to be understood to be 90 g/L or more than 90 g/L, comprising 91 g/L, 92 g/L, 93 g/L, 94 g/L, 95 g/L, 96 g/L, 97 g/L, 98 g/L, 99 g/L, 100 g/L and more than 100 g/L. In an even more preferred embodiment, the biomass in the fermentation broth has a CDW that is at least 100 g/L. A CDW of at least 100 g/L is to be understood to be 100 g/L or more than 100 g/L.

In a specific embodiment, fermentation broth comprising saccharide and biomass is subjected to filtration and diafiltration, wherein said biomass is retained in the retentate. In a preferred embodiment, said fermentation broth further comprises antifoam and/or proteins. In another preferred embodiment, said antifoam and/or proteins are retained in the retentate after passing said fermentation broth to said filtration and diafiltration.

In another and/or additional preferred embodiment, the fermentation broth is not subjected to dilution prior to the filtration of present invention.

In another specific embodiment, the saccharide present in said fermentation broth passes through the membrane of said filtration and diafiltration, enabling the collection of said saccharide in the permeate.

In another and/or additional preferred embodiment, the method further comprises any one or more of concentration, homogenization, clarification, clearing, centrifugation, decantation, dilution, pH adjustment, temperature adjustment, filtration, ultrafiltration, microfiltration, diafiltration, reverse osmosis, electrodialysis, electrodeionization, nanofiltration, dialysis, use of activated charcoal or carbon, use of solvents, use of alcohols, use of aqueous alcohol mixtures, use of charcoal, tangential flow high-performance filtration, tangential flow ultrafiltration, affinity chromatography, ion exchange, ion exchange chromatography, mixed bed ion exchange, hydrophobic interaction chromatography, gel filtration, ligand exchange chromatography, column chromatography, cation exchange adsorbent resin, anion exchange adsorbent resin, use of an adsorbent material, use of ion exchange resin, evaporation, vacuum evaporation, wiped film evaporation, falling film evaporation, pasteurization, enzymatic treatment, decolorization and drying, in any order. In a more preferred embodiment, any or more of said methods is performed more than one time during said method.

In a preferred embodiment, the method further comprises ultrafiltration and/or nanofiltration. In another preferred embodiment, the method further comprises ultrafiltration, nanofiltration and electrodialysis. In another preferred embodiment, the method further comprises ultrafiltration, nanofiltration and electrodeionization. In another preferred embodiment, the method further comprises ultrafiltration, nanofiltration, electrodialysis and electrodeionization. In another preferred embodiment, the method does not further comprise electrodialysis. In another preferred embodiment, the method does not further comprise electrodeionization. In another preferred embodiment, the method further comprises nanofiltration, use of activated charcoal and ion exchange chromatography. In another preferred embodiment, the method further comprises nanofiltration, use of activated charcoal, microfiltration and ion exchange chromatography. In another preferred embodiment, the method further comprises mixed bed ion exchange comprising a cationic ion exchange resin and an anionic ion exchange resin, wherein said cationic ion exchange resin is in any form chosen from the list comprising Na⁺, K⁺, Ca²⁺, Mg²⁺, Al³⁺, NH₄⁺ and wherein said anionic ion exchange resin is in any form chosen from the list comprising OH—, Cl⁻ and SO₃²⁺. In another preferred embodiment, the method comprises two mixed bed ion exchanges wherein the cationic ion exchange resins present in both mixed bed ion exchanges are in H+ form and wherein the anionic ion exchange resins present in both mixed bed ion exchanges are in OH— form.

In another and/or additional preferred embodiment, the method further comprises clarification, preferably wherein said clarification is performed by any one or more of microfiltration, centrifugation, flocculation or ultrafiltration.

In another and/or additional preferred embodiment, the method further comprises use of a cation exchange adsorbent resin, an anion exchange adsorbent resin and/or use of an adsorbent material.

In another and/or additional preferred embodiment, the method further comprises drying, preferably wherein said drying is chosen from the list comprising spray drying, lyophilization, spray freeze drying, freeze spray drying, band drying, belt drying, vacuum band drying, vacuum belt drying, drum drying, roller drying, vacuum drum drying, vacuum roller drying, and agitated thin film drying.

In another and/or additional preferred embodiment, the method further comprises filtration, preferably wherein said filtration is performed by use of a filtration aid and/or flocculant. Preferably said filtration aid is an adsorbing agent, more preferably said filtration aid is active carbon.

In another and/or additional preferred embodiment, the method further comprises ultrafiltration, preferably wherein said ultrafiltration has a molecular weight cut-off equal to or higher than 1 kDa, 2 kDa, 3 kDa, 4 kDa, 5 kDa, 6 kDa, 7 kDa, 8 kDa, 9 kDa, 10 kDa, 11 kDa, 12 kDa, 13 kDa, 14 kDa, 15 kDa.

In another and/or additional preferred embodiment, the solution is subjected to two consecutive ultrafiltration steps, preferably wherein the membrane molecular weight cut-off used in the first ultrafiltration step is higher than that used in the second ultrafiltration step.

In another and/or additional preferred embodiment, the method further comprises nanofiltration, preferably wherein the nanofiltration membrane used in said nanofiltration has a size exclusion limit of 20 Å, in other words said nanofiltration has a size exclusion limit of 1 Å, 2 Å, 3 Å, 4 Å, 5 Å, 6 Å, 7 Å, 8 Å, 9 Å, 10 Å, 11 Å, 12 Å, 13 Å, 14 Å, 15 Å, 16 Å, 17 Å, 18 Å, 19 Å or 20 Å.

In another and/or additional preferred embodiment, the method further comprises nanofiltration, preferably wherein said nanofiltration is performed at a pressure ranging from 5 to 45 bar. In other words, said nanofiltration is performed at a pressure of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44 or 45 bar.

In another and/or additional preferred embodiment, the method further comprises diafiltration, preferably wherein said diafiltration is performed on said solution until a conductivity is reached of 40 mS/cm, preferably 15 mS/cm, 10 mS/cm, 5 mS/cm, 1 mS/cm, 0.1 mS/cm, <0.01 mS/cm, 0.001 mS/cm. In a more preferred embodiment, the method further comprises diafiltration, wherein said diafiltration is performed on the solution until a conductivity is reached of any one of 15, 14, 13, 12, 11, 10, 9, 8, 7, 6, 5, 4, 3, 2 or 1 mS/cm.

In another and/or additional preferred embodiment, the method further comprises microfiltration, preferably wherein the pore openings in the membrane used in the microfiltration are ranging from 0.1 to 1 μm (micron).

In another and/or additional preferred embodiment, the method further comprises ultrafiltration, preferably wherein the pore openings in the membrane used in the ultrafiltration are ranging from 0.01 to 0.1 μm (micron).

In another and/or additional preferred embodiment, the method further comprises nanofiltration, preferably wherein the pore openings in the membrane used in the nanofiltration are ranging from 0.001 to 0.01 μm (micron).

In another and/or additional preferred embodiment, the method further comprises reverse osmosis, preferably wherein the pore openings in the membrane used in the reverse osmosis are ranging from 0.0001 to 0.001 μm (micron).

In another and/or additional preferred embodiment, the method further comprises an enzymatic treatment, preferably wherein the enzymatic treatment comprises incubation with one or more enzymes selected from the group comprising glycosidase, lactase, β-galactosidase, fucosidase, sialidase, maltase, amylase, hexaminidase, glucuronidase, trehalase, and invertase.

In another and/or additional preferred embodiment, the method further comprises an enzymatic treatment, preferably wherein the enzymatic treatment converts lactose, sucrose, malto-oligosaccharides, maltotriose, sorbitol, trehalose, starch, cellulose, hemi-cellulose, lignocellulose, molasses, corn-steep liquor and/or high-fructose syrup to monosaccharides.

In another and/or additional preferred embodiment, the method is a batch process. In an alternative and/or additional preferred embodiment, the method is a continuous process.

In another and/or additional preferred embodiment of the present invention, the purity of the saccharide obtained in a purified saccharide solution at the end of the method is at least 70%, at least 75%, at least 80%, at least 85%, at least 90%, at least 95%, at least 97%, at least 98%, at least 99% on total dry solid. At least 70% should be understood as 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. At least 75% should be understood as 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. At least 80% should be understood as 80%, 81%, 82%, 83%, 84%, 85%, 86%, 87%, 88%, 89%, 90%, 91%, 92%, 93%, 94%, 95%, 96%, 97%, 98%, 99% or 100%. At least 85% should be understood as 85%, 86%, 87%, 88%, 89%, 90%, 91%, 92%, 93%, 94%, 95%, 96%, 97%, 98%, 99% or 100%. At least 90% should be understood as 90%, 91%, 92%, 93%, 94%, 95%, 96%, 97%, 98%, 99% or 100%. At least 95% should be understood as 95%, 95.5%, 96%, 96.5%, 97%, 97.5%, 98%, 98.5%, 99%, 99.5% or 100%. At least 97% should be understood as 97%, 97.5%, 98%, 98.5%, 99%, 99.5% or 100%. At least 98% should be understood as 98%, 98.5%, 99%, 99.5% or 100%.

At least 99% should be understood as 99%, 99.1%, 99.2%, 99.3%, 99.4%, 99.5%, 99.6%, 99.7%, 99.8%, 99.9% or 100%.

In another and/or additional preferred embodiment of the present invention, the yield of purification of the saccharide obtained in the purified saccharide solution at the end of the method is at least 60%, at least 65%, at least 70%, at least 75%, at least 80%, at least 85%, at least 90%, at least 95%, at least 97%, at least 98%, at least 99%. At least 60% should be understood as 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. At least 65% should be understood as 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. At least 70% should be understood as 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. At least 75% should be understood as 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. At least 80% should be understood as 80%, 81%, 82%, 83%, 84%, 85%, 86%, 87%, 88%, 89%, 90%, 91%, 92%, 93%, 94%, 95%, 96%, 97%, 98%, 99% or 100%. At least 85% should be understood as 85%, 86%, 87%, 88%, 89%, 90%, 91%, 92%, 93%, 94%, 95%, 96%, 97%, 98%, 99% or 100%. At least 90% should be understood as 90%, 91%, 92%, 93%, 94%, 95%, 96%, 97%, 98%, 99% or 100%. At least 95% should be understood as 95%, 95.5%, 96%, 96.5%, 97%, 97.5%, 98%, 98.5%, 99%, 99.5% or 100%. At least 97% should be understood as 97%, 97.5%, 98%, 98.5%, 99%, 99.5% or 100%. At least 98% should be understood as 98%, 98.5%, 99%, 99.5% or 100%.

At least 99% should be understood as 99%, 99.1%, 99.2%, 99.3%, 99.4%, 99.5%, 99.6%, 99.7%, 99.8%, 99.9% or 100%.

In another and/or additional preferred embodiment of the present invention, the purified saccharide solution obtained at the end of the method has an ash content of 10% on total dry solid, preferably <9% on total dry solid, more preferably 8% on total dry solid, even more preferably 7% on total dry solid, even more preferably 6% on total dry solid, even more preferably 5% on total dry solid. In a more preferred embodiment, the purified saccharide solution obtained at the end of the method has an ash content of any one of 10%, 9%, 8%, 6%, 5%, 4%, 3%, 2%, 1%, 0.5%, 0.4%, 0.3%, 0.2% or 0.1% on total dry solid.

In another and/or additional preferred embodiment of the present invention, the purified saccharide solution obtained at the end of the method has an ash content of 10% on total dry solid, preferably with a lead content lower than 0.1 mg/kg dry solid, an arsenic content lower than 0.2 mg/kg dry solid, a cadmium content lower than 0.1 mg/kg dry solid and/or a mercury content lower than 0.5 mg/kg dry solid.

In another and/or additional preferred embodiment, the purified saccharide solution obtained at the end of the method has a lead content lower than 0.1 mg/kg dry solid, more preferably lower than 0.05 mg/kg dry solid, even more preferably below 0.02 mg/kg dry solid. In another and/or additional preferred embodiment, the purified saccharide solution obtained at the end of the method has an arsenic content lower than 0.2 mg/kg dry solid, more preferably lower dan 0.1 mg/kg dry solid, even more preferably lower than 0.05 mg/kg dry solid. In another and/or additional preferred embodiment, the purified saccharide solution obtained at the end of the method has a cadmium content lower than 0.1 mg/kg dry solid, more preferably lower than 0.05 mg/kg dry solid, even more preferably below 0.02 mg/kg dry solid. In another and/or additional preferred embodiment, the purified saccharide solution obtained at the end of the method has a mercury content lower than 0.5 mg/kg dry solid, more preferably lower than 0.2 mg/kg dry solid, even more preferably below 0.1 mg/kg dry solid.

In another and/or additional preferred embodiment, the purified saccharide solution obtained at the end of the method is filter-sterilized. In another and/or additional preferred embodiment, the purified saccharide solution obtained at the end of the method is subjected to endotoxin removal. Preferably, endotoxin removal is performed by filtration through a 3 kDa filter, i.e., filtration with a membrane having a molecular weight cut-off of 3 kDa.

In another and/or additional preferred embodiment, the purified saccharide solution obtained at the end of the method has a protein content below 100 mg per kg dry solid, a DNA content below 10 ng per gram dry solid and/or an endotoxin content below 10000 EU per gram dry solid. In a more preferred embodiment, the purified saccharide solution obtained at the end of the method has a protein content equal to or below 99 mg per kg dry solid, equal to or below 95 mg per kg dry solid, equal to or below 90 mg per kg dry solid, equal to or below 80 mg per kg dry solid, equal to or below 70 mg per kg dry solid, equal to or below 60 mg per kg dry solid, equal to or below 50 mg per kg dry solid, equal to or below 40 mg per kg dry solid, equal to or below 30 mg per kg dry solid, equal to or below 20 mg per kg dry solid or equal to or below 10 mg per kg dry solid. In another and/or additional more preferred embodiment, the purified saccharide solution obtained at the end of the method has a DNA content equal to or below 9 ng per gram dry solid, equal to or below 8 ng per gram dry solid, equal to or below 7 ng per gram dry solid, equal to or below 6 ng per gram dry solid, equal to or below 5 ng per gram dry solid, equal to or below 4 ng per gram dry solid, equal to or below 3 ng per gram dry solid, equal to or below 2 ng per gram dry solid, equal to or below 1 ng per gram dry solid, or no DNA. In another and/or additional more preferred embodiment, the purified saccharide solution obtained at the end of the method has an endotoxin content equal to or below 8000 EU per gram dry solid, equal to or below 5000 EU per gram dry solid, equal to or below 3000 EU per gram dry solid, equal to or below 1000 EU per gram dry solid, equal to or below 800 EU per gram dry solid, equal to or below 500 EU per gram dry solid, equal to or below 300 EU per gram dry solid or equal to or below 100 EU per gram dry solid. In an even more preferred embodiment, the purified saccharide solution obtained at the end of the method is free of DNA, proteins, and/or recombinant genetic material. In another more preferred embodiment, the purified saccharide solution obtained at the end of the method is free of recombinant DNA and/or proteins derived from the recombinant micro-organism.

In another and/or additional preferred embodiment, the purified saccharide solution obtained at the end of the method has less than 125 μg/kg of 3-MCPD, wherein said 3-MCPD is the sum of 3-monochloropropanediol (3-MCPD) and 3-MCPD fatty acid esters.

In another and/or additional preferred embodiment, the purified saccharide solution obtained at the end of the method has less than 0.5 μg/kg epichlorohydrin.

In another and/or additional preferred embodiment, the purified saccharide solution obtained at the end of the method has a conductivity of less than 10 mS/cm at a 300 g/L solution.

In another and/or additional preferred embodiment, the purified saccharide solution obtained at the end of the method is further concentrated. Concentration can be performed by means of one or more of nanofiltration, diafiltration, reverse osmosis, evaporation, vacuum evaporation, wiped film evaporation, and falling film evaporation. In another preferred embodiment, the method further comprises any one or more of nanofiltration, diafiltration, reverse osmosis, evaporation, vacuum evaporation, wiped film evaporation, and falling film evaporation, wherein one or more of said nanofiltration, diafiltration, reverse osmosis, evaporation, vacuum evaporation, wiped film evaporation, and falling film evaporation is performed more than one time during the process.

In another and/or additional preferred embodiment, the purified saccharide solution obtained at the end of the method is further concentrated to a syrup of at least 20% dry matter. In a more preferred embodiment, the purified saccharide solution obtained at the end of the method is further concentrated to a syrup of at least 30% dry matter. In a more preferred embodiment, the purified saccharide solution obtained at the end of the method is further concentrated to a syrup of at least 40% dry matter.

In another and/or additional preferred embodiment, the purified saccharide solution obtained at the end of the method is first subjected to a polishing step prior to concentration. For this polishing step, an adsorbent material, such as activated carbon or charcoal, a cation exchange adsorbent resin, an anion exchange adsorbent resin or a charge-modified depth filter can be used.

In another and/or additional preferred embodiment, the purified saccharide solution obtained at the end of the method is further crystallised. In another and/or additional preferred embodiment, the purified saccharide solution obtained at the end of the method is further dried to a powder. In another and/or additional preferred embodiment, the purified saccharide solution obtained at the end of the method is further granulated.

In another and/or additional preferred embodiment, the purified saccharide solution obtained at the end of the method is further concentrated by a method to a saccharide concentration of >100 g/L, preferably >200 g/L, more preferably >300 g/L, more preferably >400 g/L, more preferably >500 g/L, more preferably >600 g/L, most preferably between 300 g/L and 650 g/L. Preferably, said concentration is performed at a temperature of <80° C., preferably <60° C., more preferably <50° C., more preferably 20° C. to 50° C., even more preferably 30° C. to 45° C. Herein, 20° C. to 50° C. is to be understood as 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C. or 50° C. Herein, 30° C. to 45° C. is to be understood as 30° C., 31° C., 32° C., 33° C., 34° C., 35° C., 36° C., 37° C., 38° C., 39° C., 40° C., 41° C., 42° C., 43° C., 44° C. or 45° C. Preferably, any of said concentration method is chosen from the list comprising using vacuum evaporation or reverse osmosis or nanofiltration.

In another and/or additional preferred embodiment, the purified saccharide solution obtained at the end of the method comprises a saccharide which is concentrated to a concentration of >1.5 M and cooled to a temperature <25° C., more preferably <8° C., to obtain crystalline material of the saccharide. A temperature <25° C. is to be understood as 0° C., 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C. or a temperature below 0° C. A temperature of <8° C. is to be understood as 0° C., 1° C., 2° C., 3° C., 4° C., 5° C., 6° C., 7° C. or 8° C. or a temperature below 0° C.

In another and/or additional preferred embodiment, the purified saccharide solution obtained at the end of the method has a Brix value of from about 8 to about 75%, preferably the purified saccharide solution has a Brix value of from about 30 to about 65%.

In another and/or additional preferred embodiment, the purified saccharide solution obtained at the end of the method is dried by any one or more of drying steps chosen from the list comprising spray drying, lyophilization, evaporation, precipitation, spray freeze drying, freeze spray drying, band drying, belt drying, vacuum band drying, vacuum belt drying, drum drying, roller drying, vacuum drum drying, vacuum roller drying and agitated thin film drying. In a more preferred embodiment, the purified saccharide solution obtained at the end of the method is dried by spray-drying, freeze-drying or agitated thin film drying. In an additional more preferred embodiment, the pH of the purified saccharide solution is ranging from 2 to 7. In other words, the pH of the purified saccharide solution is any one of 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9 or 7. In an additional even more preferred embodiment, the pH of the purified saccharide solution is ranging from 3 to 6; in other words, the pH of the purified saccharide solution is any one of 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9 or 6. In an additional even more preferred embodiment, the pH of the purified saccharide solution is ranging from 4 to 5; in other words, the pH of the purified saccharide solution is any one of 4, 4.1, 4.2, 4.3, 4.4, 4.5, 4.6, 4.7, 4.8, 4.9 or 5. In another more preferred embodiment, the purified saccharide solution obtained at the end of the method is dried by spray-drying, particularly spray-dried at a saccharide solution concentration of 20-60 (w/v), preferably 30-50 (w/v), more preferably 35-45 (w/v), with a nozzle temperature of 110-150° C., preferably 120-140° C., more preferably 125-135° C. and/or an outlet temperature of 60-80° C., preferably 65-70° C.

According to a second aspect, the present invention provides a purified saccharide solution, a purified saccharide or a purified saccharide mixture comprising a saccharide obtainable, preferably obtained, by a method as described herein. The purified saccharide solution can comprise one purified saccharide or a purified saccharide mixture.

In a preferred and/or additional embodiment, the present invention provides a purified saccharide obtainable, preferably obtained, by a method as described herein, wherein the purified saccharide solution comprising said purified saccharide is dried. In a more preferred embodiment, the present invention provides a purified saccharide obtainable, preferably obtained, by a method as described herein, wherein the purified saccharide solution comprising said purified saccharide is spray-dried. In an alternative more preferred embodiment, the present invention provides a purified saccharide obtainable, preferably obtained, by a method as described herein, wherein the purified saccharide solution comprising said purified saccharide is dried via an agitated thin film dryer.

In another and/or additional preferred embodiment, the present invention provides a purified saccharide obtainable, preferably obtained, by a method as described herein, wherein the purified saccharide solution comprising said purified saccharide is lyophilized.

In another and/or additional preferred embodiment, the present invention provides a purified saccharide obtainable, preferably obtained, by a method as described herein, wherein the purified saccharide solution comprising said purified saccharide is crystallized.

In another and/or additional preferred embodiment, the present invention provides a purified saccharide obtainable, preferably obtained, by a method as described herein, wherein the purified saccharide solution comprising said purified saccharide is concentrated to a syrup of at least 20% dry matter. In a more preferred embodiment, the present invention provides a purified saccharide obtainable, preferably obtained, by a method as described herein, wherein the purified saccharide solution comprising said purified saccharide is concentrated to a syrup of at least 30% dry matter. In an even more preferred embodiment, the present invention provides a purified saccharide obtainable, preferably obtained, by a method as described herein, wherein the purified saccharide solution comprising said purified saccharide is concentrated to a syrup of at least 40% dry matter.

In another and/or additional preferred embodiment, the present invention provides a purified saccharide mixture comprising a saccharide obtainable, preferably obtained, by a method as described herein, wherein the purified saccharide solution comprising said purified saccharide mixture comprising a saccharide is dried. In a more preferred embodiment, the present invention provides a purified saccharide mixture comprising a saccharide obtainable, preferably obtained, by a method as described herein, wherein the purified saccharide solution comprising said purified saccharide mixture comprising a saccharide is spray-dried. In an alternative more preferred embodiment, the present invention provides a purified saccharide mixture comprising a saccharide obtainable, preferably obtained, by a method as described herein, wherein the purified saccharide solution comprising said purified saccharide mixture comprising a saccharide is dried via an agitated thin film dryer.

In another and/or additional preferred embodiment, the present invention provides a purified saccharide mixture comprising a saccharide obtainable, preferably obtained, by a method as described herein, wherein the purified saccharide solution comprising said purified saccharide mixture comprising a saccharide is lyophilized.

In another and/or additional preferred embodiment, the present invention provides a purified saccharide mixture comprising a saccharide obtainable, preferably obtained, by a method as described herein, wherein the purified saccharide solution comprising said purified saccharide mixture comprising a saccharide is crystallized.

In another and/or additional preferred embodiment, the present invention provides a purified saccharide mixture comprising a saccharide obtainable, preferably obtained, by a method as described herein, wherein the purified saccharide solution comprising said purified saccharide mixture comprising a saccharide is concentrated to a syrup of at least 20% dry matter. In a more preferred embodiment, the present invention provides a purified saccharide mixture comprising a saccharide obtainable, preferably obtained, by a method as described herein, wherein the purified saccharide solution comprising said purified saccharide mixture comprising a saccharide is concentrated to a syrup of at least 30% dry matter. In an even more preferred embodiment, the present invention provides a purified saccharide mixture comprising a saccharide obtainable, preferably obtained, by a method as described herein, wherein the purified saccharide solution comprising said purified saccharide mixture comprising a saccharide is concentrated to a syrup of at least 40% dry matter.

In another and/or additional preferred embodiment, the present invention provides a saccharide that is purified according to a method as described herein and that contains less than 10% ash after said method. Less than 10% ash is to be understood as 10%, 9%, 8%, 7%, 6%, 5%, 4%, 3%, 2%, 1%, 0.5% or less than 0.5% ash.

In another and/or additional preferred embodiment, the present invention provides a saccharide that is purified according to a process as described herein and that has an ash content of 10% on total dry solid, wherein said ash comprises any one or more of a heavy metal selected from the list comprising lead, arsenic, cadmium, mercury, zinc, manganese, copper, iron, magnesium and calcium.

In another and/or additional preferred embodiment, the present invention provides a saccharide that is purified according to a process as described herein and that has a lead content <0.1 mg/kg dry solid. In a more preferred embodiment, said purified saccharide has a lead content <0.02 mg/kg dry solid. In an even more preferred embodiment, said purified saccharide has a lead content <0.01 mg/kg dry solid. In another and/or additional preferred embodiment, the present invention provides a saccharide that is purified according to a process as described herein and that has an arsenic content <0.2 mg/kg dry solid. In a more preferred embodiment, said purified saccharide has an arsenic content <0.05 mg/kg dry solid. In an even more preferred embodiment, said purified saccharide has an arsenic content <0.02 mg/kg dry solid.

In another and/or additional preferred embodiment, the present invention provides a saccharide that is purified according to a process as described herein and that has a cadmium content <0.1 mg/kg dry solid. In a more preferred embodiment, said purified saccharide has a cadmium content <0.01 mg/kg dry solid. In another and/or additional preferred embodiment, the present invention provides a saccharide that is purified according to a process as described herein and that has a mercury content <0.5 mg/kg dry solid. In a more preferred embodiment, said purified saccharide has a mercury content <0.1 mg/kg dry solid. In an even more preferred embodiment, said purified saccharide has a mercury content <0.005 mg/kg dry solid.

In another and/or additional preferred embodiment, the present invention provides a saccharide that is purified according to a process as described herein and that has protein content below 100 mg per kg dry solid, a DNA content below 10 ng per gram dry solid and/or an endotoxin content below 10000 EU per gram dry solid. In a more preferred embodiment, the present invention provides a saccharide that is purified according to a process as described herein and that has a protein content equal to or below 99 mg per kg dry solid, equal to or below 95 mg per kg dry solid, equal to or below 90 mg per kg dry solid, equal to or below 80 mg per kg dry solid, equal to or below 70 mg per kg dry solid, equal to or below 60 mg per kg dry solid, equal to or below 50 mg per kg dry solid, equal to or below 40 mg per kg dry solid, equal to or below 30 mg per kg dry solid, equal to or below 20 mg per kg dry solid or equal to or below 10 mg per kg dry solid. In another and/or additional more preferred embodiment, the present invention provides a saccharide that is purified according to a process as described herein and that has a DNA content equal to or below 9 ng per gram dry solid, equal to or below 8 ng per gram dry solid, equal to or below 7 ng per gram dry solid, equal to or below 6 ng per gram dry solid, equal to or below 5 ng per gram dry solid, equal to or below 4 ng per gram dry solid, equal to or below 3 ng per gram dry solid, equal to or below 2 ng per gram dry solid, equal to or below 1 ng per gram dry solid, or no DNA. In another and/or additional more preferred embodiment, the present invention provides a saccharide that is purified according to a process as described herein and that has an endotoxin content equal to or below 8000 EU per gram dry solid, equal to or below 5000 EU per gram dry solid, equal to or below 3000 EU per gram dry solid, equal to or below 1000 EU per gram dry solid, equal to or below 800 EU per gram dry solid, equal to or below 500 EU per gram dry solid, equal to or below 300 EU per gram dry solid or equal to or below 100 EU per gram dry solid. In an even more preferred embodiment, the present invention provides a saccharide that is purified according to a process as described herein and that is free of DNA, recombinant DNA, proteins, proteins derived from the recombinant micro-organism and/or recombinant genetic material.

In another and/or additional preferred embodiment, the present invention provides a saccharide that is purified according to a process as described herein and that has less than 125 μg/kg of 3-MCPD, wherein said 3-MCPD is the sum of 3-monochloropropanediol (3-MCPD) and 3-MCPD fatty acid esters.

In another and/or additional preferred embodiment, the present invention provides a saccharide that is purified according to a process as described herein and that has less than 0.5 μg/kg epichlorohydrin.

In another and/or additional preferred embodiment, the present invention provides a saccharide that is purified according to a process as described herein and that has a conductivity of less than 10 mS/cm at a 300 g/L solution.

According to a third aspect, the present invention provides a spray-dried saccharide or a spray-dried saccharide mixture comprising a saccharide, wherein said saccharide or saccharide mixture is purified according to a method as described herein and wherein said spray-dried saccharide or spray-dried saccharide mixture obtained after said method contains less than 10% ash. Less than 10% ash is to be understood as 10%, 9%, 8%, 7%, 6%, 5%, 4%, 3%, 2%, 1%, 0.5% or less than 0.5% ash.

According to another aspect, the present invention provides a dried powder of purified saccharide solution obtained from a method as described herein, wherein said dried powder contains ≤15%-wt. of water. A dried powder containing ≤15%-wt. of water is to be understood as a dried powder containing 15%-wt., 14%-wt., 13%-wt., 12%-wt., 11%-wt., 10%-wt., 9%-wt., 8%-wt., 7%-wt., 6%-wt., 5%-wt., 4%-wt., 3%-wt., 2%-wt., 1%-wt., 0.5%-wt., 0.4%-wt., 0.3%-wt., 0.2%-wt., 0.1%-wt. or 0%-wt. of water. In a preferred embodiment, said powder contains ≤10%-wt. of water; in other words, said powder contains 10%-wt., 9%-wt., 8%-wt., 7%-wt., 6%-wt., 5%-wt., 4%-wt., 3%-wt., 2%-wt., 1%-wt., 0.5%-wt., 0.4%-wt., 0.3%-wt., 0.2%-wt., 0.1%-wt. or 0%-wt. of water. In a more preferred embodiment, said powder contains ≤7%-wt. of water; in other words, said powder contains 7%-wt., 6%-wt., 5%-wt., 4%-wt., 3%-wt., 2%-wt., 1%-wt., 0.5%-wt., 0.4%-wt., 0.3%-wt., 0.2%-wt., 0.1%-wt. or 0%-wt. of water. In a most preferred embodiment, said powder contains 5%-wt. of water; in other words, said powder contains 5%-wt., 4%-wt., 3%-wt., 2%-wt., 1%-wt., 0.5%-wt., 0.4%-wt., 0.3%-wt., 0.2%-wt., 0.1%-wt. or 0%-wt. of water.

According to another aspect, the present invention provides a dried powder, preferably a spray-dried powder, of purified saccharide solution obtained from a method as described herein, wherein said dried powder, preferably spray-dried powder, has a mean particle size of 50 to 250 μm as determined by laser diffraction. In a preferred embodiment, said dried powder, preferably spray-dried powder, has a mean particle size of 95 to 120 μm as determined by laser diffraction. In a more preferred embodiment, said dried powder, preferably spray-dried powder, has a mean particle size of 110 to 120 μm as determined by laser diffraction.

In another and/or additional preferred embodiment, the present invention provides dried powder of a purified saccharide or of a purified saccharide mixture comprising a saccharide wherein said powder when redissolved in water at a concentration of 10% (mass on volume) provides a solution with a pH between 4 and 7. In other words, said powder when redissolved in water at a concentration of 10% (mass on volume) provides a solution with a pH of 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9 or 7. In a preferred embodiment, said powder when redissolved in water at a concentration of 10% (mass on volume) provides a solution with a pH between 4 and 6, i.e. with a pH of 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9 or 6. In a more preferred embodiment, said powder when redissolved in water at a concentration of 10% (mass on volume) provides a solution with a pH between 4 and 5, i.e. with a pH of 4, 4.1, 4.2, 4.3, 4.4, 4.5, 4.6, 4.7, 4.8, 4.9 or 5. In an even more preferred embodiment, said powder when redissolved in water at a concentration of 10% (mass on volume) provides a solution with a pH between 5 and 6, i.e. with a pH of 5, 5.1, 5.2, 5.3, 5.4, 5.5, 5.6, 5.7, 5.8, 5.9 or 6.

In another and/or additional preferred embodiment, the present invention provides dried powder of a purified saccharide or of a purified saccharide mixture comprising a saccharide wherein said powder has an ash content of 10%, <9%, <8%, <7%, <6% and/or 5% on total dry solid.

In another and/or additional preferred embodiment, the present invention provides dried powder of a purified saccharide or of a purified saccharide mixture comprising a saccharide wherein said powder has an ash content of 10% on total dry solid, preferably with a lead content lower than 0.1 mg/kg dry solid, an arsenic content lower than 0.2 mg/kg dry solid, a cadmium content lower than 0.1 mg/kg dry solid and/or a mercury content lower than 0.5 mg/kg dry solid.

In another and/or additional preferred embodiment, the present invention provides dried powder of a purified saccharide or of a purified saccharide mixture comprising a saccharide wherein said powder has a lead content lower than 0.1 mg/kg dry solid, more preferably lower than 0.05 mg/kg dry solid, even more preferably below 0.02 mg/kg dry solid. In another and/or additional preferred embodiment, the present invention provides dried powder of a purified saccharide or of a purified saccharide mixture comprising a saccharide wherein said powder has an arsenic content lower than 0.2 mg/kg dry solid, more preferably lower dan 0.1 mg/kg dry solid, even more preferably lower than 0.05 mg/kg dry solid. In another and/or additional preferred embodiment, the present invention provides dried powder of a purified saccharide or of a purified saccharide mixture comprising a saccharide wherein said powder has a cadmium content lower than 0.1 mg/kg dry solid, more preferably lower than 0.05 mg/kg dry solid, even more preferably below 0.02 mg/kg dry solid. In another and/or additional preferred embodiment, the present invention provides dried powder of a purified saccharide or of a purified saccharide mixture comprising a saccharide wherein said powder has a mercury content lower than 0.5 mg/kg dry solid, more preferably lower than 0.2 mg/kg dry solid, even more preferably below 0.1 mg/kg dry solid.

In another and/or additional preferred embodiment, the present invention provides dried powder of a purified saccharide or of a purified saccharide mixture comprising a saccharide wherein said powder has a protein content below 100 mg per kg dry solid, a DNA content below 10 ng per gram dry solid and/or an endotoxin content below 10000 EU per gram dry solid. In a more preferred embodiment, the present invention provides dried powder of a purified saccharide or of a purified saccharide mixture comprising a saccharide wherein said powder has a protein content equal to or below 99 mg per kg dry solid, equal to or below 95 mg per kg dry solid, equal to or below 90 mg per kg dry solid, equal to or below 80 mg per kg dry solid, equal to or below 70 mg per kg dry solid, equal to or below 60 mg per kg dry solid, equal to or below 50 mg per kg dry solid, equal to or below 40 mg per kg dry solid, equal to or below 30 mg per kg dry solid, equal to or below 20 mg per kg dry solid or equal to or below 10 mg per kg dry solid. In another and/or additional more preferred embodiment, the present invention provides dried powder of a purified saccharide or of a purified saccharide mixture comprising a saccharide wherein said powder has a DNA content equal to or below 9 ng per gram dry solid, equal to or below 8 ng per gram dry solid, equal to or below 7 ng per gram dry solid, equal to or below 6 ng per gram dry solid, equal to or below 5 ng per gram dry solid, equal to or below 4 ng per gram dry solid, equal to or below 3 ng per gram dry solid, equal to or below 2 ng per gram dry solid, equal to or below 1 ng per gram dry solid, or no DNA. In another and/or additional more preferred embodiment, the present invention provides dried powder of a purified saccharide or of a purified saccharide mixture comprising a saccharide wherein said powder has an endotoxin content equal to or below 8000 EU per gram dry solid, equal to or below 5000 EU per gram dry solid, equal to or below 3000 EU per gram dry solid, equal to or below 1000 EU per gram dry solid, equal to or below 800 EU per gram dry solid, equal to or below 500 EU per gram dry solid, equal to or below 300 EU per gram dry solid or equal to or below 100 EU per gram dry solid.

In another and/or additional preferred embodiment, the present invention provides dried powder of a purified saccharide or of a purified saccharide mixture comprising a saccharide wherein said powder is free of DNA, recombinant DNA, proteins, proteins derived from the recombinant micro-organism and/or recombinant genetic material.

In another and/or additional preferred embodiment, the present invention provides dried powder of a purified saccharide or of a purified saccharide mixture comprising a saccharide wherein said powder has less than 125 μg/kg of 3-MCPD, wherein said 3-MCPD is the sum of 3-monochloropropanediol (3-MCPD) and 3-MCPD fatty acid esters.

In another and/or additional preferred embodiment, the present invention provides dried powder of a purified saccharide or of a purified saccharide mixture comprising a saccharide wherein said powder has less than 0.5 μg/kg epichlorohydrin.

In another and/or additional preferred embodiment, the present invention provides dried powder of a purified saccharide or of a purified saccharide mixture comprising a saccharide wherein said powder is a spray-dried powder.

In another and/or additional preferred embodiment, the present invention provides for a purified saccharide as described herein wherein any one or more of said purified saccharide is a milk oligosaccharide. In another embodiment, the present invention provides for a purified saccharide mixture comprising a saccharide as described herein wherein said purified saccharide mixture comprises a milk oligosaccharide. In a preferred embodiment, the milk oligosaccharide is a mammalian milk oligosaccharide (MMO). In a more preferred embodiment, the milk oligosaccharide is a human milk oligosaccharide (HMO). In an additional preferred embodiment, the milk oligosaccharide is selected from the group comprising 2′-fucosyllactose (2′FL), 3-fucosyllactose (3FL), 4-fucosyllactose (4FL), 6-fucosyllactose (6FL), difucosyllactose (diFL), Lacto-N-fucopentaose I (LNFP 1), Lacto-N-fucopentaose II (LNFP 11), Lacto-N-fucopentaose 11 (LNFP 111), lacto-N-fucopentaose V (LNFP V), lacto-N-fucopentaose VI (LNFP VI), lacto-N-neofucopentaose 1, lacto-N-difucohexaose I (LDFH 1), lacto-N-difucohexaose II (LDFH 11), lacto-N-triose II (LN3); lacto-N-tetraose (LNT), lacto-N-neotetraose (LNnT), lacto-N-hexaose, lacto-N-neohexaose, para-lacto-N-hexaose, para-lacto-N-neohexaose, 3′sialyllactose (3′SL), 6′sialyllactose (6′SL), sialyllacto-N-tetraose a (LSTa), sialyllacto-N-tetraose b (LSTb), sialyllacto-N-tetraose c (LSTc), sialyllacto-N-tetraose d (LSTd), disialyllacto-N-tetraose, disialyllacto-N-neotetraose, monosialyllacto-N-hexaose, disialyllacto-N-hexaose 1, disialyllacto-N-hexaose 11, monosialyllacto-N-neohexaose 1, monosialyllacto-N-neohexaose 11, disialyllacto-N-neohexaose, 3′-sialyl-3-fucosyllactose, fucodisialyllacto-N-hexaose, disialomonofucosyllacto-N-neohexaose, sialyllacto-N-fucohexaose II, disialyllacto-N-fucopentaose II and monofucosyldisialyllacto-N-tetraose and any combination thereof.

In another and/or additional preferred embodiment, the present invention provides for a purified saccharide or purified saccharide mixture as described herein, wherein the purified saccharide or purified saccharide mixture a) has a conductivity of less than 10 mS/cm at a 300 g/L solution; b) is free of recombinant DNA material, optionally free of any DNA; and/or c) is free of proteins derived from the recombinant micro-organism, optionally free of any proteins.

For identification of said saccharide as described herein, the monomeric building blocks (e.g. the monosaccharide or glycan unit composition), the anomeric configuration of side chains, the presence and location of substituent groups, degree of polymerization/molecular weight and the linkage pattern can be identified by standard methods known in the art, such as, e.g. methylation analysis, reductive cleavage, hydrolysis, GC-MS (gas chromatography-mass spectrometry), MALDI-MS (Matrix-assisted laser desorption/ionization-mass spectrometry), ESI-MS (Electrospray ionization-mass spectrometry), HPLC (High-Performance Liquid chromatography with ultraviolet or refractive index detection), HPAEC-PAD (High-Performance Anion-Exchange chromatography with Pulsed Amperometric Detection), CE (capillary electrophoresis), IR (infrared)/Raman spectroscopy, and NMR (Nuclear magnetic resonance) spectroscopy techniques. The crystal structure can be solved using, e.g., solid-state NMR, FT-IR (Fourier transform infrared spectroscopy), and WAXS (wide-angle X-ray scattering). The degree of polymerization (DP), the DP distribution, and polydispersity can be determined by, e.g., viscosimetry and SEC (SEC-HPLC, high performance size-exclusion chromatography). To identify the monomeric components of the saccharide methods such as e.g., acid-catalysed hydrolysis, HPLC (high performance liquid chromatography) or GLC (gas-liquid chromatography) (after conversion to alditol acetates) may be used. To determine the glycosidic linkages, said saccharide is methylated with methyl iodide and strong base in DMSO, hydrolysis is performed, a reduction to partially methylated alditols is achieved, an acetylation to methylated alditol acetates is performed, and the analysis is carried out by GLC/MS (gas-liquid chromatography coupled with mass spectrometry). To determine the glycan sequence, a partial depolymerization is carried out using an acid or enzymes to determine the structures. To identify the anomeric configuration, said saccharide is subjected to enzymatic analysis, e.g., it is contacted with an enzyme that is specific for a particular type of linkage, e.g., beta-galactosidase, or alpha-glucosidase, etc., and NMR may be used to analyse the products.

In another aspect, the present invention provides for a purified saccharide or purified saccharide mixture as described herein for use in medicine, preferably for use in prophylaxis or therapy of a gastrointestinal disorder.

In another aspect, the present invention provides use of a purified saccharide obtained by a method as described herein in a food or feed preparation, in a dietary supplement, in a cosmetic ingredient or in a pharmaceutical ingredient. In some embodiments, said purified saccharide is mixed with one or more ingredients suitable for food, feed, dietary supplement, pharmaceutical ingredient, cosmetic ingredient or medicine. Said purified saccharide may be used for the manufacture of a preparation, as food additive, prebiotic, symbiotic, for the supplementation of baby food, adult food, infant animal feed, adult animal feed, or as either therapeutically or pharmaceutically active compound or in cosmetic applications. In another aspect, the present invention provides use of a saccharide as described herein as additive in food, preferably as additive in human food and/or pet food, more preferably as additive in human baby food. In the context of present invention, the food is a human food, preferably infant food, human baby food and/or an infant formula or an infant supplement and the feed is a pet food, animal milk replacer, veterinary product, veterinary feed supplement, nutrition supplement, post weaning feed, or creep feed.

In another preferred embodiment, a preparation is provided that further comprises at least one probiotic microorganism. In another preferred embodiment of present invention, said preparation is a nutritional composition. In a more preferred embodiment, said preparation is a medicinal formulation, a dietary supplement, a dairy drink or an infant formula. A “prebiotic” is a substance that promotes growth of microorganisms beneficial to the host, particularly microorganisms in the gastrointestinal tract. In some embodiments, a dietary supplement provides multiple prebiotics, including said saccharide being a prebiotic purified by a method disclosed in this specification, to promote growth of one or more beneficial microorganisms. Examples of prebiotic ingredients for dietary supplements include other prebiotic molecules (such as HMOs) and plant polysaccharides (such as inulin, pectin, b-glucan and xylooligosaccharide). A “probiotic” product typically contains live microorganisms that replace or add to gastrointestinal microflora, to the benefit of the recipient. Examples of such microorganisms include Lactobacillus species (for example, L. acidophilus and L. bulgaricus), Bifidobacterium species (for example, B. animalis, B. longum and B. infantis (e.g., Bi-26)), and Saccharomyces boulardii. In some embodiments, said saccharide produced and/or purified by a method of this specification is orally administered in combination with such microorganism. Examples of further ingredients for dietary supplements include oligosaccharides (such as 2′-fucosyllactose, 3-fucosyllactose, 3′-sialyllactose, 6′-sialyllactose), disaccharides (such as lactose), monosaccharides (such as glucose, galactose, L-fucose, sialic acid, glucosamine and N-acetylglucosamine), thickeners (such as gum arabic), acidity regulators (such as trisodium citrate), water, skimmed milk, and flavourings.

In some embodiments, said saccharide purified by a method as described herein is incorporated into a human baby food (e.g., infant formula). Infant formula is generally a manufactured food for feeding to infants as a complete or partial substitute for human breast milk. In some embodiments, infant formula is sold as a powder and prepared for bottle- or cup-feeding to an infant by mixing with water. The composition of infant formula is typically designed to be roughly mimic human breast milk. In some embodiments, said saccharide purified by a method as described herein is included in infant formula to provide nutritional benefits similar to those provided by the oligosaccharides in human breast milk. In some embodiments, said purified saccharide is mixed with one or more ingredients of the infant formula. Examples of infant formula ingredients include non-fat milk, carbohydrate sources (e.g., lactose), protein sources (e.g., whey protein concentrate and casein), fat sources (e.g., vegetable oils—such as palm, high oleic safflower oil, rapeseed, coconut and/or sunflower oil; and fish oils), vitamins (such as vitamins A, Bb, Bi2, C and D), minerals (such as potassium citrate, calcium citrate, magnesium chloride, sodium chloride, sodium citrate and calcium phosphate) and possibly human milk oligosaccharides (HMOs). In some embodiments, the one or more infant formula ingredients comprise non-fat milk, a carbohydrate source, a protein source, a fat source, and/or a vitamin and mineral. In some embodiments, the one or more infant formula ingredients comprise lactose, whey protein concentrate and/or high oleic safflower oil. In some embodiments, the concentration of the oligosaccharide in the infant formula is approximately the same concentration as the concentration of the oligosaccharide generally present in human breast milk.

In some embodiments, a saccharide purified by a method as described herein is added to the infant formula with a concentration that is approximately the same concentration as the concentration of the compound generally present in human breast milk.

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry and nucleic acid chemistry and hybridization described above and below are those well-known and commonly employed in the art. Standard techniques are used for nucleic acid and peptide synthesis. Generally, purification steps are performed according to the manufacturer's specifications.

Further advantages follow from the specific embodiments and the examples. It goes without saying that the abovementioned features and the features which are still to be explained below can be used not only in the respectively specified combinations, but also in other combinations or on their own, without departing from the scope of the present invention.

Moreover, the present invention relates to the following specific embodiments:
1. A method for purification of a saccharide from a fermentation broth comprising said saccharide and biomass, the method comprising filtration of said fermentation broth on a membrane and subsequent diafiltration on said membrane, wherein said membrane comprises:
   a molecular weight cut-off ranging from 0.3 to 5 kDa, preferably from 0.5 to 4 kDa, more preferably from 1 to 3.5 kDa, even more preferably from 1.5 to 3 kDa,
   a pore size ranging from 0.001 to 0.01 μm, and/or
   a monovalent ion rejection ranging from 1 to 50%, preferably from 5 to 40%, more preferably from 10 to 20%,
under conditions permissive to produce a retentate comprising said biomass and a permeate comprising said saccharide.
2. Method according to embodiment 1, wherein said membrane is chosen from the list comprising a composite membrane, a thin-film composite membrane and a zwitterionic membrane.
3. Method according to any one of embodiment 1 or 2, wherein said membrane is based on any one of the list comprising polypiperazine-amide, polyamide, composite polyamide, composite fluoro polymer and zwitterions.
4. Method according to any one of previous embodiments, wherein said membrane is any one of flat sheet membrane or a spiral-wound membrane.
5. Method according to any one of previous embodiments, wherein said membrane is any one of an anionic, a cationic or a zwitterionic membrane.
6. Method according to any one of previous embodiments, wherein said membrane has a rejection of:
   NaCl ranging from 1 to 40%, preferably from 2 to 20%, more preferably from 5 to 10%, and/or
   MgSO₄ ranging from 10 to 98%, preferably from 20 to 97%, more preferably from 50 to 95%, even more preferably from 70 to 94%, most preferably from 70 to 90%.
7. Method according to any one of previous embodiments, wherein said membrane has not been pre-treated with a liquid that contains any one or more of alcohol, organic sulfonic acid and sulfonate, and surfactant prior to its use in said filtration and diafiltration.
8. Method according to any one of previous embodiments, wherein said filtration and/or diafiltration is/are performed at a temperature of said fermentation broth ranging from 4° C. to 55° C., preferably from 8° C. to 50° C., more preferably from 10° C. to 45° C., even more preferably from 20° C. to 40° C., even more preferably from 4° C. to 10° C.
9. Method according to any one of previous embodiments, wherein said filtration and/or diafiltration is/are performed using an inlet pressure ranging from 1 to 10 bar, preferably from 5 to 9.5 bar, more preferably from 8 to 9 bar.
10. Method according to any one of previous embodiments, wherein said diafiltration is performed at a retentate volume of at least 50%, preferably at least 60%, more preferably at least 70%, even more preferably at least 80%, even more preferably at least 90%, most preferably at least 95% of the original volume of said fermentation broth.
11. Method according to any one of previous embodiments, wherein said filtration and/or diafiltration is/are performed at a permeate flux of at least 5 L/m2/h, preferably at least 6 L/m2/h, more preferably at least 8 L/m2/h, even more preferably at least 9 L/m2/h, wherein said permeate flux is calculated as the volume in liter (L) of said permeate collected per hour (h) per square meter membrane area (m²) of said membrane.
12. Method according to any one of previous embodiments, wherein said filtration and/or diafiltration is/are performed at an initial permeate flux that is at least 10%, preferably at least 12%, more preferably at least 15%, even more preferably at least 20%, of a pure water flux, wherein:
   said initial permeate flux is calculated as the volume in liter (L) of said permeate collected per hour per square meter membrane area (m²) of said membrane of said filtration and/or diafiltration,
   said pure water flux is calculated as the volume in liter (L) of pure water collected per hour (h) per square meter membrane area (m²) of said membrane of said filtration and/or diafiltration, and
   said initial permeate flux and said pure water flux are measured in a filtration and/or diafiltration on the same membrane under identical conditions of temperature and pressure from the start of said filtration and/or diafiltration and for at least 10 minutes, preferably for at least 20 minutes, more preferably for at least 30 minutes, even more preferably for at least 1 hour.
13. Method according to any one of previous embodiments, wherein said filtration and/or diafiltration is/are performed under conditions such that:
   the permeate flux in said filtration and/or diafiltration does not decline, or
   the decline in permeate flux in said filtration and/or diafiltration ranges from 0 to 15%, preferably from 0 to 10%, more preferably from 0 to 5%, even more preferably from 0.5 to 2.5%, even more preferably from 0.1 to 1%.
14. Method according to any one of previous embodiments, wherein said filtration and/or diafiltration is/are performed under conditions such that at the end of said filtration and/or diafiltration the surface area of said membrane is covered for 0 to 10%, preferably for 0.01 to 5%, more preferably for 0.1 to 1%, with a fouling cake.
15. Method according to any one of previous embodiments, wherein said diafiltration is performed until the original constant retentate volume has been collected at least one time, preferably at least two times, more preferably at least 3 times, even more preferably at least 4 times, most preferably at least 5 times.
16. Method according to any one of previous embodiments, wherein said diafiltration is performed until a conductivity is reached for the retentate of 40 mS/cm, preferably 15 mS/cm, 10 mS/cm, 5 mS/cm, 1 mS/cm, 0.1 mS/cm, 0.01 mS/cm, <0.001 mS/cm.
17. Method according to any one of previous embodiments, wherein said membrane is used only once under said conditions in said method.
18. Method according to any one of previous embodiments, wherein the temperature of said fermentation broth is adjusted to a temperature of from 0° C. to 130° C., preferably from 2° C. to 122° C., more preferably from 4° C. to 80° C., even more preferably from 8° C. to 60° C., even more preferably from 10° C. to 55° C., even more preferably 20° C. to 45° C., even more preferably from 21° C. to 40° C., even more preferably from 22° C. to 37° C., even more preferably from 25° C. to 30° C.
19. Method according to any one of previous embodiments, wherein said fermentation broth is heat treated to a temperature ranging from 60° C. to 130° C., preferably ranging from 80° C. to 122° C., prior to said filtration.
20. Method according to any one of previous embodiments, wherein the temperature of said fermentation broth is adjusted to a temperature of from 36° C. to 65° C., wherein said temperature is within 5° C. of a temperature at which the fermentation broth exhibits maximum turbidity, preferably from 36° C. to 60° C., more preferably from 40° C. to 55° C., even more preferably from 40° C. to 45° C.
21. Method according to any one of previous embodiments, wherein the pH of said fermentation broth has not been adjusted prior to said filtration.
22. Method according to any one of previous embodiments, wherein the pH of said fermentation broth ranges from 2 to 7, preferably from 3 to 6.8, more preferably from 4 to 6.5, even more preferably from 5 to 6.
23. Method according to any one of previous embodiments, wherein said biomass in said fermentation broth has a cell dry weight (CDW) that is at least 30 g/L, preferably at least 40 g/L, more preferably at least 50 g/L, even more preferably at least 60 g/L, even more preferably at least 70 g/L, even more preferably at least 80 g/L, even more preferably at least 90 g/L, most preferably at least 100 g/L.
24. Method according to any one of previous embodiments, wherein said fermentation broth is not subjected to dilution prior to said filtration.
25. Method according to any one of previous embodiments, wherein said saccharide is chosen from the list comprising monosaccharide; disaccharide; oligosaccharide; polysaccharide; neutral (non-charged) saccharide; negatively charged, preferably sialylated, saccharide; milk oligosaccharide, preferably a mammalian milk oligosaccharide (MMO), more preferably a human milk oligosaccharide (HMO);
lactose; sucrose; glucose; glycerol; galactose; fucose; mannose; N-acetylglucosamine; N-acetylgalactosamine; lactosamine; lacto-N-biose; O-antigen; enterobacterial common antigen (ECA); the oligosaccharide repeats present in capsular polysaccharides; peptidoglycan; an amino-sugar; Lewis-type antigen oligosaccharide; an antigen of the human ABO blood group system; an animal oligosaccharide, preferably selected from the group consisting of N-glycans and O-glycans; a plant oligosaccharide, preferably selected from the group consisting of N-glycans and O-glycans; fucosylated oligosaccharide, preferably selected from the group comprising 2′-fucosyllactose (2′FL), 3-fucosyllactose (3FL), 4-fucosyllactose (4FL), 6-fucosyllactose (6FL), 2′,3-difucosyllactose (diFL), lacto-N-fucopentaose I, lacto-N-neofucopentaose I, lacto-N-fucopentaose II, lacto-N-fucopentaose III, lacto-N-fucopentaose V, lacto-N-fucopentaose VI, lacto-N-neofucopentaose V, lacto-N-difucohexaose I, lacto-N-difucohexaose II, difucosyl-lacto-N-hexaose and difucosyl-lacto-N-neohexaose; sialylated oligosaccharide, preferably selected from the group comprising 3′sialyllactose (3′SL), 6′sialyllactose (6′SL), sialyllacto-N-tetraose a (LSTa), sialyllacto-N-tetraose b (LSTb), sialyllacto-N-tetraose c (LSTc), sialyllacto-N-tetraose d (LSTd), disialyllacto-N-tetraose, disialyllacto-N-neotetraose, monosialyllacto-N-hexaose, disialyllacto-N-hexaose I, disialyllacto-N-hexaose II, monosialyllacto-N-neohexaose I, monosialyllacto-N-neohexaose II, disialyllacto-N-neohexaose, 3′-sialyl-3-fucosyllactose, fucodisialyllacto-N-hexaose, disialomonofucosyllacto-N-neohexaose, sialyllacto-N-fucohexaose II, disialyllacto-N-fucopentaose II and monofucosyldisialyllacto-N-tetraose; N-acetylglucosamine containing neutral (non-charged) oligosaccharide, preferably selected from the group comprising lacto-N-triose II (LN3), lacto-N-tetraose (LNT), lacto-N-neotetraose (LNnT), 6′-galactosyllactose, 3′-galactosyllactose, lacto-N-hexaose, lacto-N-neohexaose, para-lacto-N-hexaose, para-lacto-N-neohexaose; N-acetyllactosamine containing oligosaccharide; lacto-N-biose containing oligosaccharide; non-fucosylated neutral (non-charged) oligosaccharide; chitosan; chitosan comprising oligosaccharide; heparosan; chondroitin sulphate; glycosaminoglycan oligosaccharide; heparin; heparan sulphate; dermatan sulphate; hyaluronan; hyaluronic acid; and keratan sulphate.
26. Method according to any one of previous embodiments, wherein said fermentation broth originates from a fermentation of a cell producing said saccharide.
27. Method according to embodiment 26, wherein said cell has been metabolically engineered to produce said saccharide.
28. Method according to any one of previous embodiments, wherein said saccharide is accompanied in said fermentation broth by sialic acid, ashes, one or more monosaccharide(s), one or more activated monosaccharide(s), one or more phosphorylated monosaccharide(s) and/or one or more other saccharide(s), preferably, said ashes comprise sulphates and phosphates.
29. Method according to any one of embodiments 26 to 28, wherein said cell has been metabolically engineered to produce said saccharide and one or more of i) sialic acid, ii) one or more monosaccharide(s), iii) one or more activated monosaccharide(s), iv) one or more phosphorylated monosaccharide(s), v) one or more other saccharide(s).
30. Method according to any one of embodiments 26 to 29, wherein said cell produces said saccharide from one or more internalized precursor(s).
31. Method according to any one of embodiments 26 to 30, wherein said cell is a prokaryotic cell, preferably selected from the group consisting of yeast cells, bacterial cells, archaebacterial cells and fungal cells.
32. Method according to any one of embodiments 26 to 31, wherein said cell is a bacterium, fungus, yeast or a protozoan cell,
   preferably, said bacterium belongs to a phylum chosen from the group comprising Proteobacteria, Firmicutes, Cyanobacteria, Deinococcus-Thermus and Actinobacteria; more preferably, said bacterium belongs to a family chosen from the group comprising Enterobacteriaceae, Bacillaceae, Lactobacillaceae, Corynebacteriaceae and Vibrionaceae; even more preferably, said bacterium is chosen from the list comprising an Escherichia coli strain, a Bacillus subtilis strain, a Vibrio natriegens strain; even more preferably said Escherichia coli strain is a K-12 strain, most preferably said Escherichia coli K-12 strain is E. coli MG1655,
   preferably, said fungus belongs to a genus chosen from the group comprising Rhizopus, Dictyostelium, Penicillium, Mucor or Aspergillus,
   preferably, said yeast belongs to a genus chosen from the group comprising Saccharomyces, Zygosaccharomyces, Pichia, Komagataella, Hansenula, Yarrowia, Starmerella, Kluyveromyces, Debaromyces, Candida, Schizosaccharomyces, Schwanniomyces or Torulaspora; more preferably, said yeast is selected from the group consisting of: Saccharomyces cerevisiae, Hansenula polymorpha, Kluyveromyces lactis, Kluyveromyces marxianus, Pichia pastoris, Pichia methanolica, Pichia stipites, Candida boidinii, Schizosaccharomyces pombe, Schwanniomyces occidentalis, Torulaspora delbrueckii, Yarrowia lipolytica, Zygosaccharomyces rouxii, and Zygosaccharomyces bailii,
   preferably, said protozoan cell is a Leishmania tarentolae cell.
33. Method according to any one of embodiments 26 to 32, wherein said cell is an E. coli or yeast with a lactose permease positive phenotype, preferably wherein said lactose permease is coded by the gene LacY or LAC12, respectively.
34. Method according to any one of embodiments 26 to 33, wherein said cell is cultivated in culture medium comprising a carbon source comprising a monosaccharide, disaccharide, oligosaccharide, polysaccharide, polyol, glycerol, a complex medium including molasses, corn steep liquor, peptone, tryptone or yeast extract; preferably, said carbon source is chosen from the list comprising glucose, N-acetylglucosamine (GlcNAc), glycerol, fructose, sucrose, maltose, lactose, arabinose, malto-oligosaccharides, maltotriose, sorbitol, xylose, rhamnose, galactose, mannose, methanol, ethanol, trehalose, starch, cellulose, hemi-cellulose, molasses, corn-steep liquor, high-fructose syrup, acetate, citrate, lactate and pyruvate; preferably, said culture medium is a chemically defined medium.
35. Method according to embodiment 34, wherein said culture medium is a minimal salt medium comprising sulphate, phosphate, chloride, ammonium, calcium, magnesium, sodium, potassium, iron, copper, zinc, manganese, cobalt, and/or selenium.
36. Method according to any one of previous embodiments, wherein said fermentation broth further comprises antifoam and/or proteins.
37. Method according to any one of previous embodiments, wherein said fermentation broth further comprises at least 0.01% antifoam (v/v), preferably at least 0.1% antifoam (v/v), more preferably at least 1% antifoam (v/v).
38. Method according to any one of embodiment 36 or 37, wherein said antifoam and/or proteins is/are present in said retentate.
39. Method according to any one of previous embodiments, wherein the purity of said saccharide in said fermentation broth is <70%, <60%, <50%, <40%, <30%, <20%, <10% on total dry solid before purification by said method.
40. Method according to any one of previous embodiments, wherein said method further comprises any one or more of concentration, homogenization, clarification, clearing, centrifugation, decantation, dilution, pH adjustment, temperature adjustment, filtration, ultrafiltration, microfiltration, diafiltration, reverse osmosis, electrodialysis, electrodeionization, nanofiltration, dialysis, use of activated charcoal or carbon, use of solvents, use of alcohols, use of aqueous alcohol mixtures, use of charcoal, tangential flow high-performance filtration, tangential flow ultrafiltration, affinity chromatography, ion exchange, ion exchange chromatography, mixed bed ion exchange, hydrophobic interaction chromatography, gel filtration, ligand exchange chromatography, column chromatography, cation exchange adsorbent resin, anion exchange adsorbent resin, use of an adsorbent material, use of ion exchange resin, evaporation, wiped film evaporation, falling film evaporation, pasteurization, enzymatic treatment, decolorization and drying, in any order.
41. Method according to embodiment 40, wherein said method comprises enzymatic treatment comprising incubation with one or more enzymes selected from the group comprising glycosidase, lactase, β-galactosidase, fucosidase, sialidase, maltase, amylase, hexaminidase, glucuronidase, trehalase, and invertase.
42. Method according to any one of embodiment 40 or 41, wherein said method comprises enzymatic treatment converting lactose, sucrose, malto-oligosaccharides, maltotriose, sorbitol, trehalose, starch, cellulose, hemi-cellulose, lignocellulose, molasses, corn-steep liquor and/or high-fructose syrup to monosaccharides.
43. Method according to any one of embodiments 40 to 42, wherein said method comprises drying chosen from the list comprising spray drying, lyophilization, spray freeze drying, freeze spray drying, band drying, belt drying, vacuum band drying, vacuum belt drying, drum drying, roller drying, vacuum drum drying, vacuum roller drying, and agitated thin film drying.
44. Method according to any one of previous embodiments, wherein the purity of said saccharide obtained in a purified saccharide solution at the end of said method is at least 70%, at least 75%, at least 80%, at least 85%, at least 90%, at least 95%, at least 97%, at least 98%, at least 99% on total dry solid.
45. Method according to any one of previous embodiments, wherein the yield of purification of the saccharide obtained in the purified saccharide solution at the end of said method is at least 60%, at least 65%, at least 70%, at least 75%, at least 80%, at least 85%, at least 90%, at least 95%, at least 97%, at least 98%, at least 99%.
46. Method according to any one of previous embodiments, wherein the purified saccharide solution obtained at the end of said method has an ash content of 10% on total dry solid, preferably 9% on total dry solid, more preferably 8% on total dry solid, even more preferably 7% on total dry solid, even more preferably 6% on total dry solid, even more preferably 5% on total dry solid.
47. Method according to any one of previous embodiments, wherein the purified saccharide solution obtained at the end of said method has an ash content of 10% on total dry solid, preferably with a lead content lower than 0.1 mg/kg dry solid, an arsenic content lower than 0.2 mg/kg dry solid, a cadmium content lower than 0.1 mg/kg dry solid and/or a mercury content lower than 0.5 mg/kg dry solid.
48. Method according to any one of previous embodiments, wherein the purified saccharide solution obtained at the end of said method is filter-sterilized and/or subjected to endotoxin removal, preferably by filtration through a 3 kDa filter.
49. Method according to any one of previous embodiments, wherein the purified saccharide solution obtained at the end of said method has a protein content below 100 mg per kg dry solid, a DNA content below 10 ng per gram dry solid and/or an endotoxin content below 10000 EU per gram dry solid, preferably the purified saccharide solution is free of DNA, proteins, and/or recombinant genetic material.
50. Method according to any one of previous embodiments, wherein the purified saccharide solution obtained at the end of said method is further i) concentrated to a syrup of at least 20% dry matter, preferably at least 30% dry matter, more preferably at least 40% dry matter; ii) crystallised; iii) dried to a powder or iv) granulated.
51. Method according to any one of previous embodiments, wherein the purified saccharide solution obtained at the end of said method is further concentrated to a saccharide concentration of >100 g/L, preferably >200 g/L, more preferably >300 g/L, more preferably >400 g/L, more preferably >500 g/L, more preferably >600 g/L, most preferably between 300 g/L and 650 g/L and/or at a temperature of <80° C., preferably <60° C., more preferably <50° C., more preferably 20° C. to 50° C., even more preferably 30° C. to 45° C., preferably concentrated by a method comprising using vacuum evaporation or reverse osmosis or nanofiltration.
52. Method according to any one of previous embodiments, wherein the purified saccharide solution obtained at the end of said method comprises a saccharide which is concentrated to a concentration of >1.5 M and cooled to a temperature <25° C., more preferably <8° C., to obtain crystalline material of the saccharide.
53. Method according to any one of embodiments 1 to 51, wherein the purified saccharide solution obtained at the end of said method is dried by any one or more of drying steps chosen from the list comprising spray drying, lyophilization, evaporation, precipitation, spray freeze drying, freeze spray drying, band drying, belt drying, vacuum band drying, vacuum belt drying, drum drying, roller drying, vacuum drum drying, vacuum roller drying and agitated thin film drying.
54. Method according to embodiment 53, wherein the purified saccharide solution obtained at the end of said method is dried by spray-drying, freeze-drying or agitated thin film drying and preferably wherein the pH of said purified saccharide solution is ranging from 2 to 7, preferably from 3 to 6, more preferably from 4 to 5.
55. Method according to any one of embodiment 53 or 54, wherein the purified saccharide solution obtained at the end of said method is dried by spray-drying, preferably particularly spray-dried at a saccharide solution concentration of 20-60 (w/v), preferably 30-50 (w/v), more preferably 35-45 (w/v), with a nozzle temperature of 110-150° C., preferably 120-140° C., more preferably 125-135° C. and/or an outlet temperature of 60-80° C., preferably 65-70° C.
56. The purified saccharide solution, the purified saccharide or the purified saccharide mixture comprising a saccharide obtainable, preferably obtained, by a method according to any one of previous embodiments.
57. Purified saccharide obtainable, preferably obtained, by a method according to any one of embodiments 1 to 55, wherein the purified saccharide solution comprising said purified saccharide is i) dried, preferably spray-dried or dried via an agitated thin film dryer; ii) lyophilized; iii) crystallized or iv) concentrated to a syrup of at least 20% dry matter, preferably at least 30% dry matter, more preferably at least 40% dry matter.
58. Purified saccharide mixture comprising a saccharide obtainable, preferably obtained, by a method according to any one of embodiments 1 to 55, wherein the purified saccharide solution comprising said purified saccharide mixture comprising a saccharide is i) dried, preferably spray-dried or dried via an agitated thin film dryer; ii) lyophilised; iii) crystallized or iv) concentrated to a syrup of at least 20% dry matter, preferably at least 30% dry matter, more preferably at least 40% dry matter.
59. Saccharide purified according to the method according to any one of embodiments 1 to 55 and wherein the purified saccharide obtained after said method contains less than 10% ash.
60. Spray-dried saccharide or saccharide mixture comprising a saccharide, wherein said saccharide or saccharide mixture is purified according to the method according to any one of embodiments 1 to 55 and wherein said spray-dried saccharide or saccharide mixture obtained after said method contains less than 10% ash.
61. Dried powder of purified saccharide solution obtained from a method according to any one of embodiments 53 to 55, wherein said dried powder:
   contains <15%-wt. of water, preferably <10%-wt. of water, more preferably <7%-wt. of water, most preferably 5%-wt. of water, and/or
   has a mean particle size of 50 to 250 μm, preferably of 95 to 120 μm, more preferably of 110 to 120 μm, wherein said particle size is determined by laser diffraction,
preferably said powder is a spray-dried powder.
62. Dried powder according to any one of embodiments 57, 58, 60, 61, wherein said powder when redissolved in water at a concentration of 10% (mass on volume) provides a solution with a pH between 4 and 7, preferably with a pH between 4 and 6, more preferably with a pH between 4 and 5, even more preferably with a pH between 5 and 6.
63. Purified saccharide according to any one of embodiments 56, 57, 59 to 62, wherein any one or more of said saccharide is a milk oligosaccharide, preferably a mammalian milk oligosaccharide (MMO), more preferably a human milk oligosaccharide (HMO).
64. Purified saccharide mixture comprising a saccharide according to any one of embodiments 56, 58, 60 to 62, wherein said purified saccharide mixture comprises a milk oligosaccharide, preferably a mammalian milk oligosaccharide (MMO), more preferably a human milk oligosaccharide (HMO).
65. Purified saccharide or purified saccharide mixture according to any one of embodiments 56 to 64, wherein the purified saccharide or purified saccharide mixture a) has a conductivity of less than 10 mS/cm at a 300 g/L solution; b) is free of recombinant DNA material, optionally free of any DNA; and/or c) is free of proteins derived from the recombinant micro-organism, optionally free of any proteins.
66. Purified saccharide or purified saccharide mixture according to any one of embodiments 56 to 65 for use in medicine, preferably for use in prophylaxis or therapy of a gastrointestinal disorder.
67. Use of a purified saccharide obtained by a method of any one of embodiments 1 to 55 in a food or feed preparation, in a dietary supplement, in a cosmetic ingredient or in a pharmaceutical ingredient.
68. Use of a purified saccharide according to any one of embodiments 56 to 65 in a food or feed preparation, in a dietary supplement, in a cosmetic ingredient or in a pharmaceutical ingredient.
69. Use according to any one of embodiment 67 or 68, wherein:
   the food is a human food, preferably infant food, human baby food and/or an infant formula or an infant supplement, and/or
   the feed is a pet food, animal milk replacer, veterinary product, veterinary feed supplement, nutrition supplement, post weaning feed, or creep feed.
70. Use of a milk oligosaccharide according to any one of embodiment 63 or 64 as additive in food, preferably as additive in human food and/or pet food, more preferably as additive in human baby food.

More specifically, the present invention relates to the following preferred specific embodiments:
1. A method for purification of a saccharide from a fermentation broth comprising said saccharide and biomass, wherein said fermentation broth originates from a fermentation of a cell producing said saccharide and wherein said biomass consists essentially of or consists of intact cells, disrupted cells, cell fragments, cell walls, phospholipids, cell membranes, proteins, protein fragments, polysaccharides, polynucleotides and large organic compounds produced by the cell of said fermentation, the method comprising filtration of said fermentation broth on a membrane and subsequent diafiltration on said membrane, wherein said membrane comprises:
   a molecular weight cut-off ranging from 0.3 to 5 kDa, from 0.5 to 4 kDa, from 1 to 3.5 kDa and/or from 1.5 to 3 kDa,
   a pore size ranging from 0.001 to 0.01 μm, and/or
   a monovalent ion rejection ranging from 1 to 50%, from 5 to 40% and/or from 10 to 20%, under conditions permissive to collect (1) a retentate comprising, consisting of or consisting essentially of said biomass and (2) a permeate comprising essentially all or all of said saccharide.
2. Method according to preferred embodiment 1, wherein said membrane:
   is selected from the list comprising a composite membrane, a thin-film composite membrane and a zwitterionic membrane,
   is based on any one of the list comprising polypiperazine-amide, polyamide, composite polyamide, composite fluoro polymer and zwitterions,
   is any one of flat sheet membrane or a spiral-wound membrane,
   is any one of an anionic, a cationic or a zwitterionic membrane,
   has a rejection of NaCl ranging from 1 to 40%, from 2 to 20% and/or from 5 to 10%,
   has a rejection of MgSO₄ ranging from 10 to 98%, 20 to 97%, from 50 to 95%, from 70 to 94% and/or from 70 to 90%, and/or
   has not been pre-treated with a liquid that contains any one or more of alcohol, organic sulfonic acid and sulfonate, and surfactant prior to its use in said filtration and diafiltration.
3. Method according to any one of preferred embodiment 1 or 2, wherein said filtration and/or diafiltration is/are performed:
   at a temperature of said fermentation broth ranging from 4° C. to 55° C., from 8° C. to 50° C., from 10° C. to 45° C., from 20° C. to 40° C. and/or from 4° C. to 10° C.,
   using an inlet pressure ranging from 1 to 10 bar, from 5 to 9.5 bar and/or from 8 to 9 bar,
   at a permeate flux of at least 5 L/m2/h, at least 6 L/m2/h, at least 8 L/m2/h and/or at least 9 L/m2/h, wherein said permeate flux is calculated as the volume in liter (L) of said permeate collected per hour (h) per square meter membrane area (m²) of said membrane,
   at an initial permeate flux that is at least 10%, preferably at least 12%, more preferably at least 15%, even more preferably at least 20%, of a pure water flux, wherein:
      said initial permeate flux is calculated as the volume in liter (L) of said permeate collected per hour per square meter membrane area (m²) of said membrane of said filtration and/or diafiltration,
      said pure water flux is calculated as the volume in liter (L) of pure water collected per hour (h) per square meter membrane area (m²) of said membrane of said filtration and/or diafiltration, and
      said initial permeate flux and said pure water flux are measured in a filtration and/or diafiltration on the same membrane under identical conditions of temperature and pressure from the start of said filtration and/or diafiltration and for at least 10 minutes, for at least 20 minutes, for at least 30 minutes and/or for at least 1 hour.
4. Method according to any one of previous preferred embodiments, wherein said filtration and/or diafiltration is/are performed under conditions such that:
   the decline in permeate flux in said filtration and/or diafiltration ranges from 0.1 to 15%, from 0.1 to 10%, from 0.1 to 5%, from 0.5 to 2.5% and/or from 0.1 to 1%,
   said diafiltration is performed until a conductivity is reached for the retentate of 40 mS/cm, 15 mS/cm, <10 mS/cm, 5 mS/cm, 1 mS/cm, 0.1 mS/cm, 0.01 mS/cm and/or 0.001 mS/cm,
   said diafiltration is performed at a retentate volume of at least 50%, at least 60%, at least 70%, at least 80%, at least 90% and/or at least 95% of the original volume of said fermentation, and/or
   at the end of said filtration and/or diafiltration the surface area of said membrane is covered for 0 to 10%, for 0.01 to 5% and/or for 0.1 to 1%, with a fouling cake, wherein said filtration is performed until a retentate volume is obtained of at least 50%, at least 60%, at least 70%, at least 80%, at least 90% and/or at least 95% of the original volume of said fermentation,
wherein said diafiltration is performed until the original retentate volume has been collected at least one time, at least two times, at least 3 times, at least 4 times and/or at least 5 times.
5. Method according to any one of previous preferred embodiments, wherein:
   the temperature of said fermentation broth is adjusted to a temperature of from 0° C. to 130° C., from 2° C. to 122° C., from 4° C. to 80° C., from 8° C. to 60° C., from 10° C. to 55° C., from 20° C. to 45° C., from 21° C. to 40° C., from 22° C. to 37° C. and/or from 25° C. to 30° C.,
   the temperature of said fermentation broth is adjusted to a temperature of from 36° C. to 65° C., from 36° C. to 60° C., from 40° C. to 55° C. and/or from 40° C. to 45° C. wherein said temperature is within 5° C. of a temperature at which the fermentation broth exhibits maximum turbidity, and/or
   said fermentation broth is heat treated to a temperature ranging from 60° C. to 130° C. and/or from 80° C. to 122° C. prior to said filtration.
6. Method according to any one of previous preferred embodiments, wherein the pH of said fermentation broth:
   has not been adjusted prior to said filtration, and/or
   ranges from 2 to 7, from 3 to 6.8, from 4 to 6.5 and/or from 5 to 6.
7. Method according to any one of previous preferred embodiments, wherein said biomass in said fermentation broth has a cell dry weight (CDW) that is at least 30 g/L, at least 40 g/L, at least 50 g/L, at least 60 g/L, at least 70 g/L, at least 80 g/L, at least 90 g/L and/or at least 100 g/L.
8. Method according to any one of previous preferred embodiments, wherein said fermentation broth is not subjected to dilution prior to said filtration.
9. Method according to any one of previous preferred embodiments, wherein said saccharide is selected from the list comprising monosaccharide; disaccharide; oligosaccharide; polysaccharide; neutral (non-charged) saccharide; negatively charged saccharide; sialylated saccharide; milk oligosaccharide; a mammalian milk oligosaccharide (MMO); a human milk oligosaccharide (HMO); lactose; sucrose; glucose; glycerol; galactose; fucose; mannose; N-acetylglucosamine; N-acetylgalactosamine; lactosamine; lacto-N-biose; O-antigen; enterobacterial common antigen (ECA); the oligosaccharide repeats present in capsular polysaccharides; peptidoglycan; an amino-sugar; Lewis-type antigen oligosaccharide; an antigen of the human ABO blood group system; an animal oligosaccharide; an animal oligosaccharide selected from the list consisting of N-glycans and O-glycans; a plant oligosaccharide; a plant oligosaccharide selected from the list consisting of N-glycans and O-glycans; fucosylated oligosaccharide; fucosylated oligosaccharide selected from the list comprising 2′-fucosyllactose (2′FL), 3-fucosyllactose (3FL), 4-fucosyllactose (4FL), 6-fucosyllactose (6FL), 2′,3-difucosyllactose (diFL), lacto-N-fucopentaose I, lacto-N-neofucopentaose I, lacto-N-fucopentaose II, lacto-N-fucopentaose III, lacto-N-fucopentaose V, lacto-N-fucopentaose VI, lacto-N-neofucopentaose V, lacto-N-difucohexaose I, lacto-N-difucohexaose II, difucosyl-lacto-N-hexaose and difucosyl-lacto-N-neohexaose; sialylated oligosaccharide; sialylated oligosaccharide selected from the list comprising 3′sialyllactose (3′SL), 6′sialyllactose (6′SL), sialyllacto-N-tetraose a (LSTa), sialyllacto-N-tetraose b (LSTb), sialyllacto-N-tetraose c (LSTc), sialyllacto-N-tetraose d (LSTd), disialyllacto-N-tetraose, disialyllacto-N-neotetraose, monosialyllacto-N-hexaose, disialyllacto-N-hexaose I, disialyllacto-N-hexaose II, monosialyllacto-N-neohexaose I, monosialyllacto-N-neohexaose II, disialyllacto-N-neohexaose, 3′-sialyl-3-fucosyllactose, fucodisialyllacto-N-hexaose, disialomonofucosyllacto-N-neohexaose, sialyllacto-N-fucohexaose II, disialyllacto-N-fucopentaose II and monofucosyldisialyllacto-N-tetraose; N-acetylglucosamine containing neutral (non-charged) oligosaccharide; N-acetylglucosamine containing neutral (non-charged) oligosaccharide selected from the list comprising lacto-N-triose II (LN3), lacto-N-tetraose (LNT), lacto-N-neotetraose (LNnT), 6′-galactosyllactose, 3′-galactosyllactose, lacto-N-hexaose, lacto-N-neohexaose, para-lacto-N-hexaose, para-lacto-N-neohexaose; N-acetyllactosamine containing oligosaccharide; lacto-N-biose containing oligosaccharide; non-fucosylated neutral (non-charged) oligosaccharide; chitosan; chitosan comprising oligosaccharide; heparosan; chondroitin sulphate; glycosaminoglycan oligosaccharide; heparin; heparan sulphate; dermatan sulphate; hyaluronan; hyaluronic acid; and keratan sulphate.
10. Method according to any one of previous preferred embodiments, wherein said cell has been metabolically engineered to produce said saccharide.
11. Method according to any one of previous preferred embodiments, wherein said saccharide is accompanied in said fermentation broth by sialic acid, ashes, ashes comprising sulphates and phosphates, one or more monosaccharide(s), one or more activated monosaccharide(s), one or more phosphorylated monosaccharide(s) and/or one or more other saccharide(s).
12. Method according to any one of previous preferred embodiments, wherein said cell is a prokaryotic cell, yeast cell, bacterial cell, archaebacterial cell or fungal cell.
13. Method according to any one of previous preferred embodiments, wherein said cell is cultivated in:
   culture medium comprising a carbon source comprising a monosaccharide, disaccharide, oligosaccharide, polysaccharide, polyol, glycerol, a complex medium including molasses, corn steep liquor, peptone, tryptone or yeast extract;
   culture medium comprising a carbon source wherein said carbon source is selected from the list comprising glucose, N-acetylglucosamine (GlcNAc), glycerol, fructose, sucrose, maltose, lactose, arabinose, malto-oligosaccharides, maltotriose, sorbitol, xylose, rhamnose, galactose, mannose, methanol, ethanol, trehalose, starch, cellulose, hemi-cellulose, molasses, corn-steep liquor, high-fructose syrup, acetate, citrate, lactate and pyruvate;
   a chemically defined medium, and/or
   a minimal salt medium comprising sulphate, phosphate, chloride, ammonium, calcium, magnesium, sodium, potassium, iron, copper, zinc, manganese, cobalt, and/or selenium.
14. Method according to any one of previous preferred embodiments, wherein said fermentation broth further comprises:
   antifoam and/or proteins,
   at least 0.01% antifoam (v/v), at least 0.1% antifoam (v/v) and/or at least 1% antifoam (v/v), and/or
   antifoam and/or proteins wherein said antifoam and/or proteins is/are present in said retentate.
15. Method according to any one of previous preferred embodiments, wherein:
   the purity of said saccharide in said fermentation broth is <70%, <60%, <50%, <40%, <30%, <20%, <10% on total dry solid before purification by said method,
   the purity of said saccharide obtained in a purified saccharide solution at the end of said method is at least 70%, at least 75%, at least 80%, at least 85%, at least 90%, at least 95%, at least 97%, at least 98%, at least 99% on total dry solid, and/or
   the yield of purification of the saccharide obtained in the purified saccharide solution at the end of said method is at least 60%, at least 65%, at least 70%, at least 75%, at least 80%, at least 85%, at least 90%, at least 95%, at least 97%, at least 98%, at least 99%.
16. Method according to any one of previous preferred embodiments, wherein said method further comprises any one or more of concentration; homogenization; clarification; clearing; centrifugation; decantation; dilution; pH adjustment; temperature adjustment; filtration; ultrafiltration; microfiltration; diafiltration; reverse osmosis; electrodialysis; electrodeionization; nanofiltration; dialysis; use of activated charcoal or carbon; use of solvents; use of alcohols; use of aqueous alcohol mixtures; use of charcoal; tangential flow high-performance filtration; tangential flow ultrafiltration; affinity chromatography; ion exchange; ion exchange chromatography; mixed bed ion exchange; hydrophobic interaction chromatography; gel filtration; ligand exchange chromatography; column chromatography; cation exchange adsorbent resin; anion exchange adsorbent resin; use of an adsorbent material; use of ion exchange resin; evaporation; vacuum evaporation; wiped film evaporation; falling film evaporation; pasteurization; enzymatic treatment; enzymatic treatment comprising incubation with one or more enzymes selected from the group comprising glycosidase, lactase, β-galactosidase, fucosidase, sialidase, maltase, amylase, hexaminidase, glucuronidase, trehalase, and invertase; enzymatic treatment converting lactose, sucrose, malto-oligosaccharides, maltotriose, sorbitol, trehalose, starch, cellulose, hemi-cellulose, lignocellulose, molasses, corn-steep liquor and/or high-fructose syrup to monosaccharides; decolorization; drying; drying selected from the list comprising spray drying, lyophilization, spray freeze drying, freeze spray drying, band drying, belt drying, vacuum band drying, vacuum belt drying, drum drying, roller drying, vacuum drum drying, vacuum roller drying, and agitated thin film drying; in any order.
17. Method according to any one of previous preferred embodiments, wherein the purified saccharide solution obtained at the end of said method:
   has an ash content of ≤10%, ≤9%, ≤8%, ≤7%, ≤6% and/or ≤5% on total dry solid,
   has an ash content of ≤10% on total dry solid with a lead content lower than 0.1 mg/kg dry solid,
   has an ash content of ≤10% on total dry solid with an arsenic content lower than 0.2 mg/kg dry solid,
   has an ash content of ≤10% on total dry solid with a cadmium content lower than 0.1 mg/kg dry solid,
   has an ash content of ≤10% on total dry solid with a mercury content lower than 0.5 mg/kg dry solid,
   has a protein content below 100 mg per kg dry solid, a DNA content below 10 ng per gram dry solid and/or an endotoxin content below 10000 EU per gram dry solid,
   is free of DNA, recombinant DNA, proteins, proteins derived from the recombinant micro-organism and/or recombinant genetic material,
   has less than 125 μg/kg of 3-MCPD, wherein said 3-MCPD is the sum of 3-monochloropropanediol (3-MCPD) and 3-MCPD fatty acid esters,
   has less than 0.5 μg/kg epichlorohydrin,
   is filter-sterilized, subjected to endotoxin removal and/or subjected to endotoxin removal by filtration through a 3 kDa filter,
   has a conductivity of less than 10 mS/cm at a 300 g/L solution,
   is further i) concentrated to a syrup of at least 20% dry matter, at least 30% dry matter and/or at least 40% dry matter; ii) crystallised; iii) dried to a powder or iv) granulated,
   is further concentrated to a saccharide concentration of >100 g/L, >200 g/L, >300 g/L, >400 g/L, >500 g/L, >600 g/L and/or between 300 g/L and 650 g/L, and/or at a temperature of <80° C., <60° C., <50° C., between 20° C. to 50° C., and/or 30° C. to 45° C.,
   is further concentrated by a method comprising using vacuum evaporation or reverse osmosis or nanofiltration,
   is dried by any one or more of drying steps selected from the list comprising spray drying, lyophilization, evaporation, precipitation, freeze drying, spray freeze drying, freeze spray drying, band drying, belt drying, vacuum band drying, vacuum belt drying, drum drying, roller drying, vacuum drum drying, vacuum roller drying and agitated thin film drying,
   comprises a saccharide which is concentrated to a concentration of >1.5 M and cooled to a temperature <25° C. and/or <8° C., to obtain crystalline material of the saccharide,
   is spray-dried at a saccharide solution concentration of 20-60 (w/v), 30-50 (w/v) and/or 35-45 (w/v), with a nozzle temperature of 110-150° C., 120-140° C. and/or 125-135° C. and/or an outlet temperature of 60-80° C. and/or 65-70° C., and/or
   is spray-dried at a saccharide solution concentration of 20-60 (w/v), 30-50 (w/v) and/or 35-45 (w/v), with a nozzle temperature of 110-150° C., 120-140° C. and/or 125-135° C., and/or an outlet temperature of 60-80° C. and/or 65-70° C., and wherein the pH of said purified saccharide solution is ranging from 2 to 7, from 3 to 6 and/or from 4 to 5.
18. The purified saccharide solution, the purified saccharide or the purified saccharide mixture comprising a saccharide obtainable, preferably obtained, by a method according to any one of previous preferred embodiments.
19. Purified saccharide obtainable or obtained by a method according to any one of preferred embodiments 1 to 17, wherein the purified saccharide solution comprising said purified saccharide is i) dried, preferably spray-dried or dried via an agitated thin film dryer; ii) lyophilized; iii) crystallized or iv) concentrated to a syrup of at least 20% dry matter, at least 30% dry matter and/or at least 40% dry matter.
20. Saccharide purified according to the method according to any one of preferred embodiments 1 to 17 and wherein the purified saccharide obtained after said method:
   has an ash content of 10%, ≤9%, ≤8%, ≤7%, ≤6% and/or 5% on total dry solid,
   has an ash content of 10% on total dry solid with a lead content lower than 0.1 mg/kg dry solid,
   has an ash content of 10% on total dry solid with an arsenic content lower than 0.2 mg/kg dry solid,
   has an ash content of 10% on total dry solid with a cadmium content lower than 0.1 mg/kg dry solid,
   has an ash content of 10% on total dry solid with a mercury content lower than 0.5 mg/kg dry solid,
   has a protein content below 100 mg per kg dry solid, a DNA content below 10 ng per gram dry solid and/or an endotoxin content below 10000 EU per gram dry solid,
   is free of DNA, recombinant DNA, proteins, proteins derived from the recombinant micro-organism, and/or recombinant genetic material,
   has less than 125 μg/kg of 3-MCPD, wherein said 3-MCPD is the sum of 3-monochloropropanediol (3-MCPD) and 3-MCPD fatty acid esters,
   has less than 0.5 μg/kg epichlorohydrin, and/or
   has a conductivity of less than 10 mS/cm at a 300 g/L solution.
21. Dried powder of purified saccharide solution obtained from a method according to preferred embodiment 17, wherein said dried powder:
   contains ≤15%-wt., ≤10%-wt., ≤7%-wt. and/or ≤5%-wt. of water,
   has a mean particle size of 50 to 250 μm, 95 to 120 am and/or 110 to 120 μm, wherein said particle size is determined by laser diffraction,
   has an ash content of 10%, ≤9%, ≤8%, ≤7%, ≤6% and/or 5% on total dry solid,
   has an ash content of 10% on total dry solid with a lead content lower than 0.1 mg/kg dry solid,
   has an ash content of 10% on total dry solid with an arsenic content lower than 0.2 mg/kg dry solid,
   has an ash content of 10% on total dry solid with a cadmium content lower than 0.1 mg/kg dry solid,
   has an ash content of 10% on total dry solid with a mercury content lower than 0.5 mg/kg dry solid,
   has a protein content below 100 mg per kg dry solid, a DNA content below 10 ng per gram dry solid and/or an endotoxin content below 10000 EU per gram dry solid,
   is free of DNA, recombinant DNA, proteins, proteins derived from the recombinant micro-organism and/or recombinant genetic material,
   has less than 125 μg/kg of 3-MCPD, wherein said 3-MCPD is the sum of 3-monochloropropanediol (3-MCPD) and 3-MCPD fatty acid esters,
   has less than 0.5 μg/kg epichlorohydrin, and/or
   is a spray-dried powder.

The invention will be described in more detail in the examples. The following examples will serve as further illustration and clarification of the present invention and are not intended to be limiting.

### EXAMPLES

### Example 1. Materials and Methods

A. Escherichia coli

### Media and Cultivation

The Luria Broth (LB) medium consisted of 1% tryptone peptone (Difco, Erembodegem, Belgium), 0.5% yeast extract (Difco) and 0.5% sodium chloride (VWR. Leuven, Belgium). The minimal medium used in cultivation experiments in 96-well plates or in shake flasks contained 2.00 g/L NH₄Cl, 5.00 g/L (NH₄)₂SO₄, 2.993 g/L KH₂PO₄, 7.315 g/L κ₂HPO₄, 8.372 g/L MOPS, 0.5 g/L NaCl, 0.5 g/L MgSO₄·7H₂O, 30 g/L sucrose or 30 g/L glycerol, 1 ml/L vitamin solution, 100 μl/L molybdate solution, and 1 mL/L selenium solution. Depending on the experiment, 0.30 g/L sialic acid, 0.30 g/L GlcNAc, 20 g/L lactose, 20 g/L LacNAc (Gal 1-4GlcNAc), 20 g/L LNB (Galβ1-3GlcNAc), 20 g/L LN3 (GlcNAc(31-3Gal(31-4Glc), 20 g/L LNT (Gal(31-3GlcNAcβ1-3Galβ1-4Glc) and/or 20 g/L LNnT (Gal 31-4GlcNAc(31-3Galβ1-4Glc) were additionally added to the medium. The minimal medium was set to a pH of 7 with 1M KOH. Vitamin solution consisted of 3.6 g/L FeCl₂·4H₂O, 5.0 g/L CaCl₂·2H₂O, 1.3 g/L MnCl₂·2H₂O, 0.38 g/L CuCl₂·2H₂O, 0.5 g/L CoCl₂·6H₂O, 0.94 g/L ZnCl₂, 0.0311 g/L H₃BO₄, 0.4 g/L Na₂EDTA·2H₂O and 1.01 g/L thiamine·HCl. The molybdate solution contained 0.967 g/L NaMoO₄·2H₂O. The selenium solution contained 42 g/L Seo2. The minimal medium for fermentations contained 6.75 g/L NH₄Cl, 1.25 g/L (NH₄)₂SO₄, 2.93 g/L KH₂PO₄ and 7.31 g/L KH₂PO₄, 0.5 g/L NaCl, 0.5 g/L MgSO₄·7H₂O, 30 g/L sucrose or 30 g/L glycerol, 1 mL/L vitamin solution, 100 μL/L molybdate solution, and 1 mL/L selenium solution with the same composition as described above. As specified in the respective examples, 0.30 g/L sialic acid, 0.30 g/L GlcNAc, 20 g/L lactose, 20 g/L LacNAc, 20 g/L LNB, 20 g/L LN3, 20 g/L LNT and/or 20 g/L LNnT were additionally added to the medium. Complex medium was sterilized by autoclaving (121° C., 21 min) and minimal medium by filtration (0.22 μm Sartorius). When necessary, the medium was made selective by adding an antibiotic: e.g., chloramphenicol (20 mg/L), carbenicillin (100 mg/L), spectinomycin (40 mg/L) and/or kanamycin (50 mg/L).

A preculture of 96-well microtiter plate experiments was started from a cryovial, in 150 μL LB and was incubated overnight at 37° C. on an orbital shaker at 800 rpm. This culture was used as inoculum for a 96 well square microtiter plate, with 400 μL minimal medium by diluting 400×. These final 96-well culture plates were then incubated at 37° C. on an orbital shaker at 800 rpm for 72 h, or shorter, or longer. To measure sugar concentrations at the end of the cultivation experiment whole broth samples were taken from each well by boiling the culture broth for 15 min at 60° C. before spinning down the cells (=average of intra- and extracellular sugar concentrations).

A preculture for the bioreactor was started from an entire 1 mL cryovial of a certain strain, inoculated in 250 mL or 500 mL minimal medium in a 1 L or 2.5 L shake flask and incubated for 24 h at 37° C. on an orbital shaker at 200 rpm. A 5 L bioreactor was then inoculated (250 mL inoculum in 2 L batch medium); the process was controlled by MFCS control software (Sartorius Stedim Biotech, Melsungen, Germany). Culturing condition were set to 37° C., and maximal stirring; pressure gas flow rates were dependent on the strain and bioreactor. The pH was controlled at 6.8 using 0.5 M H₂SO₄ and 20% NH₄OH. The exhaust gas was cooled. 10% solution of silicone antifoaming agent was added when foaming raised during the fermentation.

### Strains and Mutations

Escherichia coli K12 MG1655 [λ-, F-, rph-1] was obtained from the Coli Genetic Stock Center (US), CGSC Strain #: 7740, in March 2007. Gene disruptions, gene introductions and gene replacements were performed using the technique published by Datsenko and Wanner (PNAS 97 (2000), 6640-6645). All constitutive promoters, UTRs and terminator sequences originated from the libraries described by Cambray et al. (Nucleic Acids Res. 2013, 41(9), 5139-5148), Dunn et al. (Nucleic Acids Res. 1980, 8, 2119-2132), Edens et al. (Nucleic Acids Res. 1975, 2, 1811-1820), Kim and Lee (FEBS Letters 1997, 407, 353-356) and Mutalik et al. (Nat. Methods 2013, No. 10, 354-360). Genes were ordered synthetically at Twist Bioscience (twistbioscience.com) or IDT (eu.idtdna.com) and the codon usage was adapted using the tools of the supplier. All strains were stored in cryovials at −80° C. (overnight LB culture mixed in a 1:1 ratio with 70% glycerol).

B. Saccharomyces cerevisiae

### Media and Cultivation

Strains were grown on Synthetic Defined yeast medium with Complete Supplement Mixture (SD CSM) or CSM drop-out (SD CSM-Ura, SD CSM-Trp, SD CSM-His) containing 6.7 g/L Yeast Nitrogen Base without amino acids (YNB w/o AA, Difco), 20 g/L agar (Difco) (solid cultures), 22 g/L glucose monohydrate or 20 g/L lactose and 0.79 g/L CSM or 0.77 g/L CSM-Ura, 0.77 g/L CSM-Trp, or 0.77 g/L CSM-His (MP Biomedicals).

In general, yeast strains were initially grown on SD CSM plates to obtain single colonies. These plates were grown for 2-3 days at 30° C. Starting from a single colony, a preculture was grown over night in 5 mL at 30° C., shaking at 200 rpm. Subsequent 125 mL shake flask experiments were inoculated with 2% of this preculture, in 25 mL media. These shake flasks were incubated at 30° C. with an orbital shaking of 200 rpm.

### Strains, Plasmids and Mutations

S. cerevisiae BY4742 created by Brachmann et al. (Yeast (1998) 14:115-32) was used, available in the Euroscarf culture collection. All mutant strains were created by homologous recombination or plasmid transformation using the method of Gietz (Yeast 11:355-360, 1995). Genes were expressed using synthetic constitutive promoters, as described by e.g., Blazeck (Biotechnology and Bioengineering, Vol. 109, No. 11, 2012), Redden and Alper (Nat. Commun. 2015, 6, 7810), Liu et al. (Microb. Cell Fact. 2020, 19, 38), Xu et al. (Microb. Cell Fact. 2021, 20, 148) and Lee et al. (ACS Synth. Biol. 2015, 4(9), 975-986).

C. Bacillus subtilis

### Media and Cultivation

Two media are used to cultivate B. subtilis: i.e., a complex medium like a rich Luria Broth (LB) and a minimal medium for shake flask cultures. The LB medium consisted of 1% tryptone peptone (Difco), 0.5% yeast extract (Difco) and 0.5% sodium chloride (VWR). Luria Broth agar (LBA) plates consisted of the LB media, with 12 g/L agar (Difco) added. The minimal medium contained 2.00 g/L (NH₄)₂SO₄, 7.5 g/L KH₂PO₄, 17.5 g/L κ₂HPO₄, 1.25 g/L Na-citrate, 0.25 g/L MgSO₄·7H₂O, 0.05 g/L tryptophan, from 10 up to 30 g/L glucose (or another carbon source including but not limited to fructose, maltose, sucrose, glycerol and maltotriose), 10 mL/L trace element mix and 10 mL/L Fe-citrate solution. The medium was set to a pH of 7 with 1 M KOH. Depending on the experiment lactose is added as a precursor. The trace element mix consisted of 0.735 g/L CaCl₂·2H₂O, 0.1 g/L MnCl₂·2H₂O, 0.033 g/L CuCl₂·2H₂O, 0.06 g/L CoCl₂·6H₂O, 0.17 g/L ZnCl₂, 0.0311 g/L H₃BO₄, 0.4 g/L Na₂EDTA·2H₂O and 0.06 g/L Na₂MoO₄. The Fe-citrate solution contained 0.135 g/L FeCl₃·6H₂O, 1 g/L Na-citrate (Hoch 1973 PMC1212887). Complex medium, e.g., LB, was sterilized by autoclaving (121° C., 21 min) and minimal medium by filtration (0.22 μm Sartorius). When necessary, the medium was made selective by adding an antibiotic.

B. subtilis strains were initially grown on LB agar to obtain single colonies. These plates were grown over night at 37° C. Starting from a single colony, a preculture was grown over night in 5 mL at 37° C., shaking at 200 rpm. Subsequent 125 mL shake flask experiments were inoculated with 2% of this preculture, in 25 mL media. These shake flasks were incubated at 37° C. with an orbital shaking of 200 rpm for 72 h, or shorter of longer. At the end of the cultivation experiment samples were taken to measure the supernatant concentration (extracellular sugar concentrations, after 5 min. spinning down the cells), or by boiling the culture broth for 15 min at 90° C. or for 60 min at 60° C. before spinning down the cells (=whole broth concentration, i.e., intra- and extracellular sugar concentrations).

### Strains, Plasmids and Mutations

B. subtilis 168 is used as available at the Bacillus Genetic Stock Center (Ohio, USA). Plasmids for gene deletion via Cre/lox are constructed as described by Yan et al. (Appl & Environm microbial, September 2008, p 5556-5562). Gene disruption is done via homologous recombination with linear DNA and transformation via the electroporation as described by Xue et al. (J. microb. Meth. 34 (1999) 183-191). The method of gene knockouts is described by Liu et al. (Metab. Engine. 24 (2014) 61-69). Integrative vectors as described by Popp et al. (Sci. Rep., 2017, 7, 15158) are used as expression vector and could be further used for genomic integrations if necessary. A suitable promoter for expression can be derived from the part repository (iGem): sequence id: BBa_K143012, BBa_K823000, BBa_K823002 or BBa_K823003. Cloning can be performed using Gibson Assembly, Golden Gate assembly, Cliva assembly, LCR or restriction ligation.

D. Corynebacterium glutamicum

### Media and Cultivation

Two different media are used, namely complex medium like e.g., a rich tryptone-yeast extract (TY) medium, and a minimal medium for shake flask (MMsf). The minimal medium uses a 1000× stock trace element mix. Trace element mix consisted of 10 g/L CaCl₂, 10 g/L FeSO₄·7H₂O, 10 g/L MnSO₄·H₂O, 1 g/L ZnSO₄·7H₂O, 0.2 g/L CuSO₄, 0.02 g/L NiCl₂·6H₂O, 0.2 g/L biotin (pH 7) and 0.03 g/L protocatechuic acid.

The minimal medium for the shake flasks (MMsf) experiments contained 20 g/L (NH₄)₂SO₄, 5 g/L urea, 1 g/L KH₂PO₄, 1 g/L κ₂HPO₄, 0.25 g/L MgSO₄·7H₂O, 42 g/L MOPS, from 10 up to 30 g/L glucose or another carbon source including but not limited to fructose, maltose, sucrose, glycerol and maltotriose when specified in the examples and 1 ml/L trace element mix. Depending on the experiment lactose, LNB, and/or LacNAc could be added to the medium. The TY medium consisted of 1.6% tryptone (Difco, Erembodegem, Belgium), 1% yeast extract (Difco) and 0.5% sodium chloride (VWR. Leuven, Belgium). TY agar (TYA) plates consisted of the TY media, with 12 g/L agar (Difco, Erembodegem, Belgium) added. Complex medium, e.g., TY, was sterilized by autoclaving (121° C., 21 min) and minimal medium by filtration (0.22 μm Sartorius). When necessary, the medium was made selective by adding an antibiotic.

A preculture was started from a cryovial or a single colony from a TY plate, in 6 mL TY and was incubated overnight at 37° C. on an orbital shaker at 200 rpm. Subsequent 125 mL shake flask experiments were inoculated with 2% of this preculture, in 25 mL MMsf medium. These shake flasks were incubated at 37° C. with an orbital shaking of 200 rpm for 72 h, or shorter of longer. At the end of the cultivation experiment samples were taken to measure the supernatant concentration (extracellular sugar concentrations, after 5 min. spinning down the cells), or by boiling the culture broth for 15 min at 90° C. or for 60 min at 60° C. before spinning down the cells (=whole broth concentration, i.e., intra- and extracellular sugar concentrations).

### Strains and Mutations

Corynebacterium glutamicum was used as available at the American Type Culture Collection (ATCC 13032). Integrative plasmid vectors were made using the Cre/loxP technique as described by Suzuki et al. (Appl. Microbiol. Biotechnol., 2005 April, 67(2):225-33) and temperature-sensitive shuttle vectors as described by Okibe et al. (Journal of Microbiological Methods 85, 2011, 155-163) are constructed for gene deletions, mutations and insertions. Suitable promoters for (heterologous) gene expression can be derived from Yim et al. (Biotechnol. Bioeng., 2013 November, 110(11):2959-69). Cloning can be performed using Gibson Assembly, Golden Gate assembly, Cliva assembly, LCR or restriction ligation.

### E. Optical Density

Cell density of the cultures was frequently monitored by measuring optical density at 600 nm (Implen Nanophotometer NP80, Westburg, Belgium or with a Spark 10M microplate reader, Tecan, Switzerland). The maximum growth speed (mumax) was calculated based on the observed optical densities at 600 nm using the R package grofit.

### F. Heterologous and Homologous Expression

Genes that needed to be expressed, be it from a plasmid or from the genome were synthetically synthetized with one of the following companies: IDT or Twist Bioscience. Proteins described in present disclosure are summarized in Table 1. Unless stated otherwise, the UniProt IDs of the proteins described correspond to their sequence version 01 as present in the UniProt Database version release 202103 of 9 Jun. 2021. Expression could be further facilitated by optimizing the codon usage to the codon usage of the expression host. Genes were optimized using the tools of the supplier.

TABLE 1
Overview of proteins with corresponding UniProt IDs (sequence version 01,
UniProt Database 2021_03 of 9 Jun. 2021) as described in present disclosure

| | | | | Country of origin of |
|---|---|---|---|---|
| | | | | digital sequence |
| UniProt ID | Name | Organism | Origin | information |
| A7LVG6 | AGE | Bacteroides ovatus | Synthetic | USA |
| A0ZZH6 | BaSP | Bifidobacterium adolescentis | Synthetic | Germany |
| E0IXR1 | cscB | Escherichia coli W | Synthetic | USA |
| Q03417 | Frk | Zymomonas mobilis | Synthetic | United Kingdom |
| P09147 | galE | Escherichia coli K-12 | Synthetic | USA |
| | | MG1655 | | |
| P17169* | glmS | Escherichia coli K-12 | Synthetic | USA |
| | | MG1655 | | |
| O30511 | HpFucT | Helicobacter pylori UA1234 | Synthetic | Canada |
| P07921 | LAC12 | Kluyveromyces lactis | Synthetic | USA |
| P02920 | LacY | Escherichia coli K-12 | Synthetic | USA |
| | | MG1655 | | |
| Q9JXQ6 | LgtA | Neisseria meningitidis MC58 | Synthetic | United Kingdom |
| Q51116** | LgtB | Neisseria meningitidis MC58 | Synthetic | United Kingdom |
| A0A849CI62 | NeuA | Pasteurella multocida | Synthetic | USA |
| E0NCD4 | NeuB | Neisseria meningitidis | Synthetic | United Kingdom |
| O66375 | PdST6 | Photobacterium damselae | Synthetic | Japan |
| Q9CLP3 | PmultST3 | Pasteurella multocida | Synthetic | USA |
| P0A840 | SurE | Escherichia coli K-12 | Synthetic | USA |
| | | MG1655 | | |
| D3QY14 | wbgO | Escherichia coli O55:H7 | Synthetic | Germany |

*Sequence version 04 (23 Jan. 2007) as present in the UniProt Database 2021_03 of 9 Jun. 2021
**Sequence version 02 (1 Dec. 2000) as present in the UniProt Database 2021_03 of 9 Jun. 2021

### G. Analytical Analysis

Standards such as but not limited to sucrose, lactose, 3′SL, 6′SL, lacto-N-triose II (LN3), lacto-N-tetraose (LNT), lacto-N-neo-tetraose (LNnT), LNFP-I, LNFP-II, LNFP-III, LNFP-V, LNFP-VI, LSTa, LSTc and LSTd were purchased from Carbosynth (UK), Elicityl (France) and IsoSep (Sweden). Other compounds were analyzed with in-house made standards.

Neutral oligosaccharides were analyzed on a Waters Acquity H-class UPLC with Evaporative Light Scattering Detector (ELSD) or a Refractive Index (RI) detection. A volume of 0.7 μL sample was injected on a Waters Acquity UPLC BEH Amide column (2.1×100 mm; 130 Å; 1.7 μm) column with an Acquity UPLC BEH Amide VanGuard column, 130 Å, 2.1×5 mm. The column temperature was 50° C. The mobile phase consisted of a ¼ water and ¾ acetonitrile solution to which 0.2% triethylamine was added. The method was isocratic with a flow of 0.130 mL/min. The ELSD detector had a drift tube temperature of 50° C. and the N2 gas pressure was 50 psi, the gain 200 and the data rate 10 pps. The temperature of the RI detector was set at 35° C.

Sialylated oligosaccharides were analyzed on a Waters Acquity H-class UPLC with Refractive Index (RI) detection. A volume of 0.5 μL sample was injected on a Waters Acquity UPLC BEH Amide column (2.1 ×100 mm; 130 Å; 1.7 μm). The column temperature was 50° C. The mobile phase consisted of a mixture of 70% acetonitrile, 26% ammonium acetate buffer (150 mM) and 4% methanol to which 0.05% pyrrolidine was added. The method was isocratic with a flow of 0.150 mL/min. The temperature of the RI detector was set at 35° C.

Both neutral and sialylated sugars were analyzed on a Waters Acquity H-class UPLC with Refractive Index (RI) detection. A volume of 0.5 μL sample was injected on a Waters Acquity UPLC BEH Amide column (2.1×100 mm; 130 Å; 1.7 μm). The column temperature was 50° C. The mobile phase consisted of a mixture of 72% acetonitrile and 28% ammonium acetate buffer (100 mM) to which 0.1% triethylamine was added. The method was isocratic with a flow of 0.260 mL/min. The temperature of the RI detector was set at 35° C.

For analysis on a mass spectrometer, a Waters Xevo TQ-MS with Electron Spray Ionisation (ESI) was used with a desolvation temperature of 450° C., a nitrogen desolvation gas flow of 650 L/h and a cone voltage of 20 V. The MS was operated in selected ion monitoring (SIM) in negative mode for all oligosaccharides. Separation was performed on a Waters Acquity UPLC with a Thermo Hypercarb column (2.1×100 mm; 3 μm) on 35° C. A gradient was used wherein eluent A was ultrapure water with 0.1% formic acid and wherein eluent B was acetonitrile with 0.1% formic acid. The oligosaccharides were separated in 55 min using the following gradient: an initial increase from 2 to 12% of eluent B over 21 min, a second increase from 12 to 40% of eluent B over 11 min and a third increase from 40 to 100% of eluent B over 5 min. As a washing step 100% of eluent B was used for 5 min. For column equilibration, the initial condition of 2% of eluent B was restored in 1 min and maintained for 12 min.

Both neutral and sialylated sugars at low concentrations (below 50 mg/L) were analyzed on a Dionex HPAEC system with pulsed amperometric detection (PAD). A volume of 5 μL of sample was injected on a Dionex CarboPac PA200 column 4×250 mm with a Dionex CarboPac PA200 guard column 4×50 mm. The column temperature was set to 30° C. A gradient was used wherein eluent A was deionized water, wherein eluent B was 200 mM Sodium hydroxide and wherein eluent C was 500 mM Sodium acetate. The oligosaccharides were separated in 60 min while maintaining a constant ratio of 25% of eluent B using the following gradient: an initial isocratic step maintained for 10 min of 75% of eluent A, an initial increase from 0 to 4% of eluent C over 8 min, a second isocratic step maintained for 6 min of 71% of eluent A and 4% of eluent C, a second increase from 4 to 12% of eluent C over 2.6 min, a third isocratic step maintained for 3.4 min of 63% of eluent A and 12% of eluent C and a third increase from 12 to 48% of eluent C over 5 min. As a washing step 48% of eluent C was used for 3 min. For column equilibration, the initial condition of 75% of eluent A and 0% of eluent C was restored in 1 min and maintained for 11 min. The applied flow was 0.5 mL/min.

### H. Ash Content Measurement

The ash content within a sample can be measured by methods like e.g., dry ashing, wet ashing or low temperature plasma dry ashing. The sample is weighed before and after ashing to determine the concentration of ash present. The ash content can be expressed on dry basis and is calculated by dividing the mass of the ashed material by the mass of the dry material before ashing. Multiplied with 100, this gives the percentage of ash in the material. In a similar way the wet ash percentage can be determined for liquid products, wherein the mass of the liquid before and after ashing is used instead of the mass of the dry material.

In examples further described, the ash content was determined gravimetrically. For each sample, a porcelain crucible was pre-heated at 500° C. for 30 minutes. Afterwards, it was cooled to room temperature in a desiccator containing anhydrous silica. When cooled, the crucible was weighed with 0.1 mg accuracy.

Next, 5 g of oligosaccharide (e.g., HMO) powder was weighed in the crucible and charred using a Bunsen burner. Afterwards, the charred crucibles were put in a muffle furnace at 500° C. for 4 hours. They were then again cooled to room temperature and weighed. The ash content was determined according the equation=(mass of crucible−mass of charred crucible)/(mass of oligosaccharide powder)*100%.

### Heavy Metal Determination

A robust general inductively coupled plasma-mass spectrometry (ICP-MS) based method was used for the detection and quantitation for each of the following elements: arsenic (As), selenium (Se), cadmium (Cd), tin (Sn), lead (Pb), silver (Ag), palladium (Pd), platinum (Pt), mercury (Hg), molybdenum (Mo), sodium (Na), potassium (K), Calcium (Ca), Magnesium (Mg), Iron (Fe), zinc (Zn), manganese (Mn), Phosphorus (P), selenium (Se).

Nitric acid (>65%, Sigma-Aldrich) was used for microwave digestion and standard/sample preparation. All dilutions were done using 18.2 MO-cm (Millipore, Bedford, MA, USA) de-ionized water (DIW). About 0.2 g of each sample were digested in 5 mL of HNO3 using the microwave digestion (CEM, Mars 6) program 15 minutes (min) ramping time and 15 min holding time at 100 W and 50° C. followed by 15 min ramping time and 20 min holding time at 1800 W and 210° C. The samples were cooled after digestion for 30 minutes. The fully digested samples were then diluted to 50 mL with DIW.

Analyses were carried out using a standard Agilent 7800 ICP-MS, which includes the fourth-generation ORS cell system for effective control of polyatomic interferences using helium collision mode (He mode). The ORS controls polyatomic interferences using He to reduce the transmission of all common matrix-based polyatomic interferences. Smaller, faster analyte ions are separated from larger, slower interference-ions using kinetic energy discrimination (KED). All elements, except Se, were measured in He mode with a flow rate of 5 mL/min. Se was measured in High Energy He (HEHe) mode, using a cell gas flow rate of 10 mL/min. The 7800 ICP-MS was configured with the standard sample introduction system consisting of a MicroMist glass concentric nebulizer, quartz spray chamber, quartz torch with 2.5 mm i.d. injector, and nickel interface cones. The ICP-MS operating conditions are: 1550 W RF power, 8 mm sampling depth, 1.16 I/min nebulizing gas, autotuned lens tuning, 5 or 10 ml/min helium gas flow, 5 V KED.

### Dry Matter and Moisture Content Quantification

Sartorius MA150 Infrared Moisture Analyzer is used to determine the dry matter content of the oligosaccharide(s). 0.5 g of oligosaccharide is weighed on an analytical balance and is dried in the infrared moisture analyzer until the weight of the sample is stable. The mass of the dried sample divided by the mass of the sample before drying gives the dry matter content (in percent) of the oligosaccharide(s) or sample including oligosaccharide(s). In a similar way a liquid sample is weighed, however, the amount of liquid weighed is adapted to the expected amount of dry matter in the liquid, so the mass of the dry matter is properly measurable on an analytical balance.

A moisture analyzer measures the dry matter, but not the water content. Karl Fisher titration is used to determine the amount of water present in a powder, ingredient of food. The KF titration is carried out with a Karl Fischer titrator DL31 from Mettler Toledo using the two-component technique with Hydra-Point Solvent G and Hydra-Point titrant (5 mg H2O/mL), both purchased from J. T. Baker (Deventer, Holland). The polarising current for bipotentiometric end-point determination was 20 μA and the stop voltage 100 mV. The end-point criterion was the drift stabilisation (15 μg H2O/min) or maximum titration time (10 min).

The moisture content (MC) of sample was calculated using the following equation: MC=V_KF W_eq 100/W_sample; where V_KF is the consumption of titrant in mL, W_eq the titer of titrant in mg H2O/mL and W_sample the weight of sample in mg.

### I. Biomass Dry Mass Content (Cell Dry Mass)

Cell dry weight (CDW) was obtained by centrifugation (15 min, 5000 g) of 20 g broth in pre-dried (70° C. overnight) and weighted falcons. The pellets were subsequently washed once with 20 mL physiological solution (9 g/L NaCl) and dried at 70° C. to a constant weight. The final weight was corrected for the added sodium chloride to the sample.

An alternative method for determination of cell dry mass or cell dry weight, also well known to a person skilled in the art, uses an Infrared Moisture Analyzer (Sartorius MA150). The equipment was allowed to warm-up for 30 min, and the standby temperature was set to 60° C. The balance was automatically tared with the weighing aluminium pan containing a glass microfibre filter pad (0.2 μm) after drying (105° C. in moisture analyser to achieve stable weight, lasting about 1 min) and equilibration. The drying temperature was set to 105° C. Samples were evenly added to the glass microfibre pad, and the drying programme was set to end when the weight change was less than 0.1 mg min⁻¹. The dried glass fibre filter pad was used to filter off the biomass from 10 mL of broth and the filtered broth was washed 2 times with physiological solution. The filter was then dried again by means of the method described above, drying the already tared aluminium pan and the filter pad at 105° C. until stable weight.

### J. Protein Quantification

For protein quantification a method is used that is compatible with reducing agents, such as reducing sugars or oligosaccharides with a reducing end. To this end, a Bradford assay (Thermo Scientific, Pierce) was used with a linear range between 1 and 1500 μg/mL. The assay was calibrated with a standard curve of BSA. The protein content of dried oligosaccharide products was quantified by dissolving a pre-weighed quantity in 18.2 MO-cm (Millipore, Bedford, MA, USA) de-ionized water (DIW) up to a quantity of 50% (m/v). The amount of protein is measured at 595 nm and converted to concentration with the calibration curve based on BSA.

### K. DNA Quantification

Production host specific DNA residue is quantified by RT-qPCR, for which specific primers on the host are designed so that residual DNA of the production host is amplified. The RT-qPCR was performed according to the standard protocol of a kit obtained from Sigma and was based on SYBR Green detection.

Total DNA is measured by means of a Threshold assay (Molecular Devices), based on an immunoassay allowing to measure as low as 2 μg of DNA in a sample in solution. Double stranded DNA is measured by means of the SpectraMax® Quant™ AccuBlue™ Pico dsDNA Assay Kit (Molecular Devices) having a linear range between 5 μg and 3 ng of dsDNA.

### L. Endotoxin Measurement

Endotoxin in the liquid was measured by means of a limulus amebocyte lysate (LAL) test like e.g., from Lonza; Genscript or ThermoFisher according to the protocol as set out by the manufacturer.

### M. Laser Diffraction

The powder particle size can be assessed by laser diffraction. The system detects scattered and diffracted light by an array of concentrically arranged sensor elements. The software-algorithm is then approximating the particle counts by calculating the z-values of the light intensity values, which arrive at the different sensor elements. The analysis can be executed using a SALD-7500 Aggregate Sizer (Shimadzu Corporation, Kyoto, Japan) quantitative laser diffraction system (qLD).

A small amount (spatula tip) of each sample can be dispersed in 2 mL isooctane and homogenized by ultrasonication for five minutes. The dispersion will then be transferred into a batch cell filled with isooctane and analyzed in manual mode.

Data acquisition settings can be as follows: Signal Averaging Count per Measurement: 128, Signal Accumulation Count: 3, and Interval: 2 seconds.

Prior to measurement, the system can be blanked with isooctane. Each sample dispersion will be measured 3 times, and the mean values and the standard deviation will be reported. Data can be evaluated using software WING SALD II version V3.1. When the refractive index of the sample is unknown, the refractive index of sugar (disaccharide) particles (1.530) can be used for determination of size distribution profiles. Size values for mean and median diameter are reported. The mean particle sizes for all samples are very similar due to the spray dryer settings used. In addition, the particle size distribution will show the presence of one main size population for all the samples.

### N. Color Determination

Color was determined by filtering 1 mL of a saccharide solution of 10 Brix over a 0.45 μm syringe filter and afterwards measuring the absorbance of this solution at a wavelength of 430 nm.

### Example 2. Synthesis of a Saccharide or a Mixture Comprising a Saccharide with a Modified Cell

An E. coli strain engineered for production of 6′SL or 3′SL as described in WO2018122225 was used in a fed-batch fermentation process. Fed-batch fermentations at bioreactor scale were performed as described in Example 1. Sucrose was used as a carbon source and lactose was added in the batch medium. During fed-batch, sucrose was added via an additional feed. Regular broth samples were taken at several time points during the fermentation process and the 6′SL or 3′SL produced, respectively, was measured using UPLC as described in Example 1. Typically, the obtained product concentration was above 50 g/L.

An E. coli K12 MG1655 strain modified for production of lacto-N-triose (LN3, GlcNAc-β1,3-Gal-β1,4-Glc) as described e.g., in WO22034075 was further modified to express 1) the N-acetylglucosamine beta-1,3-galactosyltransferase WbgO (Uniprot ID D3QY14) from E. coli O55:H7 to produce lacto-N-tetraose (LNT, Gal-β1,3-GlcNAc-β1,3-Gal-β1,4-Glc) or 2) the N-acetylglucosamine beta-1,4-galactosyltransferase LgtB (Uniprot ID Q51116, sequence version 02, 1 Dec. 2000) from Neisseria meningitidis to produce lacto-N-neotetraose (LNnT, Gal-β1,4-GlcNAc-β1,3-Gal-β1,4-Glc), respectively. Fed-batch fermentations at bioreactor scale were performed as described in Example 1. Sucrose was used as a carbon source and lactose was added in the batch medium. During fed-batch, sucrose was added via an additional feed. Regular broth samples were taken at several time points during the fermentation process and the LNT or LNnT produced, respectively, was measured using UPLC as described in Example 1. Typically, the obtained product concentration was above 50 g/L.

An E. coli K12 MG1655 strain engineered for production of 2′FL (Fuc-α1,2-Gal-β1,4-Glc) as described e.g., in WO22129470 was evaluated in a batch and in a fed-batch fermentation process. Fed-batch fermentations at bioreactor scale (5 and 30 L) were performed as described in Example 1. In these examples, sucrose was used as a carbon source and lactose was added in the batch medium as a precursor. Regular broth samples were taken and the production of 2′FL was measured using UPLC as described in Example 1. Typically, the obtained product concentration was above 50 g/L.

An E. coli K12 MG1655 strain engineered for production of 3-FL (Gal-β1,4-[Fuc-α1,3]-Glc) as described e.g., in WO20127417 was evaluated in a batch and in a fed-batch fermentation process. Fed-batch fermentations at bioreactor scale (5 and 30 L) were performed as described in Example 1. In these examples, sucrose was used as a carbon source and lactose was added in the batch medium as a precursor. Regular broth samples were taken and the production of 2′FL was measured using UPLC as described in Example 1. Typically, the obtained product concentration was above 50 g/L.

An E. coli K12 MG1655 strain engineered for production of an oligosaccharide mixture comprising 2′FL (Fuc-α1,2-Gal-β1,4-Glc), 3-FL (Gal-β1,4-[Fuc-α1,3]-Glc) and DiFL (Fuc-α1,2-Gal-β1,4-[Fuc-α1,3]-Glc) as described e.g., in WO22034067 was evaluated in a batch and in a fed-batch fermentation process. Fed-batch fermentations at bioreactor scale (5 and 30 L) were performed as described in Example 1. In these examples, sucrose was used as a carbon source and lactose was added in the batch medium as a precursor. Regular broth samples were taken and the production of 2′FL, 3-FL and DiFL was measured using UPLC as described in Example 1. The experiment demonstrated that broth samples taken at the end of batch phase comprised an oligosaccharide mixture of 2′FL and 3-FL together with unmodified lactose, whereas broth samples taken at the end of the fed-batch phase comprised an oligosaccharide mixture of 2′FL, 3-FL and DiFL. As the ratios of lactose, 2′FL, 3-FL and DiFL changed over time during fed-batch, they could be manipulated during the fermentation process by discontinuation of the fermentation process at a desired time in fed-batch phase. Typically, the obtained product concentration was above 50 g/L.

E. coli K12 MG1655 strains modified for production of i) an oligosaccharide mixture comprising 2′FL, DiFL, LN3, LNT, LNFP-I and LNFP-II or ii) an oligosaccharide mixture comprising 2′FL, 3-FL, DiFL, 3′SL, 6′SL, 3'S-2′FL, 3'S-3-FL, 6'S-2′FL, 6'S-3-FL, LN3, 3′SLN3, 6'S-LN3, LNT, LNFP-I and LSTa as described e.g., in WO22034067 were evaluated in a batch and in a fed-batch fermentation process. Fed-batch fermentations at bioreactor scale (5 and 30 L) were performed as described in Example 1. In these examples, sucrose was used as a carbon source and lactose was added in the batch medium as a precursor. Regular broth samples of each fermentation were taken and evaluated via UPLC for production of said oligosaccharide mixtures. Typically, the obtained product concentration was above 50 g/L.

A S. cerevisiae strain is engineered for production of 3′SL as described in Example 1 with a compatible yeast expression plasmid comprising constitutive transcriptional units for the lactose permease LAC12 from K. lactis (UniProt ID P07921), the glmS from E. coli (UniProt ID P17169, sequence version 04 (23 Jan. 2007)), the phosphatase SurE from E. coli (UniProt ID POA840), the N-acylglucosamine 2-epimerase AGE from B. ovatus (UniProt ID A7LVG6), the N-acetylneuraminate synthase NeuB from N. meningitidis (UniProt ID EONCD4), the N-acylneuraminate cytidylyltransferase NeuA from P. multocida (UniProt ID A0A849C162) and the alpha-2,3-sialyltransferase PmultST3 from P. multocida (UniProt ID Q9CLP3). Cultivation of the novel strain is performed according to the culture conditions provided in Example 1 using appropriate selective medium comprising lactose. Regular samples are taken and evaluated via UPLC for production of 3′SL.

In another example a S. cerevisiae strain is engineered as described in Example 1 with a first compatible yeast expression plasmid comprising constitutive transcriptional units for the lactose permease LAC12 from K. lactis (UniProt ID P07921), the glmS from E. coli (UniProt ID P17169, sequence version 04 (23 Jan. 2007)), the phosphatase SurE from E. coli (UniProt ID POA840), the N-acylglucosamine 2-epimerase AGE from B. ovatus (UniProt ID A7LVG6), the N-acetylneuraminate synthase NeuB from N. meningitidis (UniProt ID EONCD4), the N-acylneuraminate cytidylyltransferase NeuA from P. multocida (UniProt ID A0A849C162) and the alpha-2,6-sialyltransferase (PdST6) from P. damselae (UniProt ID 066375), and with a second compatible yeast expression plasmid comprising constitutive transcriptional units for the UDP-glucose-4-epimerase galE from E. coli (UniProt ID P09147), the galactoside beta-1,3-N-acetylglucosaminyltransferase LgtA from N. meningitidis (UniProt ID Q9JXQ6) and the N-acetylglucosamine beta-1,4-galactosyltransferase LgtB from N. meningitidis (Uniprot ID Q51116, sequence version 02, 1 Dec. 2000). Cultivation of the novel strain is performed according to the culture conditions provided in Example 1 using appropriate selective medium comprising lactose. Regular samples are taken and evaluated via UPLC for production of an oligosaccharide mixture comprising 6′SL, LN3, LNnT and LSTc.

A wild-type B. subtilis strain modified for production of sialic acid and CMP-sialic acid as described e.g., in WO22034067 is further modified with transcriptional units encoding the lactose permease LacY (UniProt ID P02920) from E. coli and the alpha-2,6-sialyltransferase (PdST6) from P. damselae (UniProt ID 066375). Cultivation of the novel strain is performed according to the culture conditions provided in Example 1 using appropriate selective medium comprising lactose. Regular samples are taken and evaluated via UPLC for production 6′SL.

A C. glutamicum strain modified for production of LN3 as described e.g., in WO22034069 is further modified for LNnT production with a transcriptional unit encoding the N-acetylglucosamine beta-1,4-galactosyltransferase LgtB from N. meningitidis (Uniprot ID Q51116, sequence version 02, 1 Dec. 2000). In a next step, the mutant strain is further modified with transcriptional units encoding the sucrose transporter (CscB) from E. coli W (UniProt ID E0IXR1), the fructose kinase (Frk) from Z. mobilis (UniProt ID Q03417) and the sucrose phosphorylase (BaSP) from B. adolescentis (UniProt ID A0ZZH6) enabling the strain to grow on sucrose. In a final step, the mutant strain is modified with a transcriptional unit encoding the alpha-1,3-fucosyltransferase HpFucT from H. pylori(UniProt ID 030511). Cultivation of the novel strain is performed according to the culture conditions provided in Example 1 using MMsf medium comprising lactose. Regular samples are taken and evaluated via UPLC for production of an oligosaccharide mixture comprising 3-FL, LN3, LNnT and LNFP-III.

### Example 3. Purification of 2′FL from a Fermentation Broth Obtained with an Engineered E. coli Strain

Broth comprising 2′FL was produced via fermentation as described in Example 2. After fermentation, fermentation broth comprising 2′FL and antifoam was subjected to heat treatment above 60° C. In a next step, the fermentation broth was diluted in a 1:1 ratio with demineralized water. Afterwards, the fermentation broth was subjected to filtration with a polypiperazine-amide membrane (TriSep UA60, Mann+Hummel) at pH 6.8 and at a temperature of 45° C. using an inlet pressure ranging from 5-9 bar. During the first part of the filtration the retentate was concentrated back to its original volume. Furthermore, the retentate was subjected to diafiltration on said polypiperazine-amide membrane at this constant volume until a total of 3.9 diafiltrations was obtained. The average flux during the filtration was 11.83 L/h/m². Said filtration retained the biomass and the antifoam in the retentate and resulted into the formation of a permeate comprising said 2′FL. The permeate was collected with a 2′FL recovery of 90%. The resulting permeate was then subjected to process steps well known in the art, such as nanofiltration, treatment with activated charcoal and drying to form a white powder of 2′FL, with a protein content below 100 mg per kg dry solid, a DNA content below 10 ng per gram dry solid and an endotoxin content below 10000 EU per gram dry solid.

### Example 4. Purification of 2′FL from a Fermentation Broth Obtained with an Engineered E. coli Strain

Broth comprising 2′FL was produced via fermentation as described in Example 2. After fermentation, fermentation broth comprising 2′FL and antifoam was subjected to heat treatment above 60° C. Afterwards, the fermentation broth was subjected to filtration with a polypiperazine-amide membrane (TriSep UA60, Mann+Hummel) at pH 6.5 and at a temperature of 45° C. using an inlet pressure of 8 bar.

The fermentation broth was directly diafiltrated at a constant volume until a total of 3.9 diafiltrations was obtained. The average flux during the filtration was 18.71 L/h/m². Said filtration retained the biomass and the antifoam in the retentate and resulted into the formation of a permeate comprising said 2′FL. The permeate was collected with a 2′FL recovery of 91.6%. The resulting permeate was then subjected to process steps well known in the art, such as nanofiltration, treatment with activated charcoal and drying to form a white powder of 2′FL, with a protein content below 100 mg per kg dry solid, a DNA content below 10 ng per gram dry solid and an endotoxin content below 10000 EU per gram dry solid.

### Example 5. Purification of 3′SL from a Fermentation Broth Obtained with an Engineered E. coli Strain

Broth comprising 3′SL was produced via fermentation as described in Example 2. After fermentation, fermentation broth comprising 3′SL and antifoam was subjected to heat treatment above 60° C. Afterwards, the fermentation broth was subjected to filtration with a polypiperazine-amide membrane (TriSep UA60, Mann+Hummel) at pH 6.8 and at a temperature of 40° C. using an inlet pressure of 9 bar. During the first part of the filtration the retentate was concentrated back to 50% of its original value. Furthermore, the retentate was subjected to diafiltration on said polypiperazine-amide membrane at a constant volume until a total of 2.6 diafiltrations was obtained. The average flux during the filtration was 10.30 L/h/m². Said filtration retained the biomass and the antifoam in the retentate and resulted into the formation of a permeate comprising said 3′SL. The permeate was collected with a 3′SL recovery of 74.9%. The resulting permeate was then subjected to process steps well known in the art, such as nanofiltration, treatment with activated charcoal and drying to form a white powder of 3′SL, with a protein content below 100 mg per kg dry solid, a DNA content below 10 ng per gram dry solid and an endotoxin content below 10000 EU per gram dry solid.

### Example 6. Purification of LNT from a Fermentation Broth Obtained with an Engineered E. coli Strain

Broth comprising LNT was produced via fermentation as described in Example 2. After fermentation, the resulting broth comprising LNT and antifoam was subjected to heat treatment above 60° C. Afterwards, the fermentation broth was subjected to filtration with a polypiperazine-amide membrane (TriSep UA60, Mann+Hummel) at pH 6.8 and at a temperature of 45° C. using an inlet pressure of 9.4 bar. During the first part of the filtration the retentate was concentrated from 72 L to 60 L. Afterwards, the retentate was subjected to diafiltration on said polypiperazine-amide membrane where RO-water is added to the retentate at a flow rate equal to the filtrate flow rate which averaged at 6.61 L filtrate/h/m² membrane surface. In this way, retentate volume is kept constant. This diafiltration was performed until a total filtrate volume of 1.3 times the retentate volume was obtained. Said filtration retained the biomass and the antifoam in the retentate and resulted into the formation of a permeate comprising said LNT. The permeate was collected with an LNT recovery of 84%. The resulting permeate was then subjected to process steps well known in the art, such as nanofiltration, treatment with activated charcoal and drying to form a white powder of LNT, with a protein content below 100 mg per kg dry solid, a DNA content below 10 ng per gram dry solid and an endotoxin content below 10000 EU per gram dry solid.

### Example 7. Flux Analysis During Biomass Removal from Fermentation Broth with Different Membranes

Broth comprising sialyllactose was produced via two fermentation processes as described in Example 2. After each fermentation, the biomass was removed from the fermentation broth via filtration and subsequent diafiltration as described below and the permeate was collected in fractions. The mass of these fractions and the time to collect them was measured. In this way, a flux could be determined by dividing the mass by the surface area and time.

In a first test, 33.9 kg of fermentation broth from the first fermentation was subjected to filtration with a polyethersulfone membrane (TriSep UF10, Mann+Hummel) at pH 7.0 and at a temperature of 45° C. using an inlet pressure of 5 bar. The retentate obtained was subjected to diafiltration at a constant volume until a total of 1.5 diafiltrations was obtained. During the filtration the flux decreased by 90% (see Table 2), suggesting the presence of a fouling effect of the broth on the membrane.

TABLE 2
Permeate data obtained during filtration of fermentation broth
with a polyethersulfone membrane (TriSep UF10, Mann + Hummel)
at pH 7.0 and at a temperature of 45° C. using an inlet pressure
of 5 bar and subsequent diafiltration of the retentate obtained

| Cumulative | | Permeate flux |
|---|---|---|
| mass (kg) | Diafiltrations | (L/h · m²) |
| 17.482 | 0.5 | 18.21 |
| 34.746 | 1.0 | 5.53 |
| 52.044 | 1.5 | 1.87 |

In a second test, 29.99 kg of fermentation broth from the second fermentation was subjected to filtration with a polypiperazine-amide membrane (TriSep UA60, Mann+Hummel) at pH 6.7 and at a temperature of 41° C. using an inlet pressure of 7 bar. The retentate obtained was subjected to diafiltration at a constant volume until a total of 5.2 diafiltrations was obtained. During the filtration the flux increased by 212% (see Table 3), without a fouling effect of the broth on the membrane observed.

TABLE 3
Permeate data during filtration of fermentation broth
with a polypiperazine-amide membrane (TriSep UA60,
Mann + Hummel) at pH 6.7 and at a temperature
of 41° C. using an inlet pressure of 7 bar and
subsequent diafiltration of the retentate obtained

| Cumulative | | Permeate flux |
|---|---|---|
| mass (kg) | Diafiltrations | (L/h · m²) |
| 23.58 | 0.8 | 22.7 |
| 46.89 | 1.6 | 38.9 |
| 69.32 | 2.3 | 33.0 |
| 91.77 | 3.1 | 35.1 |
| 114.05 | 3.8 | 39.8 |
| 134.89 | 4.5 | 43.4 |
| 154.98 | 5.2 | 50.2 |

### Example 8. Purification of 2′FL from a Fermentation Broth Obtained with an Engineered E. coli Strain

Broth comprising 2′FL was produced via fermentation as described in Example 2. After fermentation, fermentation broth comprising 2′FL and antifoam was subjected to heat treatment above 60° C. Afterwards, the fermentation broth was subjected to filtration with a polypiperazine-amide membrane (TriSep UA60, Mann+Hummel) at pH 6.5 and at a temperature of 45° C. using an inlet pressure of 8 bar. The retentate was first concentrated to 50% of the original broth volume. After reaching this retentate volume, water was added at a flow rate equal to the permeate flow rate. In this way, diafiltration is performed and the retentate volume remains at 50% of the original broth volume. Diafiltration was performed until a total of 3.9 diafiltrations was obtained. The average flux during the filtration was 14.71 L/h/m². Said filtration retained the biomass and the antifoam in the retentate and resulted into the formation of a permeate comprising said 2′FL. The permeate was collected with a 2′FL recovery of 91.6%. The resulting permeate was then subjected to process steps well known in the art, such as nanofiltration, treatment with activated charcoal and drying to form a white powder of 2′FL, with a protein content below 100 mg per kg dry solid, a DNA content below 10 ng per gram dry solid and an endotoxin content below 10000 EU per gram dry solid.

1.-21. (canceled)

22. A method of purifying a saccharide from a fermentation broth comprising the saccharide and biomass, wherein the fermentation broth originates from a fermentation of a cell producing the saccharide and wherein the biomass consists essentially of or consists of intact cells, disrupted cells, cell fragments, cell walls, phospholipids, cell membranes, proteins, protein fragments, polysaccharides, polynucleotides and large organic compounds produced by the cell of the fermentation, the method comprising:
filtration of the fermentation broth on a membrane and subsequent diafiltration on the membrane, wherein the membrane comprises:
a molecular weight cut-off ranging from 0.3 to 5 kDa, from 0.5 to 4 kDa, from 1 to 3.5 kDa, and/or from 1.5 to 3 kDa,
a pore size ranging from 0.001 to 0.01 μm, and/or
a monovalent ion rejection ranging from 1 to 50%, from 5 to 40%, and/or from 10 to 20%,
under conditions permissive to collect (1) a retentate comprising the biomass and (2) a permeate comprising essentially of all of the saccharide.

23. The method according to claim 22, wherein the membrane:
is selected from the group consisting of a composite membrane, a thin-film composite membrane, and a zwitterionic membrane,
is based on any one of polypiperazine-amide, polyamide, composite polyamide, composite fluoro polymer and zwitterions,
is any one of flat sheet membrane or a spiral-wound membrane,
is any one of an anionic, a cationic or a zwitterionic membrane,
has a rejection of NaCl ranging from 1 to 40%,
has a rejection of MgSO₄ ranging from 10 to 98%, and/or
has not been pre-treated with a liquid that contains any one or more of alcohol, organic sulfonic acid and sulfonate, and surfactant prior to its use in the filtration and diafiltration.

24. The method according to claim 22, wherein the filtration and/or diafiltration is/are performed:
at a temperature of the fermentation broth ranging from 4° C. to 55° C.,
using an inlet pressure ranging from 1 to 10 bar,
at a permeate flux of at least 5 L/m²/h, wherein the permeate flux is calculated as the volume in liter (L) of the permeate collected per hour (h) per square meter membrane area (m²) of the membrane,
at an initial permeate flux that is at least 10% of a pure water flux, wherein:
the initial permeate flux is calculated as the volume in liter (L) of the permeate collected per hour per square meter membrane area (m²) of the membrane of the filtration and/or diafiltration,
the pure water flux is calculated as the volume in liter (L) of pure water collected per hour (h) per square meter membrane area (m²) of the membrane of the filtration and/or diafiltration, and
the initial permeate flux and the pure water flux are measured in a filtration and/or diafiltration on the same membrane under identical conditions of temperature and pressure from the start of the filtration and/or diafiltration and for at least 10 minutes, for at least 20 minutes, for at least 30 minutes and/or for at least 1 hour.

25. The method according to claim 22, wherein the filtration and/or diafiltration is/are performed under conditions such that:
the decline in permeate flux in the filtration and/or diafiltration ranges from 0.1 to 15%,
the diafiltration is performed until a conductivity is reached for the retentate of ≤40 mS/cm,
the diafiltration is performed at a retentate volume of at least 50% of the original volume of the fermentation, and/or
at the end of the filtration and/or diafiltration the surface area of the membrane is covered for 0 to 10% with a fouling cake,
wherein the filtration is performed until a retentate volume is obtained of at least 50%, of the original volume of the fermentation,
wherein the diafiltration is performed until the original retentate volume has been collected at least once.

26. The method according to claim 22, wherein:
the temperature of the fermentation broth is adjusted to a temperature of from 0° C. to 130° C.,
the temperature of the fermentation broth is adjusted to a temperature of from 36° C. to 65° C., wherein the temperature is within 5° C. of a temperature at which the fermentation broth exhibits maximum turbidity, and/or
the fermentation broth is heat treated to a temperature ranging from 60° C. to 130° C. and/or from 80° C. to 122° C. prior to the filtration.

27. The method according to claim 22, wherein the pH of the fermentation broth:
has not been adjusted prior to the filtration, and/or
ranges from 2 to 7.

28. The method according to claim 22, wherein the biomass in the fermentation broth has a cell dry weight (CDW) that is at least 30 g/L.

29. The method according to claim 22, wherein the fermentation broth is not subjected to dilution prior to the filtration.

30. The method according to claim 22, wherein the saccharide is selected from the group consisting of monosaccharide, disaccharide, oligosaccharide, polysaccharide, neutral (non-charged) saccharide, negatively charged saccharide, sialylated saccharide, milk oligosaccharide, a mammalian milk oligosaccharide (MMO), a human milk oligosaccharide (HMO), lactose, sucrose, glucose, glycerol, galactose, fucose, mannose, N-acetylglucosamine, N-acetylgalactosamine, lactosamine, lacto-N-biose, 0-antigen, enterobacterial common antigen (ECA), the oligosaccharide repeats present in capsular polysaccharides, peptidoglycan, an amino-sugar, Lewis-type antigen oligosaccharide, an antigen of the human ABO blood group system, an animal oligosaccharide, an animal oligosaccharide selected from the grouplist consisting of N-glycans and O-glycans, a plant oligosaccharide, a plant oligosaccharide selected from the group consisting of N-glycans and O-glycans, fucosylated oligosaccharide, fucosylated oligosaccharide selected from the group consisting of 2′-fucosyllactose (2′FL), 3-fucosyllactose (3FL), 4-fucosyllactose (4FL), 6-fucosyllactose (6FL), 2′,3-difucosyllactose (diFL), lacto-N-fucopentaose I, lacto-N-neofucopentaose I, lacto-N-fucopentaose II, lacto-N-fucopentaose III, lacto-N-fucopentaose V, lacto-N-fucopentaose VI, lacto-N-neofucopentaose V, lacto-N-difucohexaose I, lacto-N-difucohexaose II, difucosyl-lacto-N-hexaose and difucosyl-lacto-N-neohexaose, sialylated oligosaccharide, sialylated oligosaccharide selected from the group consisting of 3′sialyllactose (3′SL), 6′sialyllactose (6′SL), sialyllacto-N-tetraose a (LSTa), sialyllacto-N-tetraose b (LSTb), sialyllacto-N-tetraose c (LSTc), sialyllacto-N-tetraose d (LSTd), disialyllacto-N-tetraose, disialyllacto-N-neotetraose, monosialyllacto-N-hexaose, disialyllacto-N-hexaose I, disialyllacto-N-hexaose II, monosialyllacto-N-neohexaose I, monosialyllacto-N-neohexaose II, disialyllacto-N-neohexaose, 3′-sialyl-3-fucosyllactose, fucodisialyllacto-N-hexaose, disialomonofucosyllacto-N-neohexaose, sialyllacto-N-fucohexaose II, disialyllacto-N-fucopentaose II and monofucosyldisialyllacto-N-tetraose, N-acetylglucosamine containing neutral (non-charged) oligosaccharide, N-acetylglucosamine containing neutral (non-charged) oligosaccharide selected from the group consisting of lacto-N-triose II (LN3), lacto-N-tetraose (LNT), lacto-N-neotetraose (LNnT), 6′-galactosyllactose, 3′-galactosyllactose, lacto-N-hexaose, lacto-N-neohexaose, para-lacto-N-hexaose, para-lacto-N-neohexaose, N-acetyllactosamine containing oligosaccharide, lacto-N-biose containing oligosaccharide, non-fucosylated neutral (non-charged) oligosaccharide, chitosan, chitosan comprising oligosaccharide, heparosan, chondroitin sulphate, glycosaminoglycan oligosaccharide, heparin, heparan sulphate, dermatan sulphate, hyaluronan, hyaluronic acid, and keratan sulphate.

31. The method according to claim 22, wherein the cell has been metabolically engineered to produce the saccharide.

32. The method according to claim 22, wherein the saccharide is accompanied in the fermentation broth by sialic acid, ashes, ashes comprising sulphates and phosphates, one or more monosaccharide(s), one or more activated monosaccharide(s), one or more phosphorylated monosaccharide(s) and/or one or more other saccharide(s).

33. The method according to claim 22, wherein the cell is a prokaryotic cell, yeast cell, bacterial cell, archaebacterial cell or fungal cell.

34. The method according to claim 22, wherein the cell is cultivated in:
culture medium comprising a carbon source comprising a monosaccharide, disaccharide, oligosaccharide, polysaccharide, polyol, glycerol, a complex medium including molasses, corn steep liquor, peptone, tryptone or yeast extract;
culture medium comprising a carbon source wherein the carbon source is selected from the group consisting of glucose, N-acetylglucosamine (GlcNAc), glycerol, fructose, sucrose, maltose, lactose, arabinose, malto-oligosaccharides, maltotriose, sorbitol, xylose, rhamnose, galactose, mannose, methanol, ethanol, trehalose, starch, cellulose, hemi-cellulose, molasses, corn-steep liquor, high-fructose syrup, acetate, citrate, lactate and pyruvate;
a chemically defined medium, and/or
a minimal salt medium comprising sulphate, phosphate, chloride, ammonium, calcium, magnesium, sodium, potassium, iron, copper, zinc, manganese, cobalt, and/or selenium.

35. The method according to claim 22, wherein the fermentation broth further comprises:
antifoam and/or proteins,
at least 0.01% antifoam (v/v), at least 0.1% antifoam (v/v) and/or at least 1% antifoam (v/v), and/or
antifoam and/or proteins wherein the antifoam and/or proteins is/are present in the retentate.

36. The method according to claim 22, wherein:
the purity of the saccharide in the fermentation broth is <70% on total dry solid before purification by the method,
the purity of the saccharide obtained in a purified saccharide solution at the end of the method is at least 70% on total dry solid, and/or
the yield of purification of the saccharide obtained in the purified saccharide solution at the end of the method is at least 60%.

37. The method according to claim 22, wherein the method further comprises any one or more of concentration, homogenization, clarification, clearing, centrifugation, decantation, dilution, pH adjustment, temperature adjustment, filtration, ultrafiltration, microfiltration, diafiltration, reverse osmosis, electrodialysis, electrodeionization, nanofiltration, dialysis, use of activated charcoal or carbon, use of solvents, use of alcohols, use of aqueous alcohol mixtures, use of charcoal, tangential flow high-performance filtration, tangential flow ultrafiltration, affinity chromatography, ion exchange, ion exchange chromatography, mixed bed ion exchange, hydrophobic interaction chromatography, gel filtration, ligand exchange chromatography, column chromatography, cation exchange adsorbent resin, anion exchange adsorbent resin, use of an adsorbent material, use of ion exchange resin, evaporation, vacuum evaporation, wiped film evaporation, falling film evaporation, pasteurization, enzymatic treatment, enzymatic treatment comprising incubation with one or more enzymes selected from the group consisting of glycosidase, lactase, β-galactosidase, fucosidase, sialidase, maltase, amylase, hexaminidase, glucuronidase, trehalase, and invertase, enzymatic treatment converting lactose, sucrose, malto-oligosaccharides, maltotriose, sorbitol, trehalose, starch, cellulose, hemi-cellulose, lignocellulose, molasses, corn-steep liquor and/or high-fructose syrup to monosaccharides, decolorization, drying, drying selected from the group consisting of spray drying, lyophilization, spray freeze drying, freeze spray drying, band drying, belt drying, vacuum band drying, vacuum belt drying, drum drying, roller drying, vacuum drum drying, vacuum roller drying, and agitated thin film drying, in any order.

38. The method according to claim 22, wherein a purified saccharide solution obtained at the end of the method:
has an ash content of ≤10%, ≤9%, ≤8%, ≤7%, ≤6% and/or ≤5% on total dry solid,
has an ash content of ≤10% on total dry solid with a lead content lower than 0.1 mg/kg dry solid,
has an ash content of ≤10% on total dry solid with an arsenic content lower than 0.2 mg/kg dry solid,
has an ash content of ≤10% on total dry solid with a cadmium content lower than 0.1 mg/kg dry solid,
has an ash content of ≤10% on total dry solid with a mercury content lower than 0.5 mg/kg dry solid,
has a protein content below 100 mg per kg dry solid, a DNA content below 10 ng per gram dry solid and/or an endotoxin content below 10000 EU per gram dry solid,
is free of DNA, recombinant DNA, proteins, proteins derived from a recombinant micro-organism and/or recombinant genetic material,
has less than 125 μg/kg of 3-MCPD, wherein the 3-MCPD is the sum of 3-monochloropropanediol (3-MCPD) and 3-MCPD fatty acid esters,
has less than 0.5 μg/kg epichlorohydrin,
is filter-sterilized, subjected to endotoxin removal and/or subjected to endotoxin removal by filtration through a 3 kDa filter,
has a conductivity of less than 10 mS/cm at a 300 g/L solution,
is further i) concentrated to a syrup of at least 20% dry matter, at least 30% dry matter and/or at least 40% dry matter; ii) crystallized; iii) dried to a powder or iv) granulated,
is further concentrated to a saccharide concentration of >100 g/L, and/or at a temperature of <80° C., between 20° C. to 50° C., and/or 30° C. to 45° C.,
is further concentrated by a method comprising using vacuum evaporation or reverse osmosis or nanofiltration,
is dried by any one or more of drying steps selected from the group consisting of spray drying, lyophilization, evaporation, precipitation, freeze drying, spray freeze drying, freeze spray drying, band drying, belt drying, vacuum band drying, vacuum belt drying, drum drying, roller drying, vacuum drum drying, vacuum roller drying, and agitated thin film drying,
comprises a saccharide that is concentrated to a concentration of >1.5 M and cooled to a temperature <25° C. and/or <8° C., to obtain crystalline material of the saccharide,
is spray-dried at a saccharide solution concentration of 20-60 (w/v), with a nozzle temperature of 110-150° C., and/or an outlet temperature of 60-80° C. and/or 65-70° C., and/or
is spray-dried at a saccharide solution concentration of 20-60 (w/v) with a nozzle temperature of 110-150° C., and/or an outlet temperature of 60-80° C. and/or 65-70° C., and wherein the pH of the purified saccharide solution is ranging from 2 to 7.

39. A purified saccharide solution, purified saccharide or purified saccharide mixture comprising a saccharide produced by the method according to claim 22.

40. A purified saccharide produced by the method according to claim 22, wherein a purified saccharide solution comprising the purified saccharide is i) dried, spray-dried or dried via an agitated thin film dryer; ii) lyophilized; iii) crystallized or iv) concentrated to a syrup of at least 20% dry matter, at least 30% dry matter and/or at least 40% dry matter.

41. A saccharide purified according to the method of claim 22, wherein the purified saccharide obtained after the method:
has an ash content of ≤10%, ≤9%, ≤8%, ≤7%, ≤6% and/or ≤5% on total dry solid,
has an ash content of ≤10% on total dry solid with a lead content lower than 0.1 mg/kg dry solid,
has an ash content of ≤10% on total dry solid with an arsenic content lower than 0.2 mg/kg dry solid,
has an ash content of ≤10% on total dry solid with a cadmium content lower than 0.1 mg/kg dry solid,
has an ash content of ≤10% on total dry solid with a mercury content lower than 0.5 mg/kg dry solid,
has a protein content below 100 mg per kg dry solid, a DNA content below 10 ng per gram dry solid and/or an endotoxin content below 10000 EU per gram dry solid,
is free of DNA, recombinant DNA, proteins, proteins derived from a recombinant micro-organism, and/or recombinant genetic material,
has less than 125 μg/kg of 3-MCPD, wherein the 3-MCPD is the sum of 3-monochloropropanediol (3-MCPD) and 3-MCPD fatty acid esters,
has less than 0.5 μg/kg epichlorohydrin, and/or
has a conductivity of less than 10 mS/cm at a 300 g/L solution.

42. Dried powder of purified saccharide solution produced by the method according to claim 38, wherein the dried powder:
contains ≤15%-wt. of water,
has a mean particle size of 50 to 250 μm, as may be determined by laser diffraction,
has an ash content of ≤10% on total dry solid,
has an ash content of ≤10% on total dry solid with a lead content lower than 0.1 mg/kg dry solid,
has an ash content of ≤10% on total dry solid with an arsenic content lower than 0.2 mg/kg dry solid,
has an ash content of ≤10% on total dry solid with a cadmium content lower than 0.1 mg/kg dry solid,
has an ash content of ≤10% on total dry solid with a mercury content lower than 0.5 mg/kg dry solid,
has a protein content below 100 mg per kg dry solid, a DNA content below 10 ng per gram dry solid and/or an endotoxin content below 10000 EU per gram dry solid,
is free of DNA, recombinant DNA, proteins, proteins derived from the recombinant micro-organism and/or recombinant genetic material,
has less than 125 μg/kg of 3-MCPD, wherein the 3-MCPD is the sum of 3-monochloropropanediol (3-MCPD) and 3-MCPD fatty acid esters,
has less than 0.5 μg/kg epichlorohydrin, and/or
is a spray-dried powder.
